(19) United States
(12) Patent Application Publication
(10) Pub. No.: US 2021/0063375 A1
(43) Pub. Date: Mar. 4, 2021

(54) SCREENING AND CONFIRMATION METHOD FOR VETERINARY DRUGS AND ADDITIVES IN ANIMAL-DERIVED FOOD

(71) Applicant: Henan Institute of Veterinary Drug and Feed Control, Zhengzhou (CN)

(72) Inventors: Zhiming WU, Zhengzhou (CN); Xiaoou SU, Zhengzhou (CN); Lei MENG, Zhengzhou (CN); Ningpeng WU, Zhengzhou (CN); Li PENG, Zhengzhou (CN); Lingping LI, Zhengzhou (CN); Panpan ZHANG, Zhengzhou (CN); Yanling GAO, Zhengzhou (CN); Huisu LI, Zhengzhou (CN); Jie YANG, Zhengzhou (CN); Cong YUAN, Zhengzhou (CN); Peilong WANG, Zhengzhou (CN)

(21) Appl. No.: 17/007,239

(22) Filed: Aug. 31, 2020

(30) Foreign Application Priority Data
Aug. 31, 2019 (CN) 201910822246.X
Sep. 20, 2019 (CN) 201910891387.7

(51) Int. Cl. G01N 33/15; G01N 30/88

(52) U.S. Cl. CPC G01N 33/15; G01N 2030/8813; G01N 30/88

(57) ABSTRACT

A screening and confirmation method for veterinary drugs and additives in animal-derived food, including: preparation of standard working solution, pretreatment of samples to be tested, establishment of database, sample detection, compound screening and compound confirmation. The disclosure employs ultra high performance liquid chromatography-quadrupole/electrostatic field orbitrap high resolution mass spectrometry, and enables the simultaneous detection of 207 veterinary drugs and additives in animal-derived food within 22 min.

### CROSS-REFERENCE TO RELATED APPLICATIONS

This application claims the benefit of priority from Chinese Patent Application No. 201910891387.7, filed on Sep. 20, 2019. The content of the aforementioned application, including any intervening amendments thereto, is incorporated herein by reference in its entirety.

### TECHNICAL FIELD

This application relates to detection of animal-derived food, and more particularly to a screening and confirmation method for veterinary drugs and additives in animal-derived food.

### BACKGROUND

Veterinary drugs refer to substances used to prevent, treat, diagnose animal diseases or purposely regulate animal physiological functions, which generally include antibiotics, anthelmintics, growth promoters, antiprotozoals, trypanosome-killing substances, sedatives and β-adrenoceptor blockers.

Recently, the animal husbandry has achieved rapid development in China, which is attributed to the development and rational use of veterinary drugs. Veterinary drugs have played an important role in reducing the morbidity and mortality of livestock, promoting animal growth, improving the quality of animal-derived products and improving feed utilization. However, at present, the veterinary drugs are often abused with the dosage far exceeding the therapeutic dose for animal diseases, resulting in drug residues in the animal-derived food. Through the long-term accumulation, most of the veterinary drugs show significant toxicity. For example, residual lincosamines can cause renal dysfunction and increased drug resistance of Gram-positive bacteria; residual macrolide drugs can cause allergic reactions and also lead to the spread of strains carrying drug-resistant factors; and chloramphenicol can cause anemia, gray baby syndrome and leukemia, etc. Therefore, the European Union and China have established markers for related drug residues and maximum residue limits in different animal-derived foods, and also stipulated the types of veterinary drugs banned from animal husbandry, but the abuse of veterinary drugs still cannot be precluded.

Public notice No. 235 of the Ministry of Agriculture of China has clearly stipulated the maximum residue limit of each drug in animal tissues. However, the irrational and illegal use of veterinary drugs still can be found in the veterinary drug inspection or in the farms. In addition to veterinary drugs, the pesticide residues in animal foods also raised widespread concern in 2017 due to the “fipronil” poison egg incident. Since the residues of pesticides and veterinary drugs in animal-derived foods directly or indirectly endanger the human health, and destroy the ecological environment, it is necessary to comprehensively regulate and monitor the use of veterinary drugs and additives in animal-derived foods.

The development of the detection technology enables the simultaneous detection of multiple categories of pesticides and veterinary drugs, and also diversifies the pre-processing technologies and instrument analysis tools. Developed countries such as Europe and the United States have made a great progress in the detection of residual pesticides and veterinary drugs. Specifically, the Food and Drug Administration (FDA) have established a method for detecting more than 150 kinds of veterinary drugs in milk using LC-TOF-MS, which has a detection limit of 10 ng/mL or lower for 50% of the veterinary drugs. The U.S. Department of Agriculture have established a detection method for 120 kinds of veterinary drugs in 11 categories in bovine kidney using HPLC-Quadrupole Mass Spectrometry, which has a detection limit of 1-100 ng/g and a recovery rate of 29%-192%.

At present, the multi-residue detection in China is mainly performed by HPLC-Quadrupole Mass Spectrometry and liquid chromatography-high resolution mass spectrometry (LC-HRMS). Among them, the traditional HPLC-Quadrupole Mass Spectrometry has the characteristics of high sensitivity and accurate quantification. However, it also has the shortcomings of low resolution, high occurrence rate of false positives, limited detectable drugs and large complexity in the establishment of methods.

### SUMMARY

An object of this application is to provide a screening and confirmation method for 207 veterinary drugs and additives in animal-derived food to solve the above problems.

The technical solutions of the application are specifically described as follows.

This application provides a screening and confirmation method for veterinary drugs and additives in animal-derived food, comprising:

(1) dissolving 10 mg of each reference standard of the veterinary drugs and additives followed by diluting to 10 mL in a volumetric flask to prepare a 1 mg/mL stock solution of each reference standard;

wherein the veterinary drugs and additives are selected from:

(a) sulfonamides: sulfadimidine, sulformethoxine, sulfisoxazole, sulfaguanidine, sulfamerazine, sulfamethylthiadizaole, sulfamethoxazole, sulfamethoxydiazine, sulfamethoxypyridazine, sulfamonomethoxine, sulfamethazole, sulfapyridine, sulfaquinoxaline, sulfathiazole, sulfisomidine, trimethoprim, diaveridine, phthalylsulfathiazole, sulfabenzamide, sulfacetamide, sulfachlorpyridazine, sulfachloropyrazine, sulfadiazine and sulfadimethoxine;

(b) fluoroquinolones: lomefloxacin, marbofloxacin, nadifloxacin, nalidixic acid, norfloxacin, ofloxacin, orbifloxacin, oxolinic acid, pipemidic acid, pefloxacin, cinoxacin, sarafloxacin, sparfloxacin, ciprofloxacin, danofloxacin, difloxacin, enoxacin, enrofloxacin, fleroxacin, flumequine and gatifloxacin;

(c) benzimidazoles: mebendazole, mebendazole-amine, 2-aminofluorobenzimidazole, oxfendazole, oxibendazole, parbendazole, 5-hydroxythiabendazole, thiabendazole, albendazole, albendazole sulfone, albendazole sulfoxide, albendazole aminosulfone, benzimidazole, febantel and lobendazole;

(d) stimulants: cyproheptadine, fenoterol, labetalol, penbutolol, phenylethanolamine A, ractopamine, salbutamol, salmeterol, terbutaline, mabuterol, tulobuterol, bambuterol, brombuterol, cimaterol, cimbuterol, clenbuterol, clenproperol and clonidine;

(e) hormones: chlormadinone acetate, 17a-methyltestosterone, clobetasol propionate, clobetasone butyrate, corticosterone, hydrocortisone, cortisone acetate, cortisone, deflazacort, dexamethasone, diflorasone diacetate, epitestosterone, estradiol benzoate, fludrocortisone acetate, flumetasone pivalate, flumethasone, fluocinolone acetonide, fluorometholone, flurandrenolide, fluticasone propionate, nandrolone propionate, halcinonide, dihydrodiethylstilbestrol, medroxyprogesterone acetate, megestrol acetate, melengestrol acetate, methandienone, methylprednisolone, norethindrone, nandrolone propionate, levonorgestrel, prednicarbate, prednisolone, prednisone, progesterone, dehydrotestosterone, testosterone, trenbolone, triamcinolone acetonide, beclomethasone, betamethasone dipropionate, betamethasone valerate and betamethasone;

(f) nitroimidazoles: dimetridazole, ipronidazole, metronidazole, ornidazole, hydroxydimetridazole, ronidazole, secnidazole and tinidazole;

(g) antiviral substances: amantadine and rimantadine;

(h) lincosamides: clindamycin and lincomycin;

(i) amphenicols: chloramphenicol, florfenicol and thiamphenicol;

(j) penicillins: ceftiofur, flucloxacillin, cloxacillin, nafcillin, piperacillin and penicillin G;

(k) quinoxalines: quinocetone;

(l) antipyretics and analgesics: phenacetin, salicylic acid, amidopyrine, antipyrine, dapsone, flunixin meglumine and acetaminophen;

(m) psychoactive drugs: haloperidol, imipramine, nitrazepam, oxazepam, pemoline, perphenazine, promethazine, propionylpromazine hydrochloride, sulpiride, xylazine, acepromazine, anisopirol, azaperone, carbamazepine, chloropromazine, diazepam, diphenhydramine, droperidol, estazolam, clarithromycin, tiamulin, erythromycin, kitasamycin, tilmicosin and tylosin;

(n) anthelmintics: toltrazuril sulfoxide, clopidol, dinitolmide, halofuginone and levamisole;

(o) pesticides: buprofezin, carboxin, 3-hydroxycarbofuran, clothianidin, cyromazine, diuron, ethopabate, fipronil, fluridone, acephate, hexazinone, imazalil, imidacloprid, linuron, metribuzin, myclobutanil, acetamiprid, norflurazon, propyzamide, atrazine, simazine, desethyl atrazine, azoxystrobin and benoxacor;

(p) other substances: scopolamine, valnemulin and atropine;

the norfloxacin is dissolved with water and diluted with methanol to 10 mL; each of the pesticides is dissolved with acetone and then diluted with methanol to 10 mL; each of the diaveridine, albendazole, albendazole sulfone and febantel is dissolved with formic acid and diluted with methanol to 10 mL; and the rest substances are dissolved with a mixed solvent of methanol and ammonia water or a mixed solvent of methanol and dimethyl sulfoxide, and then diluted with methanol to 10 mL;

(2) pipetting 100 μL of the stock solution of each reference standard in the same category in Table 1 to a volumetric flask followed by diluting with methanol to 10 mL to accordingly prepare 16 first mixed standard solutions, wherein a concentration of each reference standard in the corresponding first mixed standard solution thereof is g/mL; pipetting 100 μL of each first mixed standard solution to another volumetric flask followed by diluting with methanol to 10 mL to prepare a second mixed standard solution, wherein each reference standard in the second mixed standard solution has a concentration of 100 ng/mL; and pipetting 100 μL, 500 μL, 1 mL and 5 mL, respectively, to a volumetric flask followed by diluting with a 20% aqueous methanol solution to 10 mL to prepare a series of mixed standard working solutions, wherein each reference standard in the series of mixed standard working solutions has a concentration of 1 ng/mL, 5 ng/mL, 10 ng/mL and 50 ng/mL, respectively;

(3) adding 5 g of a sample to a 50 mL centrifuge tube; adding an extracting solution followed by vortex mixing; shaking and centrifuging the centrifuge tube; transferring a first supernatant to another 50 mL centrifuge tube;

if the sample is muscle or poultry egg, treating a precipitate with the extracting solution again to collect an extract; combining the first supernatant with the extract to produce a mixture; adding 6 g of anhydrous sodium sulfate and 1.5 g of sodium chloride to the mixture followed by vortex mixing for 30 s and standing; and centrifuging the another 50 mL centrifuge tube to produce a second supernatant;

if the sample is cow's milk or goat's milk, adding 4 g of anhydrous sodium sulfate and 1 g of sodium chloride to the first supernatant followed by vortex mixing for 30 s and standing; and centrifuging the another 50 mL centrifuge tube to produce a second supernatant;

(4) activating and equilibrating a solid phase extraction column sequentially with 4 mL of methanol and 4 mL of water; rinsing the solid phase extraction column with 2 mL of the second supernatant and discarding a first effluent; loading 5 mL of the second supernatant to the solid phase extraction column and collecting a second effluent with a centrifuge tube; drying 4 mL of the second effluent in nitrogen flow at 40° C.; adding 200 μL of methanol to the dried product followed by vortex mixing for 10 s; adding 800 μL of pure water followed by vortex mixing for 30 s to produce a sample solution; and filtering the sample solution with 0.22 μm nylon filter membrane and collecting a filtrate for use;

(5) diluting each of the first mixed standard solutions such that each reference standard in the corresponding diluted first mixed standard solution thereof has a concentration of 1 μg/mL; subjecting each of the diluted first mixed standard solutions to full scan and secondary scan in positive and negative modes using a ultra high performance liquid chromatography-quadrupole/electrostatic field orbitrap high resolution mass spectrometer; analyzing the scan results to obtain information of each reference standard using Trace Finder software of Q-Exactive plus; and establishing a mass spectrometry database based on the information of each reference standard; wherein the information of each reference standard comprises retention time, precise molecular weight of parent ion, addition mode, fragment ions, isotope abundance ratio and a fragment ion mass spectrometry generated through the superposition of fragment ion mass spectrometries obtained at collision energies of 20 eV, 40 eV and 60 eV; the isomerides are further identified through the analysis of single standard reference; and the mass spectrometry database is shown in Table 1;

TABLE 1
Mass spectrometry library information

| | | Molecular | Addition | Parent | | | Retention |
|---|---|---|---|---|---|---|---|
| Category | Compound | formula | mode | ion | Fragment 1 | Fragment 2 | time (min) |
| | Sulfamethazine | C₁₂H₁₄N₄O₂S | M + H | 279.09102 | 156.01138 | 124.08694 | 8.44 |
| | Sulformethoxine | C₁₂H₁₄N₄O₄S | M + H | 311.08085 | 156.01138 | 140.04545 | 9.55 |
| | Sulfisoxazole | C₁₁H₁₃N₃O₃S | M + H | 268.07504 | 156.0111 | 108.04462 | 9.83 |
| | Sulfaguanidine | C₇H₁₀N₄O₂S | M + H | 215.05972 | 156.01138 | 108.04439 | 3.43 |
| | Sulfamerazine | C₁₁H₁₂N₄O₂S | M + H | 265.07537 | 190.02809 | 156.01138 | 7.89 |
| | Sulfamethylthiadizaole | C₉H₁₀N₄O₂S₂ | M + H | 271.03179 | 156.01138 | 108.04439 | 8.31 |
| | Sulfamethoxazole | C₁₀H₁₁N₃O₃S | M + H | 254.05939 | 156.01138 | 108.04439 | 9.57 |
| | Sulfamethoxydiazine | C₁₁H₁₂N₄O₃S | M + H | 281.07029 | 108.04465 | 156.01115 | 8.53 |
| | Sulfamethoxypyridazine | C₁₁H₁₂N₄O₃S | M + H | 281.07029 | 215.09274 | 156.01138 | 8.41 |
| | Sulfamonomethoxine | C₁₁H₁₂N₄O₃S | M + H | 281.07029 | 215.09274 | 156.01138 | 8.9 |
| Sulfonamides | Sulfamethazole | C₁₁H₁₃N₃O₃S | M + H | 268.07504 | 156.0111 | 108.04462 | 8.03 |
| | Sulfapyridinee | C₁₁H₁₁N₃O₂S | M + H | 250.06447 | 184.08692 | 156.01138 | 7.55 |
| | Sulfaquinoxaline | C₁₄H₁₂N₄O₂S | M + H | 301.07537 | 156.01138 | 108.04439 | 10.39 |
| | Sulfathiazole | C₉H₉N₃O₂S₂ | M + H | 256.02089 | 156.01138 | 108.04439 | 7.28 |
| | Sulfisomidine | C₁₂H₁₄N₄O₂S | M + H | 279.09102 | 124.08692 | 108.04439 | 6.57 |
| | Trimethoprim | C₁₄H₁₈N₄O₃ | M + H | 291.14517 | 275.11387 | 261.09822 | 7.28 |
| | Diaveridine | C₁₃H₁₆N₄O₂ | M + H | 261.1346 | 245.1033 | 123.06652 | 7 |
| | Phthalylsulfathiazole | C₁₇H₁₃N₃O₅S₂ | M + H | 404.03694 | 149.02313 | 256.02032 | 9.07 |
| | Sulfabenzamide | C₁₃H₁₂N₂O₃S | M + H | 277.06414 | 156.01138 | 108.04439 | 10.32 |
| | Sulfacetamide | C₈H₁₀N₂O₃S | M + H | 215.04849 | 173.03792 | 156.01138 | 6.73 |
| | Sulfachlorpyridazine | C₁₀H₉ClN₄O₂S | M + H | 285.02075 | 156.01138 | 108.04439 | 9.25 |
| | Sulfachloropyrazine | C₁₀H₉ClN₄O₂S | M + H | 285.02075 | 130.0166 | 156.01111 | 10.35 |
| | Sulfadiazine | C₁₀H₁₀N₄O₂S | M + H | 251.05972 | 156.01138 | 108.04439 | 7.13 |
| | Sulfadimethoxine | C₁₂H₁₄N₄O₄S | M + H | 311.08085 | 156.07675 | 108.04439 | 10.43 |
| Fluoroquinolones | Lomefloxacin | C₁₇H₁₉F₂N₃O₃ | M + H | 352.14672 | 308.1569 | 265.1147 | 7.58 |
| | Marbofloxacin | C₁₇H₁₉FN₄O₄ | M + H | 363.14631 | 320.10411 | 72.08078 | 7.17 |
| | Nadifloxacin | C₁₉H₂₁FN₂O₄ | M + H | 361.15581 | 343.14389 | 283.08725 | 10.76 |
| | Nalidixic acid | C₁₂H₁₂N₂O₃ | M + H | 233.09207 | 205.06077 | 187.0502 | 11.43 |
| | Norfloxacin | C₁₆H₁₈FN₃O₃ | M + H | 320.1405 | 276.15067 | 233.10847 | 7.24 |
| | Ofloxacin | C₁₈H₂₀FN₃O₄ | M + H | 362.15106 | 318.16123 | 261.10338 | 7.31 |
| | Orbifloxacin | C₁₉H₂₀F₃N₃O₃ | M + H | 396.15295 | 352.16312 | 295.10527 | 7.89 |
| | Oxolinic acid | C₁₃H₁₁NO₅ | M + H | 262.071 | 244.06043 | 234.0397 | 9.95 |
| | Pipemidic acid | C₁₄H₁₇N₅O₃ | M + H | 304.14042 | 290.16632 | 233.10847 | 6.75 |
| | Pefloxacin | C₁₇H₂₀FN₃O₃ | M + H | 334.15615 | 217.10839 | 189.0779 | 7.35 |
| | Cinoxacin | C₁₂H₁₀N₂O₅ | M + H | 263.06625 | 235.07133 | 217.06077 | 9.47 |
| | Sarafloxacin | C₂₀H₁₇F₂N₃O₃ | M + H | 386.13107 | 342.14125 | 299.09905 | 8.16 |
| | Sparfloxacin | C₁₉H₂₂F₂N₄O₃ | M + H | 393.17327 | 349.18344 | 292.12559 | 8.22 |
| | Ciprofloxacin | C₁₇H₁₈FN₃O₃ | M + H | 332.1405 | 288.15067 | 245.10847 | 7.37 |
| | Danofloxacin | C₁₉H₂₀FN₃O₃ | M + H | 358.15615 | 314.16632 | 283.12412 | 7.54 |
| | Difloxacin | C₂₁H₁₉F₂N₃O₃ | M + H | 400.14672 | 356.15689 | 299.09905 | 8.27 |
| | Enoxacin | C₁₅H₁₇FN₄O₃ | M + H | 321.13575 | 277.14592 | 257.13969 | 7.13 |
| | Enrofloxacin | C₁₉H₂₂FN₃O₃ | M + H | 360.1718 | 316.18197 | 245.10847 | 7.73 |
| | Fleroxacin | C₁₇H₁₈F₃N₃O | M + H | 370.1373 | 326.14747 | 269.08962 | 7.36 |
| | Flumequine | C₁₄H₁₂FNO₃ | M + H | 262.0874 | 244.07683 | 220.04045 | 11.59 |
| | Gatifloxacin | C₁₉H₂₂FN₃O₄ | M + H | 376.16671 | 332.17688 | 289.13468 | 7.97 |
| Stimulants | Cyproheptadine | C₂₁H₂₁N | M + H | 288.17468 | 191.08553 | 96.08078 | 10.67 |
| | Fenoterol | C₁₇H₂₁NO₄ | M + H | 304.15433 | 286.14377 | 135.08044 | 6.57 |
| | Labetalol | C₁₉H₂₄N₂O₃ | M + H | 329.18597 | 311.1754 | 294.14886 | 8.97 |
| | Penbutolol | C₁₈H₂₉NO₂ | M + H | 292.22711 | 236.16451 | 201.12739 | 11.08 |
| | Phenylethanolamine A | C₁₉H₂₄N₂O₄ | M + H | 345.18088 | 327.17032 | 150.09134 | 10.03 |
| | Ractopamine | C₁₈H₂₃NO₃ | M + H | 302.17507 | 284.16451 | 164.10699 | 7.56 |
| | Salbutamol | C₁₃H₂₁NO₃ | M + H | 240.15942 | 148.07569 | 166.08625 | 5.75 |
| | Salmeterol | C₂₅H₃₇NO₄ | M + H | 416.27954 | 398.26897 | 380.2584 | 11.03 |
| | Terbutaline | C₁₂H₁₉NO₃ | M + H | 226.14377 | 170.08117 | 152.07061 | 5.71 |
| | Mabuterol | C₁₃H₁₈ClF₃N₂O | M + H | 311.11325 | 237.03951 | 217.03343 | 9 |
| | Tulobuterol | C₁₂H₁₈ClNO | M + H | 228.11497 | 172.05237 | 154.0418 | 8.25 |
| | Bambuterol | C₁₈H₂₉N₃O₅ | M + H | 368.218 | 312.1554 | 294.14483 | 8.65 |
| | Brombuterol | C₁₂H₁₈Br₂N₂O | M + H | 364.98586 | 290.9127 | 211.99436 | 8.71 |
| | Cimaterol | C₁₂H₁₇N₃O | M + H | 220.14444 | 202.13387 | 160.08692 | 5.93 |
| | Cimbuterol | C₁₃H₁₉N₃O | M + H | 234.16009 | 160.08656 | 143.06007 | 6.61 |
| | Clenbuterol | C₁₂H₁₈Cl₂N₂O | M + H | 277.0869 | 259.07633 | 203.01373 | 8.31 |
| | Clenproperol | C₁₁H₁₆Cl₂N₂O | M + H | 263.07125 | 245.06068 | 203.01373 | 7.85 |
| | Clonidine | C₉H₉Cl₂N₃ | M + H | 230.02463 | 212.99808 | / | 6.77 |
| Hormones | Chlormadinone acetate | C₂₃H₂₉ClO₄ | M + H | 405.18271 | 309.1849 | 267.1743 | 15.67 |
| | 17a-methyltestosterone | C₂₀H₃₀O₂ | M + H | 303.23186 | 285.22129 | 267.21073 | 13.63 |
| | Clobetasol propionate | C₂₅H₃₂ClFO₅ | M + H | 467.19951 | 263.143 | 147.0804 | 15.08 |
| | Clobetasone butyrate | C₂₆H₃₂ClFO₅ | M + H | 479.19951 | 279.138 | 343.1459 | 16.13 |
| | Corticosterone | C₂₁H₃₀O₄ | M + H | 347.22169 | 329.21112 | 311.20056 | 11.61 |
| | Hydrocortisone | C₂₁H₃₀O₅ | M + H | 363.2166 | 327.19547 | 309.18491 | 10.34 |
| | Cortisone acetate | C₂₃H₃₀O₆ | M + H | 403.21152 | 343.19038 | 163.11174 | 12.53 |
| | Cortisone | C₂₁H₂₈O₅ | M + H | 361.20095 | 163.1117 | 121.0648 | 10.45 |
| | Deflazacort | C₂₅H₃₁NO₆ | M + H | 442.22241 | 142.0499 | 312.1958 | 12.39 |
| | Dexamethasone | C₂₂H₂₉FO₅ | M + H | 393.20718 | 355.19039 | 337.17982 | 11.21 |
| | Diflorasone diacetate | C₂₆H₃₂F₂O₇ | M + H | 495.21889 | 121.0648 | 317.1536 | 13.91 |
| | Epitestosterone | C₁₉H₂₈O₂ | M + H | 289.21621 | 97.0648 | 109.0648 | 13.93 |
| | Estradiol benzoate | C₂₅H₂₈O₃ | M + H | 377.21112 | 279.1743 | 321.1849 | 12.22 |
| | Fludrocortisone acetate | C₂₃H₃₁FO₆ | M + H | 423.21774 | 181.1023 | 239.143 | 12.22 |
| | Flumetasone pivalate | C₂₇H₃₆F₂O₆ | M + H | 495.25527 | 57.0699 | 121.0648 | 15.25 |
| | Flumethasone | C₂₂H₂₈F₂O₅ | M + H | 411.19776 | 235.1117 | 277.1587 | 11.2 |
| | Fluocinolone acetonide | C₂₄H₃₀F₂O₆ | M + H | 453.20832 | 121.0648 | 337.1434 | 11.86 |
| | Fluorometholone | C₂₂H₂₉FO₄ | M + H | 377.21226 | 279.1743 | 135.0804 | 12.22 |
| | Flurandrenolide | C₂₄H₃₃FO₆ | M + H | 437.23339 | 361.1798 | 105.0699 | 12.01 |
| | Fluticasone propionate | C₂₅H₃₁F₃O₅S | M + H | 501.19171 | 121.0648 | 275.143 | 15.08 |
| | Nandrolone propionate | C₂₁H₃₀O₃ | M + H | 331.22677 | 257.18999 | 239.17943 | 17.38 |
| | Halcinonide | C₂₄H₃₂ClFO₅ | M + H | 455.19951 | 121.0648 | 377.1514 | 14.98 |
| | Dihydrodiethylstilbestrol | C₁₈H₂₂O₂ | M + H | 271.16926 | 109.1117 | 253.1587 | 11.92 |
| | Medroxyprogesterone | C₂₄H₃₄O₄ | M + H | 387.25299 | 123.0804 | 327.2319 | 15.81 |
| | acetate | | | | | | |
| | Megestrol acetate | C₂₄H₃₂O₄ | M + H | 385.23734 | 267.1743 | 224.156 | 15.53 |
| | Melengestrol acetate | C₂₅H₃₂O₄ | M + H | 397.23734 | 279.1743 | 337.2162 | 15.82 |
| | Methandienone | C₂₀H₂₈O₂ | M + H | 301.21621 | 283.20564 | 149.13248 | 12.73 |
| | Methylprednisolone | C₂₂H₃₀O₅ | M + H | 375.2166 | 161.0961 | 135.0804 | 10.99 |
| | Norethindrone | C₂₀H₂₆O₂ | M + H | 299.20056 | 109.0648 | 281.19 | 13.12 |
| | Nandrolone propionate | C₁₈H₂₆O₂ | M + H | 275.20056 | 109.06499 | 239.17885 | 12.47 |
| | Levonorgestrel | C₂₁H₂₈O₂ | M + H | 313.21621 | 109.0648 | 245.19 | 14.23 |
| | Prednicarbate | C₂₇H₃₆O₈ | M + H | 489.24829 | 147.0804 | 289.1587 | 14.91 |
| | Prednisolone | C₂₁H₂₈O₅ | M + H | 361.20095 | 147.0804 | 171.0804 | 10.2 |
| | Prednisone | C₂₁H₂₆O₅ | M + H | 359.1853 | 341.17474 | 237.12739 | 10.27 |
| | Progesterone | C₂₁H₃₀O₂ | M + H | 315.23186 | 109.06479 | 97.06479 | 15.76 |
| | Dehydrotestosterone | C₁₉H₂₆O₂ | M + H | 287.20056 | 121.0647 | 135.1167 | 12.22 |
| | Testosterone | C₁₉H₂₈O₂ | M + H | 289.21621 | 109.06479 | 97.06479 | 13.03 |
| | Trenbolone | C₁₈H₂₂O₂ | M + H | 271.16926 | 253.15869 | 199.11174 | 11.92 |
| | Triamcinolone acetonide | C₂₄H₃₁FO₆ | M + H | 435.21774 | 397.201 | 339.1591 | 11.69 |
| | Beclomethasone | C₂₂H₂₉ClO₅ | M + H | 409.17763 | 391.1671 | 279.1743 | 11.45 |
| | Betamethasone dipropionate | C₂₈H₃₇FO₇ | M + H | 505.25961 | 279.1741 | 319.1693 | 15.45 |
| | Betamethasone valerate | C₂₇H₃₇FO₆ | M + H | 477.26469 | 279.1743 | 147.0804 | 14.55 |
| | Betamethasone | C₂₂H₂₉FO₅ | M + H | 393.20718 | 355.19039 | 279.17434 | 11.14 |
| Nitroimidazoles | Dimetridazole | C₅H₇N₃O₂ | M + H | 142.0611 | 112.06311 | 95.06037 | 7.17 |
| | Ipronidazole | C₇H₁₁N₃O₂ | M + H | 170.0924 | 140.09441 | 123.09167 | 10.33 |
| | Metronidazole | C₆H₉N₃O₃ | M + H | 172.07167 | 128.04545 | 82.05255 | 6.39 |
| | Omidazole | C₇H₁₀ClN₃O₃ | M + H | 220.04835 | 128.04547 | 203.14304 | 8.77 |
| | Hydroxydimetridazole | C₅H₇N₃O₃ | M + H | 158.05602 | 140.0455 | 110.0475 | 6.42 |
| | Ronidazole | C₆H₈N₄O₄ | M + H | 201.06183 | 140.04545 | 110.04746 | 7.03 |
| | Secnidazole | C₇H₁₁N₃O₃ | M + H | 186.08732 | 128.0455 | 111.0427 | 7.56 |
| | Tinidazole | C₈H₁₃N₃O₄S | M + H | 248.06995 | 154.0611 | 110.08384 | 8.12 |
| Antiviral | Amantadine | C₁₀H₁₇N | M + H | 152.14338 | 135.11683 | 107.08553 | 7.48 |
| drugs | Rimantadine; | C₁₂H₂₁N | M + H | 180.17468 | 163.14778 | 81.0704 | 9 |
| Lincosamides | Clindamycin | C₁₈H₃₃ClN2O₅S | M + H | 425.18715 | 126.12773 | / | 8.69 |
| | Lincomycin | C₁₈H₃₄N₂O₆S | M + H | 407.221 | 126.12773 | 359.21766 | 6.76 |
| Amphenicols | Chloramphenicol | C₁₁H₁₂Cl₂N₂O₅ | M − H | 321.00505 | 257.03346 | 194.04588 | 10.11 |
| | Florfenicol | C₁₂H₁₄Cl₂FNO₄S | M − H | 355.99319 | 335.98696 | 185.02779 | 9.96 |
| | Thiamphenicol | C₁₂H₁₅Cl₂NO₅S | M − H | 353.99752 | 290.02593 | 185.02779 | 8.27 |
| β-Lactams | Ceftiofur | C₁₉H₁₇N₅O₇S₃ | M + H | 524.03629 | 241.03899 | 210.02065 | 9.63 |
| | Flucioxacillin | C₁₉H₁₇ClFN₃O₅S | M + H | 454.06342 | 160.04242 | 114.03742 | 12.5 |
| | Cioxacillin | C₁₉H₁₈ClN₃O₅S | M + H | 436.07285 | 277.0374 | 206.0367 | 12.26 |
| | Nafcillin | C₂₁H₂₂N₂O₅S | M + H | 415.13222 | 199.0754 | 171.0441 | 12.54 |
| | Piperacillin | C₂₃H₂₇N₅O₇S | M + H | 518.1704 | 143.0815 | 115.0502 | 10.16 |
| | Penicillin G | C₁₆H₁₈N₂O₄S | M + H | 335.106 | 217.06414 | 176.07061 | 10.77 |
| Quinoxalines | Quinocetone | C₁₈H₁₄N₂O₃ | M + H | 307.10772 | 143.06018 | 273.10178 | 11.99 |
| Antipyretics | Phenacetin | C₁₀H₁₃NO₂ | M + H | 180.10191 | 110.0602 | 138.09109 | 10.11 |
| and | Salicylic acid | C₇H₆O₃ | M − H | 137.02442 | 93.0346 | 65.0397 | 3.74 |
| analgesics | Amidopyrine | C₁₃H₁₇N₃O | M + H | 232.14444 | 113.10738 | 98.08418 | 6.46 |
| | Antipyrine | C₁₁H₁₂N₂O | M + H | 189.10224 | 147.09135 | 161.10696 | 8.39 |
| | Dapsone | C₁₂H₁₂N₂O₂S | M + H | 249.06922 | 156.01122 | 108.04431 | 9.03 |
| | Flunixin meglumine | C₁₄H₁₁F₃N₂O₂ | M + H | 297.08454 | 279.07397 | 264.0505 | 13.66 |
| | Acetaminophen | C₈H₉NO₂ | M + H | 152.07061 | 110.06004 | / | 6.25 |
| Psychoactive | Haloperidol | C₂₁H₂₃ClFNO₂ | M + H | 376.14741 | 358.13685 | 165.07102 | 10.3 |
| drugs | Imipramine | C₁₉H₂₄N₂ | M + H | 281.20123 | 86.0963 | 236.1434 | 10.71 |
| | Nitrazepam | C₁₅H₁₁N₃O₃ | M + H | 282.08732 | 268.08383 | 207.09126 | 11.53 |
| | Oxazepam | C₁₅H₁₁ClN₂O₂ | M + H | 287.05818 | 241.05203 | 269.04703 | 11.49 |
| | Pemoline | C₉H₈N₂O₂ | M + H | 177.06585 | 106.0651 | 79.0542 | 7.74 |
| | Perphenazine | C₂₁H₂₆ClN₃OS | M + H | 404.15579 | 143.11763 | 171.14877 | 10.83 |
| | Promethazine | C₁₇H₂₀N₂S | M + H | 285.142 | 198.0372 | 86.09643 | 10.34 |
| | Propionylpromazine | C₂₀H₂₄N₂OS | M + H | 341.16821 | 86.0964 | 58.0651 | 10.95 |
| | hydrochloride | | | | | | |
| | Sulpiride | C₁₅H₂₃N₃O₄S | M + H | 342.1482 | 214.0169 | 112.1121 | 6.21 |
| | Xylazine hydrochloride | C₁₂H₁₆N₂S | M + H | 221.1107 | 90.0372 | 164.0528 | 8.27 |
| | Acepromazin | C₁₉H₂₂N₂OS | M + H | 327.15256 | 254.06341 | 86.09643 | 10.2 |
| | Anisopirol | C₁₉H₂₄FN₃O | M + H | 330.19762 | 312.1865 | 121.076 | 7.44 |
| | Azaperone | C₁₉H₂₂FN₃O | M + H | 328.18197 | 165.07069 | 121.07603 | 8.04 |
| | Carbamazepine | C₁₅H₁₂N₂O | M + H | 237.10224 | 194.0964 | 220.0757 | 11.15 |
| | Chloropromazine | C₁₇H₁₉ClN₂S | M + H | 319.10302 | 86.09643 | 246.01379 | 11.25 |
| | Diazepam | C₁₆H₁₃ClN₂O | M + H | 285.07892 | 154.0415 | 193.0883 | 13.58 |
| | Diphenhydramine | C₁₇H₂₁NO | M + H | 256.16959 | 167.0855 | 152.0621 | 10.03 |
| | hydrochloride | | | | | | |
| | Droperidol | C₂₂H₂₂FN₃O₂ | M + H | 380.17688 | 165.07072 | 194.09726 | 9.35 |
| | Estazolam | C₁₆H₁₁ClN₄ | M + H | 295.0745 | 267.05524 | 205.0755 | 11.44 |
| Macrolides | Clarithromycin | C₃₈H₆₉NO₁₃ | M + H | 748.48417 | 158.11756 | 116.07061 | 11.04 |
| | Tiamulin | C₂₈H₄₇NO₄S | M + H | 494.32986 | 192.10528 | 119.01613 | 10.8 |
| | Erythromycin | C₃₇H₆₇NO₁₃ | M + H | 734.46852 | 158.11756 | 116.07061 | 9.92 |
| | Kitasamycin | C₄₀H₆₇NO₁₄ | M + H | 786.46343 | 558.32727 | 174.11247 | 11.25 |
| | Tilmicosin | C₄₆H₈₀N₂O₁₃ | M + H | 869.57332 | 174.11247 | 88.07569 | 8.93 |
| | Tylosin | C₄₆H₇₇NO₁₇ | M + H | 916.52643 | 772.44778 | 174.11247 | 10.17 |
| anthelmintics | Mebendazole | C₁₆H₁₃N₃O₃ | M + H | 296.10297 | 264.07675 | 105.03349 | 10.85 |
| | Mebendazole-amine | C₁₄H₁₁N₃O | M + H | 238.09749 | 105.03369 | / | 8.45 |
| | 2-aminofluorobenzimidazole | C₁₄H₁₀FN₃O | M + H | 256.08807 | 123.0241 | 113.0397 | 8.74 |
| | Oxfendazole | C₁₅H₁₃N₃O₃S | M + H | 316.07504 | 284.04882 | 223.0576 | 9.26 |
| | Oxibendazole | C₁₂H₁₅N₃O₃ | M + H | 250.11862 | 218.0924 | 176.04545 | 9.33 |
| | Parbendazole | C₁₃H₁₇N₃O₂ | M + H | 248.13935 | 216.1131 | 160.0505 | 10.65 |
| | 5-hydroxythiabendazole | C₁₀H₇N₃OS | M + H | 218.03826 | 191.02736 | / | 6.44 |
| | Tiabendazole | C₁₀H₇N₃S | M + H | 202.04334 | 175.03245 | 131.06037 | 7.12 |
| | Albendazole | C₁₂H₁₅N₃O₂S | M + H | 266.09577 | 234.06956 | 191.01478 | 10.99 |
| | Albendazole sulfone | C₁₂H₁₅N₃O₄S | M + H | 298.0856 | 266.05939 | 224.01244 | 9.4 |
| | Albendazole sulfoxide | C₁₂H₁₅N₃O₃S | M + H | 282.09069 | 240.04347 | 208.01752 | 8.08 |
| | Amino albendazole | C₁₀H₁₃N₃O₂S | M + H | 240.08012 | 198.03317 | 165.05328 | 6.94 |
| | sulfone | | | | | | |
| | Benzimidazole | C₇H₆N₂ | M + H | 119.06037 | 92.0495 | 65.0386 | 4.8 |
| | Febantel | C₂₀H₂₂N₄O₆S | M + H | 447.13328 | 383.08085 | 280.05391 | 14.78 |
| | Lobendazole | C₁₀H₁₁N₃O₂ | M + H | 206.0924 | 160.0505 | 178.0609 | 7.59 |
| | Toltrazuril sulfoxide | C₁₈H₁₄F₃N₃O₅S | M − H | 440.05335 | 371.05813 | / | 8.97 |
| | Clopidol | C₇H₇Cl₂NO | M + H | 191.99775 | 86.9996 | 101.01525 | 6.83 |
| | Dinitolmide | C₈H₇N₃O₅ | M − H | 224.03129 | 181.02438 | 77.03858 | 9.91 |
| | Halofuginone | C₁₆H₁₇BrClN₃O₃ | M + H | 414.02146 | 100.07598 | 120.08076 | 8.89 |
| | Levamisole | C₁₁H₁₂N₂S | M + H | 205.0794 | 178.0685 | / | 6.99 |
| Pesticides | Buprofezin | C₁₆H₂₃N₃OS | M + H | 306.16346 | 201.10561 | 116.05285 | 17.73 |
| | Carboxin | C₁₂H₁₃NO₂S | M + H | 236.07398 | 143.01613 | 93.0573 | 12.61 |
| | 3-hydroxycarbofuran | C₁₂H₁₅NO₄ | M + H | 238.10738 | 163.07536 | 135.08044 | 9.09 |
| | Clothianidin | C₆H₈ClN₅O₂S | M + H | 250.016 | 131.96692 | 113.0168 | 8.96 |
| | Cyromazine | C₆H₁₀N₆ | M + H | 167.10397 | 85.05087 | 125.08217 | 3.63 |
| | Diuron | C₉H₁₀Cl₂N₂O | M + H | 233.02429 | 72.04439 | 159.97153 | 12.65 |
| | Ethopabate | C₁₂H₁₅NO₄ | M + H | 238.10738 | 206.08117 | 164.07061 | 10.29 |
| | Fipronil | C₁₂H₄Cl₂F₆N₄OS | M − H | 434.93143 | 329.95845 | 249.95853 | 15.66 |
| | Fluridone | C₁₉H₁₄F₃NO | M + H | 330.11003 | 310.1038 | 290.09757 | 13.62 |
| | Acephate | C₄H₁₀NO₃PS | M + H | 184.01918 | 142.99264 | 112.99968 | 5.61 |
| | Hexazinone | C₁₂H₂₀N₄O₂ | M + H | 253.1659 | 171.08765 | 71.06037 | 10.59 |
| | Imazalil | C₁₄H₁₄Cl₂N₂O | M + H | 297.0556 | 158.97628 | 255.00864 | 10.77 |
| | Imidacloprid | C₉H₁₀ClN₅O₂ | M + H | 256.05958 | 209.05885 | 175.09793 | 9.3 |
| | Linuron | C₉H₁₀Cl₂N₂O₂ | M + H | 249.01921 | 159.97153 | 182.02414 | 14.09 |
| | Metribuzin | C₈H₁₄N₄OS | M + H | 215.09611 | 187.10119 | 131.03859 | 11.73 |
| | Myclobutanil | C₁₅H₁₇ClN₄ | M + H | 289.12145 | 70.03997 | 125.01525 | 14.21 |
| | Acetamiprid | C₁₀H₁₁ClN₄ | M + H | 223.0745 | 126.0105 | 98.9997 | 9.58 |
| | Norflurazon | C₁₂H₉ClF₃N₃O | M + H | 304.0459 | 284.03967 | 160.03686 | 12.81 |
| | Propyzamide | C₁₂H₁₁C₁₂NO | M + H | 256.02905 | 189.9821 | 172.95555 | 14.68 |
| | Atrazine | C₈H₁₄ClN₅ | M + H | 216.10105 | 174.0541 | 96.05562 | 12.63 |
| | Simazine | C₇H₁₂ClN₅ | M + H | 202.0854 | 132.0323 | 104.001 | 11.35 |
| | Desethyl atrazine | C₆H₁₀ClN₅ | M + H | 188.06975 | 146.0228 | 104.001 | 9.37 |
| | Azoxystrobin | C₂₂H₁₇N₃O₅ | M + H | 404.1241 | 344.10397 | 329.07949 | 14.2 |
| | Benoxacor | C₁₁H₁₁Cl₂NO₂ | M + H | 260.02396 | 149.08352 | 120.04439 | 14.31 |
| Other | Scopolamine | C₁₇H₂₁NO₄ | M + H | 304.15433 | 156.10191 | 138.09134 | 7 |
| substances | Valnemulin | C₃₁H₅₂N₂O₅S | M + H | 565.36697 | 263.1417 | 72.08078 | 11.15 |
| | Atropine | C₁₇H₂₃NO₃ | M + H | 290.17507 | 124.11208 | 93.06988 | 7.61 |

(6) analyzing the filtrate by the ultra high performance liquid chromatography-quadrupole/electrostatic field orbitrap high resolution mass spectrometer to obtain original data of the sample;

(7) subjecting the original data of the sample to automatic extraction using Trace Finder software within a mass window of 5 ppm; wherein when a compound meets the following conditions at the same time, it indicates that the compound is a suspected positive compound in the sample:

(i) a signal-noise ratio (S/N) of the compound is greater than 3;

(ii) a difference between the measured retention time of the compound and the retention time of the compound in the mass spectrometry database is equal to or less than 0.2 min or within ±2.5% (not to exceed 0.5 min);

(iii) the measured parent ion and one fragment ion of the compound simultaneously match the information in the mass spectrometry database, and a mass accuracy of the parent ion is less than 5 ppm and a mass accuracy of the fragment ion is less than 10 ppm; and

(8) comparing a fragment ion mass spectrometry of the suspected positive compound in the sample with a fragment ion mass spectrometry of standard for further identification; wherein if the mass accuracy between main fragment ions in the two fragment ion mass spectrometry is less than 10 ppm and the main fragment ions in the two fragment ion mass spectrometries have identical relative abundance, it confirms that the suspected positive compound is present in the sample.

In an embodiment, in step (3), when the sample is muscle or poultry egg, the extracting solution is a mixed solution of 3.0 mL of Mcllvaine-Na₂EDTA buffered solution and 10 mL of acetonitrile, where a concentration of EDTA in the Mcllvaine-Na₂EDTA buffered solution is 0.1 mol/L; when the sample is cow's milk or goat's milk, the extracting solution is 20 mL of acetonitrile.

In an embodiment, in step (4), the solid phase extraction column is a HyperSep Retain PEP column.

In an embodiment, in steps (5) and (6), parameters of the ultra high performance liquid chromatography are set as follows: chromatographic column: C18 (150 mm×3.0 mm, particle size 1.8 m); column temperature: 30° C.; injection volume: 10 μL; flow rate: 0.3 mL/min; mobile phase A: 0.1% aqueous formic acid solution for positive ion mode and 0.03% aqueous ammonia solution for negative ion mode; mobile phase B: a solution of 0.1% formic acid in acetonitrile for positive ion mode and a solution of 0.03% ammonia in acetonitrile for negative ion mode; a gradient elution program in positive ion mode is shown in Table 2, and a gradient elution program in negative ion mode is shown in Table 3.

TABLE 2
Gradient elution program in positive ion mode

| Time (min) | A(%) | B(%) |
|---|---|---|
| 0 | 95 | 5 |
| 1.0 | 95 | 5 |
| 15 | 5 | 95 |
| 17 | 5 | 95 |
| 17.1 | 95 | 5 |
| 22.0 | 95 | 5 |

TABLE 3
Gradient elution program in negative ion mode

| Time (min) | A(%) | B(%) |
|---|---|---|
| 0 | 95 | 5 |
| 8 | 5 | 95 |
| 10 | 5 | 95 |
| 10.1 | 95 | 5 |
| 13 | 95 | 5 |

In an embodiment, in steps (5) and (6), parameters of the mass spectrometry are set as follows:

ion source: electron spray ion source (ESI+ and ESI−);

scan mode: full scan and automatically-triggered secondary scan mode;

capillary voltage: 3.2 kv (ESI+) and 2.8 kv (ESI−);

ion transmission capillary temperature: 325° C.;

atomizing gas temperature: 350° C.;

sheath gas pressure: 40 arb;

auxiliary gas pressure: 40 arb;

dwell time of the primary mass spectrometry: 100 ms;

dwell time of the secondary mass spectrometry: 60 ms;

scanning range (m/z): 50-1000; and

collision energy: 20, 40 and 60 eV.

In an embodiment, in step (3), the shaking is performed at 6,000 rpm for 10 min; and the centrifugation is performed at 4,000-7,000 rpm for 5 min.

Compared with the prior art, the invention has the following beneficial effects.

1. The invention facilitates collecting high-quality and high-accuracy data, reducing the false positive rate.

2. The sample is directly analyzed in full scan mode, and there is no limit to the number of compounds scanned per unit time. Moreover, it is convenient to establish a method for the instrumental analysis.

3. The invention enables the rapid screening of compounds in the sample by comparing the measured data with the mass spectrometry database, and is particularly suitable for the screening and confirmation of multiple types of compounds.

4. The invention is suitable for the detection of veterinary drugs and additives in animal-derived food, which enriches the detection and screening technical means for multiple residues in animal-derived food, facilitating ensuring the safety of animal husbandry and food industry.

### BRIEF DESCRIPTION OF THE DRAWINGS

FIG. 1 is a parent ion extraction chromatogram of enrofloxacin.

FIG. 2 is a parent ion mass spectrometry of the enrofloxacin.

FIG. 3 is a fragment ion mass spectrometry of the enrofloxacin.

FIG. 4 is a parent ion chromatogram of sulfamethazine.

FIG. 5 is a parent ion chromatogram of sulformethoxine.

FIG. 6 is a parent ion chromatogram of sulfisoxazole.

FIG. 7 is a parent ion chromatogram of sulfaguanidine.

FIG. 8 is a parent ion chromatogram of sulfamerazine.

FIG. 9 is a parent ion chromatogram of sulfamethylthiadizaole.

FIG. 10 is a parent ion chromatogram of sulfamethoxazole.

FIG. 11 is a parent ion chromatogram of sulfamethoxydiazine.

FIG. 12 is a parent ion chromatogram of sulfamethoxypyridazine.

FIG. 13 is a parent ion chromatogram of sulfamonomethoxine.

FIG. 14 is a parent ion chromatogram of sulfamethazole.

FIG. 15 is a parent ion chromatogram of sulfapyridinee.

FIG. 16 is a parent ion chromatogram of sulfaquinoxaline.

FIG. 17 is a parent ion chromatogram of sulfathiazole.

FIG. 18 is a parent ion chromatogram of sulfisomidine.

FIG. 19 is a parent ion chromatogram of trimethoprim.

FIG. 20 is a parent ion chromatogram of diaveridine.

FIG. 21 is a parent ion chromatogram of phthalylsulfathiazole.

FIG. 22 is a parent ion chromatogram of sulfabenzamide.

FIG. 23 is a parent ion chromatogram of sulfacetamide.

FIG. 24 is a parent ion chromatogram of sulfachlorpyridazine.

FIG. 25 is a parent ion chromatogram of sulfachloropyrazine. FIG. 26 is a parent ion chromatogram of sulfadiazine.

FIG. 27 is a parent ion chromatogram of sulfadimethoxine.

FIG. 28 is a parent ion chromatogram of lomefloxacin.

FIG. 29 is a parent ion chromatogram of marbofloxacin.

FIG. 30 is a parent ion chromatogram of nadifloxacin.

FIG. 31 is a parent ion chromatogram of nalidixic acid.

FIG. 32 is a parent ion chromatogram of norfloxacin.

FIG. 33 is a parent ion chromatogram of ofloxacin.

FIG. 34 is a parent ion chromatogram of orbifloxacin.

FIG. 35 is a parent ion chromatogram of oxolinic acid.

FIG. 36 is a parent ion chromatogram of pefloxacin.

FIG. 37 is a parent ion chromatogram of pipemidic acid.

FIG. 38 is a parent ion chromatogram of cinoxacin.

FIG. 39 is a parent ion chromatogram of sarafloxacin.

FIG. 40 is a parent ion chromatogram of sparfloxacin.

FIG. 41 is a parent ion chromatogram of ciprofloxacin.

FIG. 42 is a parent ion chromatogram of danofloxacin.

FIG. 43 is a parent ion chromatogram of difloxacin.

FIG. 44 is a parent ion chromatogram of enoxacin.

FIG. 45 is a parent ion chromatogram of enrofloxacin.

FIG. 46 is a parent ion chromatogram of fleroxacin.

FIG. 47 is a parent ion chromatogram of flumequine.

FIG. 48 is a parent ion chromatogram of gatifloxacin.

FIG. 49 is a parent ion chromatogram of mebendazole.

FIG. 50 is a parent ion chromatogram of mebendazole-amine.

FIG. 51 is a parent ion chromatogram of 2-aminofluorobenzimidazole.

FIG. 52 is a parent ion chromatogram of oxfendazole.

FIG. 53 is a parent ion chromatogram of oxibendazole.

FIG. 54 is a parent ion chromatogram of parbendazole.

FIG. 55 is a parent ion chromatogram of 5-hydroxythiabendazole. FIG. 56 is a parent ion chromatogram of tiabendazole.

FIG. 57 is a parent ion chromatogram of albendazole.

FIG. 58 is a parent ion chromatogram of albendazole sulfone.

FIG. 59 is a parent ion chromatogram of albendazole sulfoxide.

FIG. 60 is a parent ion chromatogram of amino albendazole sulfone.

FIG. 61 is a parent ion chromatogram of benzimidazole.

FIG. 62 is a parent ion chromatogram of febantel.

FIG. 63 is a parent ion chromatogram of lobendazole.

FIG. 64 is a parent ion chromatogram of penbutolol.

FIG. 65 is a parent ion chromatogram of phenylethanolamine A.

FIG. 66 is a parent ion chromatogram of ractopamine.

FIG. 67 is a parent ion chromatogram of salbutamol.

FIG. 68 is a parent ion chromatogram of salmeterol.

FIG. 69 is a parent ion chromatogram of terbutaline.

FIG. 70 is a parent ion chromatogram of mabuterol.

FIG. 71 is a parent ion chromatogram of tulobuterol.

FIG. 72 is a parent ion chromatogram of bambuterol.

FIG. 73 is a parent ion chromatogram of brombuterol.

FIG. 74 is a parent ion chromatogram of cimaterol.

FIG. 75 is a parent ion chromatogram of cimbuterol.

FIG. 76 is a parent ion chromatogram of clenbuterol.

FIG. 77 is a parent ion chromatogram of clenproperol.

FIG. 78 is a parent ion chromatogram of clonidine.

FIG. 79 is a parent ion chromatogram of cyproheptadine.

FIG. 80 is a parent ion chromatogram of fenoterol.

FIG. 81 is a parent ion chromatogram of labetalol.

FIG. 82 is a parent ion chromatogram of betamethasone dipropionate.

FIG. 83 is a parent ion chromatogram of betamethasone valerate.

FIG. 84 is a parent ion chromatogram of chlormadinone acetate.

FIG. 85 is a parent ion chromatogram of 17a-methyltestosterone. FIG. 86 is a parent ion chromatogram of clobetasol propionate.

FIG. 87 is a parent ion chromatogram of clobetasone butyrate.

FIG. 88 is a parent ion chromatogram of corticosterone.

FIG. 89 is a parent ion chromatogram of hydrocortisone.

FIG. 90 is a parent ion chromatogram of cortisone acetate.

FIG. 91 is a parent ion chromatogram of cortisone.

FIG. 92 is a parent ion chromatogram of deflazacort.

FIG. 93 is a parent ion chromatogram of dexamethasone.

FIG. 94 is a parent ion chromatogram of diflorasone diacetate.

FIG. 95 is a parent ion chromatogram of epitestosterone.

FIG. 96 is a parent ion chromatogram of estradiol benzoate.

FIG. 97 is a parent ion chromatogram of fludrocortisone acetate.

FIG. 98 is a parent ion chromatogram of flumetasone pivalate.

FIG. 99 is a parent ion chromatogram of flumethasone.

FIG. 100 is a parent ion chromatogram of fluocinolone acetonide.

FIG. 101 is a parent ion chromatogram of fluorometholone.

FIG. 102 is a parent ion chromatogram of flurandrenolide.

FIG. 103 is a parent ion chromatogram of fluticasone propionate.

FIG. 104 is a parent ion chromatogram of nandrolone propionate.

FIG. 105 is a parent ion chromatogram of halcinonide.

FIG. 106 is a parent ion chromatogram of dihydrodiethylstilbestrol.

FIG. 107 is a parent ion chromatogram of medroxyprogesterone acetate.

FIG. 108 is a parent ion chromatogram of megestrol acetate.

FIG. 109 is a parent ion chromatogram of melengestrol acetate.

FIG. 110 is a parent ion chromatogram of methandienone.

FIG. 111 is a parent ion chromatogram of methylprednisolone.

FIG. 112 is a parent ion chromatogram of norethindrone.

FIG. 113 is a parent ion chromatogram of nandrolone (19-nortestosterone).

FIG. 114 is a parent ion chromatogram of levonorgestrel.

FIG. 115 is a parent ion chromatogram of prednicarbate. FIG. 116 is a parent ion chromatogram of prednisolone.

FIG. 117 is a parent ion chromatogram of prednisone.

FIG. 118 is a parent ion chromatogram of progesterone.

FIG. 119 is a parent ion chromatogram of dehydrotestosterone.

FIG. 120 is a parent ion chromatogram of testosterone.

FIG. 121 is a parent ion chromatogram of trenbolone.

FIG. 122 is a parent ion chromatogram of triamcinolone acetonide FIG. 123 is a parent ion chromatogram of beclomethasone.

FIG. 124 is a parent ion chromatogram of betamethasone.

FIG. 125 is a parent ion chromatogram of dimetridazole.

FIG. 126 is a parent ion chromatogram of ipronidazole.

FIG. 127 is a parent ion chromatogram of metronidazole.

FIG. 128 is a parent ion chromatogram of ornidazole.

FIG. 129 is a parent ion chromatogram of hydroxydimetridazole.

FIG. 130 is a parent ion chromatogram of ronidazole.

FIG. 131 is a parent ion chromatogram of secnidazole.

FIG. 132 is a parent ion chromatogram of tinidazole.

FIG. 133 is a parent ion chromatogram of amantadine.

FIG. 134 is a parent ion chromatogram of rimantadine.

FIG. 135 is a parent ion chromatogram of clindamycin.

FIG. 136 is a parent ion chromatogram of lincomycin.

FIG. 137 is a parent ion chromatogram of chloramphenicol.

FIG. 138 is a parent ion chromatogram of florfenicol.

FIG. 139 is a parent ion chromatogram of thiamphenicol.

FIG. 140 is a parent ion chromatogram of ceftiofur.

FIG. 141 is a parent ion chromatogram of flucloxacillin.

FIG. 142 is a parent ion chromatogram of cloxacillin.

FIG. 143 is a parent ion chromatogram of nafcillin.

FIG. 144 is a parent ion chromatogram of piperacillin.

FIG. 145 is a parent ion chromatogram of penicillin G. FIG. 146 is a parent ion chromatogram of quinocetone.

FIG. 147 is a parent ion chromatogram of phenacetin.

FIG. 148 is a parent ion chromatogram of salicylic acid.

FIG. 149 is a parent ion chromatogram of amidopyrine.

FIG. 150 is a parent ion chromatogram of antipyrine.

FIG. 151 is a parent ion chromatogram of dapsone.

FIG. 152 is a parent ion chromatogram of flunixin meglumine.

FIG. 153 is a parent ion chromatogram of acetaminophen.

FIG. 154 is a parent ion chromatogram of oxazepam.

FIG. 155 is a parent ion chromatogram of pemoline.

FIG. 156 is a parent ion chromatogram of perphenazine.

FIG. 157 is a parent ion chromatogram of promethazine.

FIG. 158 is a parent ion chromatogram of propionylpromazine hydrochloride.

FIG. 159 is a parent ion chromatogram of sulpiride.

FIG. 160 is a parent ion chromatogram of xylazine hydrochloride.

FIG. 161 is a parent ion chromatogram of acepromazin.

FIG. 162 is a parent ion chromatogram of anisopirol.

FIG. 163 is a parent ion chromatogram of azaperone.

FIG. 164 is a parent ion chromatogram of carbamazepine.

FIG. 165 is a parent ion chromatogram of chloropromazine.

FIG. 166 is a parent ion chromatogram of diazepam.

FIG. 167 is a parent ion chromatogram of diphenhydramine hydrochloride.

FIG. 168 is a parent ion chromatogram of droperidol.

FIG. 169 is a parent ion chromatogram of haloperidol.

FIG. 170 is a parent ion chromatogram of imipramine.

FIG. 171 is a parent ion chromatogram of nitrazepam.

FIG. 172 is a parent ion chromatogram of estazolam.

FIG. 173 is a parent ion chromatogram of clarithromycin.

FIG. 174 is a parent ion chromatogram of tiamulin.

FIG. 175 is a parent ion chromatogram of erythromycin. FIG. 176 is a parent ion chromatogram of kitasamycin.

FIG. 177 is a parent ion chromatogram of tilmicosin.

FIG. 178 is a parent ion chromatogram of tylosin.

FIG. 179 is a parent ion chromatogram of toltrazuril sulfocide.

FIG. 180 is a parent ion chromatogram of clopidol.

FIG. 181 is a parent ion chromatogram of dinitolmide.

FIG. 182 is a parent ion chromatogram of halofuginone.

FIG. 183 is a parent ion chromatogram of levamisole.

FIG. 184 is a parent ion chromatogram of carboxin.

FIG. 185 is a parent ion chromatogram of 3-hydroxycarbofuran.

FIG. 186 is a parent ion chromatogram of a primary parent ion of a chromatogram of clothianidin.

FIG. 187 is a parent ion chromatogram of cyromazine.

FIG. 188 is a parent ion chromatogram of diuron.

FIG. 189 is a parent ion chromatogram of ethopabate.

FIG. 190 is a parent ion chromatogram of fipronil.

FIG. 191 is a parent ion chromatogram of fluridone.

FIG. 192 is a parent ion chromatogram of acephate.

FIG. 193 is a parent ion chromatogram of hexazinone.

FIG. 194 is a parent ion chromatogram of imazalil.

FIG. 195 is a parent ion chromatogram of imidacloprid.

FIG. 196 is a parent ion chromatogram of linuron.

FIG. 197 is a parent ion chromatogram of metribuzin.

FIG. 198 is a parent ion chromatogram of myclobutanil.

FIG. 199 is a parent ion chromatogram of acetamiprid.

FIG. 200 is a parent ion chromatogram of norflurazon.

FIG. 201 is a parent ion chromatogram of propyzamide.

FIG. 202 is a parent ion chromatogram of atrazine.

FIG. 203 is a parent ion chromatogram of simazine.

FIG. 204 is a parent ion chromatogram of desethyl atrazine. FIG. 205 is a parent ion chromatogram of azoxystrobin.

FIG. 206 is a parent ion chromatogram of benoxacor.

FIG. 207 is a parent ion chromatogram of buprofezin.

FIG. 208 is a parent ion chromatogram of scopolamine.

FIG. 209 is a parent ion chromatogram of valnemulin.

FIG. 210 is a parent ion chromatogram of atropine.

### DETAILED DESCRIPTION OF EMBODIMENTS

Provided herein was a screening and confirmation method for veterinary drugs and additives in animal-derived food, where the veterinary drugs and additives were selected from:

(a) sulfonamides: sulfamethazine, sulformethoxine, sulfisoxazole, sulfaguanidine, sulfamerazine, sulfamethylthiadizaole, sulfamethoxazole, sulfamethoxydiazine, sulfamethoxypyridazine, sulfamonomethoxine, sulfamethazole, sulfapyridinee, sulfaquinoxaline, sulfathiazole, sulfisomidine, trimethoprim, diaveridine, phthalylsulfathiazole, sulfabenzamide, sulfacetamide, sulfachlorpyridazine, sulfachloropyrazine, sulfadiazine and sulfadimethoxine;

(b) fluoroquinolones: lomefloxacin, marbofloxacin, nadifloxacin, nalidixic acid, norfloxacin, ofloxacin, orbifloxacin, oxolinic acid, pipemidic acid, pefloxacin, cinoxacin, sarafloxacin, sparfloxacin, ciprofloxacin, danofloxacin, difloxacin, enoxacin, enrofloxacin, fleroxacin, flumequine and gatifloxacin;

(c) benzimidazoles: mebendazole, mebendazole-amine, 2-aminofluorobenzimidazole, oxfendazole, oxibendazole, parbendazole, 5-hydroxythiabendazole, tiabendazole, albendazole, albendazole sulfone, albendazole sulfoxide, amino albendazole sulfone, benzimidazole, febantel and lobendazole;

(d) stimulants: cyproheptadine, fenoterol, labetalol, penbutolol, phenylethanolamine A, ractopamine, salbutamol, salmeterol, terbutaline, mabuterol, tulobuterol, bambuterol, brombuterol, cimaterol, cimbuterol, clenbuterol, clenproperol and clonidine;

(e) hormones: chlormadinone acetate, 17a-methyltestosterone, clobetasol propionate, clobetasone butyrate, corticosterone, hydrocortisone, cortisone acetate, cortisone, deflazacort, dexamethasone, diflorasone diacetate, epitestosterone, estradiol benzoate, fludrocortisone acetate, flumetasone pivalate, flumethasone, fluocinolone acetonide, fluorometholone, flurandrenolide, fluticasone propionate, nandrolone propionate, halcinonide, dihydrodiethylstilbestrol, medroxyprogesterone acetate, megestrol acetate, melengestrol acetate, methandienone, methylprednisolone, norethindrone, nandrolone propionate, levonorgestrel, prednicarbate, prednisolone, prednisone, progesterone, dehydrotestosterone, testosterone, trenbolone, triamcinolone acetonide, beclomethasone, betamethasone dipropionate, betamethasone valerate and betamethasone;

(f) nitroimidazoles: dimetridazole, ipronidazole, metronidazole, ornidazole, hydroxydimetridazole, ronidazole, secnidazole and tinidazole;

(g) antiviral agents: amantadine and rimantadine;

(h) lincosamide: clindamycin and lincomycin;

(i) amphenicols: chloramphenicol, florfenicol and thiamphenicol;

(j) penicillins: ceftiofur, flucloxacillin, cloxacillin, nafcillin, piperacillin and penicillin G;

(k) quinoxalines: quinocetone;

(l) antipyretic and analgesics: phenacetin, salicylic acid, amidopyrine, antipyrine, dapsone, flunixin meglumine and acetaminophen;

(m) psychoactive agents: haloperidol, imipramine, nitrazepam, oxazepam, pemoline, perphenazine, promethazine, propionylpromazine hydrochloride, sulpiride, xylazine hydrochloride, acepromazin, anisopirol, azaperone, carbamazepine, chloropromazine, diazepam, diphenhydramine hydrochloride, droperidol, estazolam, clarithromycin, tiamulin, erythromycin, kitasamycin, tilmicosin and tylosin;

(n) anthelmintics: toltrazuril sulfocide, clopidol, dinitolmide, halofuginone and levamisole;

(o) pesticides: buprofezin, carboxin, 3-hydroxycarbofuran, clothianidin, cyromazine, diuron, ethopabate, fipronil, fluridone, acephate, hexazinone, imazalil, imidacloprid, linuron, metribuzin, myclobutanil, acetamiprid, norflurazon, propyzamide, atrazine, simazine, desethyl atrazine, azoxystrobin and benoxacor; and

(p) other substances: scopolamine, valnemulin and atropine.

The method was specifically described as follows.

### 1. Preparation of Standard Working Solutions

1.1 Preparation of Stock Solutions

10 mg of each reference standard was weighed accurately, dissolved and diluted with methanol to 10 mL in a volumetric flask to prepare a 1 mg/mL stock solution of each reference standard; where norfloxacin was dissolved with water; the pesticides were dissolved with acetone; diaveridine, albendazole, albendazole sulfone and febantel were all dissolved with formic acid and then diluted with methanol to 10 mL; and the rest substances with lower solubility were dissolved with a mixed solvent of methanol and ammonia water or a mixed solvent of methanol (main solvent) and dimethyl sulfoxide (auxiliary solvent), and then diluted to 10 mL.

1.2 Preparation of Standard Working Solutions

(1) 100 μL of the stock solution of each reference standard in the same category in Table 1 was accurately pipetted to a volumetric flask, and then diluted to 10 mL with methanol to accordingly prepare 16 first mixed standard solutions, where each reference standard in the corresponding first mixed standard solution thereof had a concentration of 10 μg/mL. 100 μL of each first mixed standard solution was accurately pipetted to another volumetric flask and then diluted to 10 mL with methanol to prepare a second mixed standard solution, where each reference standard in the second mixed standard solution had a concentration of 100 ng/mL. (2) accurately 100 μL, 500 μL, 1 mL and 5 mL of the second mixed standard solution were pipetted, respectively, and, then diluted to 10 mL with a 20% aqueous methanol solution to prepare a series of mixed standard working solutions, where each reference standard in the series of mixed standard working solutions had a concentration of 1 ng/mL, 5 ng/mL, 10 ng/mL and 50 ng/mL, respectively.

### 2. Pretreatment of Samples

2.1 Sample Preparation

2.1.1 Animal Tissues

An appropriate amount of fresh/thawed blank or test tissue was collected, minced and homogenized.

2.1.2 Cow's Milk and Goat's Milk

An appropriate amount of fresh/thawed blank or test cow's milk was collected and mixed uniformly.

2.1.3 Poultry Eggs

An appropriate number of fresh test eggs were collected, shelled and homogenized.

The above samples were all stored below −20° C.

2.2 Extraction

2.2.1 Muscle or Poultry Egg

5 g of the sample was added into a 50 mL centrifuge tube, to which a mixed solution of 3.0 mL Mcllvaine-Na₂EDTA buffered solution and 10 mL acetonitrile was added for extraction, where the Mcllvaine-Na₂EDTA buffered solution was prepared through steps of: dissolving 44.08 g of disodium hydrogen phosphate, 37.2 g of Na₂EDTA and 8.08 g of citric acid followed by diluting to 1000 mL to prepare 0.1 mol/L Mcllvaine-Na₂EDTA buffered solution. The centrifuge tube was subjected to vortex mixing, shook at 6,000 rpm for 10 min and centrifuged at 4,000 rpm for 5 min. The supernatant was transferred to another 50 mL centrifuge tube, and the precipitate was repeatedly treated with the extraction solution. The two supernatants were combined, added with 6 g of anhydrous sodium sulfate and 1.5 g of sodium chloride, mixed under vortex for 30 s, subjected to standing for 10 min and centrifuged at 7,000 rpm for 5 min, and the supernatant was collected for further use.

2.2.2 Cow's Milk or Goat's Milk

5 g of cow's milk or goat's milk was added into a 50 mL centrifuge tube, to which 20 mL of acetonitrile was added for extraction. The centrifuge tube was subjected to vortex mixing, shook at 6,000 rpm for 10 min and centrifuged at 7,000 rpm for 5 min, and the supernatant was transferred to another 50 mL centrifuge tube, added with 4 g of anhydrous sodium sulfate and 1 g of sodium chloride, mixed under vortex for 30 s, subjected to standing for 10 min and centrifuged at 7,000 rpm for 5 min. The supernatant was collected for further use.

2.3 Purification

The solid phase extraction column was activated and equilibraed sequentially with 4 mL of methanol and 4 mL of water and then rinsed with 2 mL of the supernatant prepared in step (2.2.1) or (2.2.2). The effluent was discarded, and then 5 mL of the supernatant was loaded to the solid phase extraction column. The effluent was collected by a centrifuge tube, and 4 mL of the effluent was accurately pipetted, dried at 40° C. in nitrogen flow, added with 200 μL of methanol, mixed under vortex for 10 s, added with 800 μL of pure water, mixed under vortex for 30 s and filtered through a 0.22 m nylon filter membrane. The filtrate was collected and stored for further use.

### 3. Instrumental Analysis

3.1 Liquid Chromatography

The gradient elution programs in positive and negative ion modes were respectively shown in Table 4 and Table 5.

TABLE 4
Gradient elution program in positive ion mode

| Time (min) | A(%) | B(%) |
|---|---|---|
| 0 | 90-95 | 10-5 |
| 1.0 | 90-95 | 10-5 |
| 15 | 5-10 | 95-10 |
| 17 | 5-10 | 95-90 |
| 17.1 | 95-90 | 5-10 |
| 22.0 | 95-90 | 5-10 |

TABLE 5
Gradient elution program in negative ion mode

| Time (min) | A(%) | B(%) |
|---|---|---|
| 0 | 90-95 | 10-5 |
| 8 | 5-10 | 95-90 |
| 10 | 5-10 | 95-90 |
| 10.1 | 95-90 | 5-10 |
| 13 | 95-90 | 5-10 |

3.2 Mass Spectrometry

Parameters of the mass spectrometry were set as follows: ion source: electron spray ion source (ESI+ and ESI−); capillary voltage: 3.2 kv (ESI+), 2.8 kv (ESI−); ion transmission capillary temperature: 325° C.; atomizing gas temperature: 350° C.; sheath gas pressure: 40 arb; auxiliary gas pressure: 40 arb; dwell time of the primary mass spectrometry: 100 ms; dwell time of the secondary mass spectrometry: 60 ms; scanning range (m/z): 50-1000; collision energy: 20, 40, 60 eV; scan mode: full scan and automatically-triggered secondary scan mode.

### 4. Establishment of Spectrometry Database

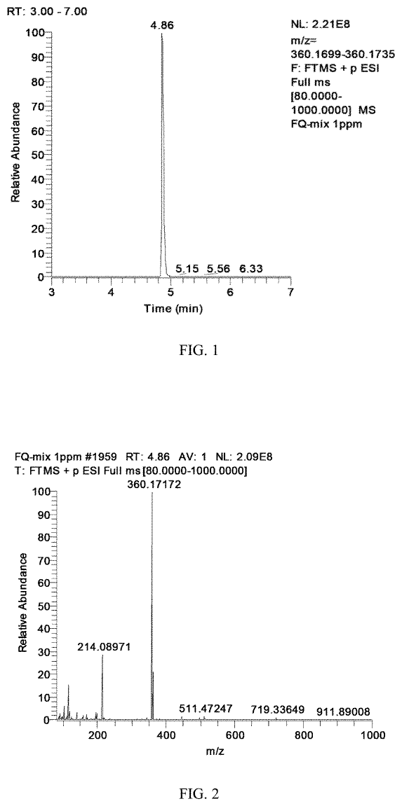

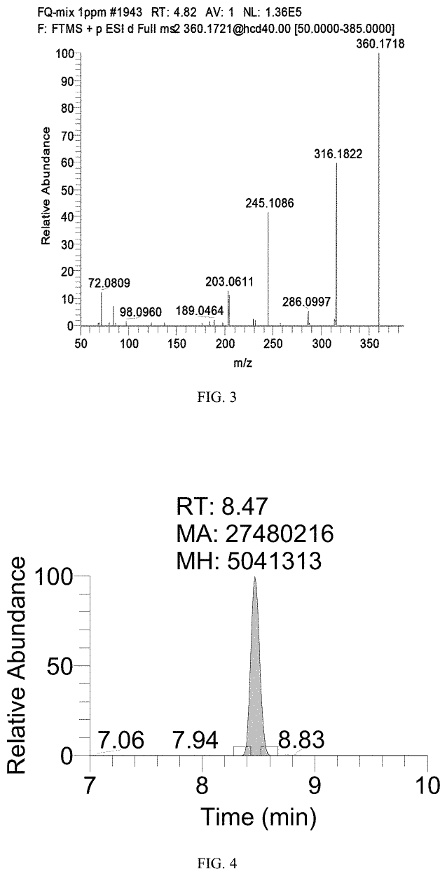

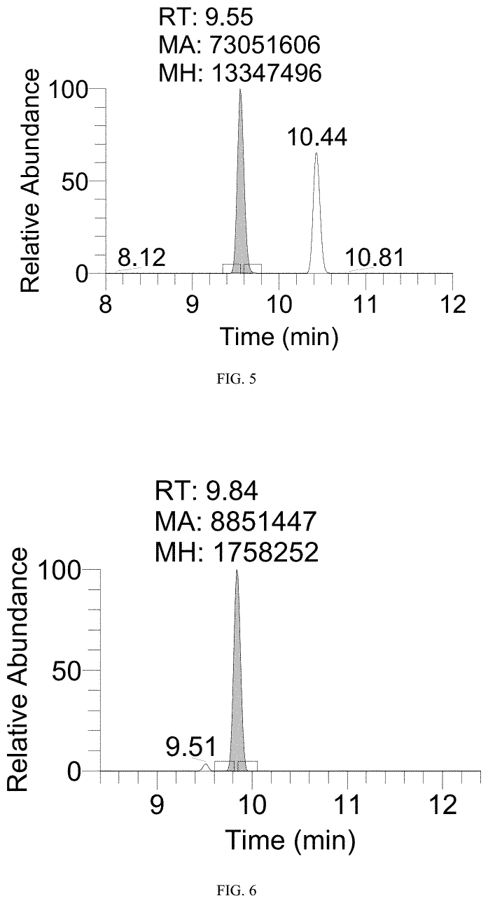

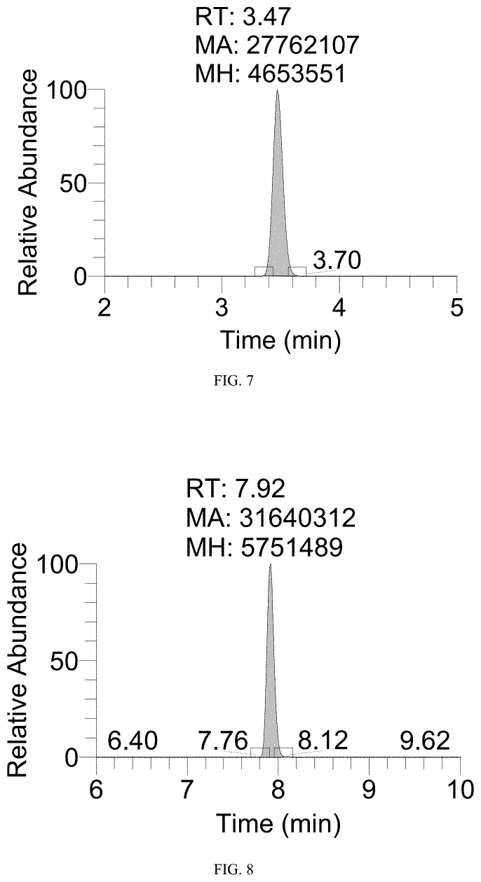

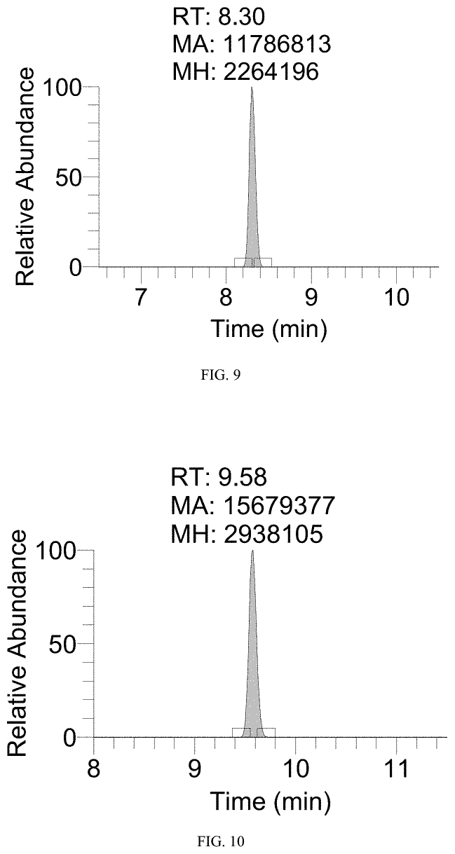

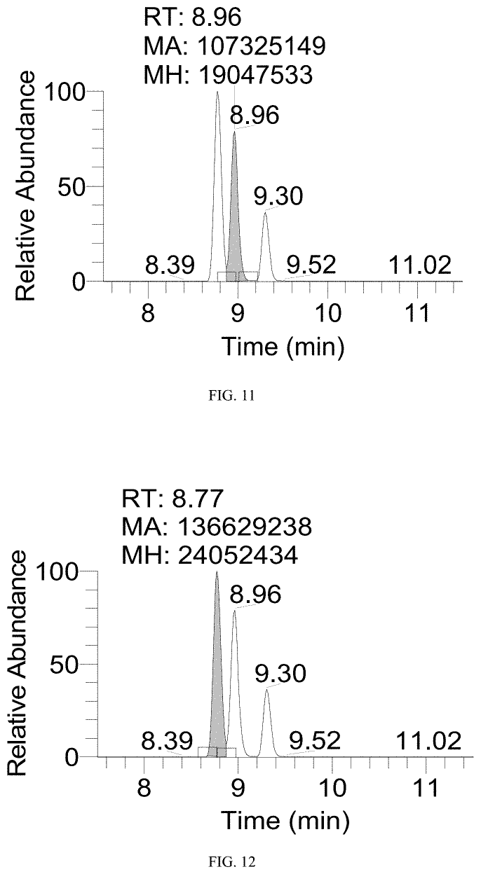

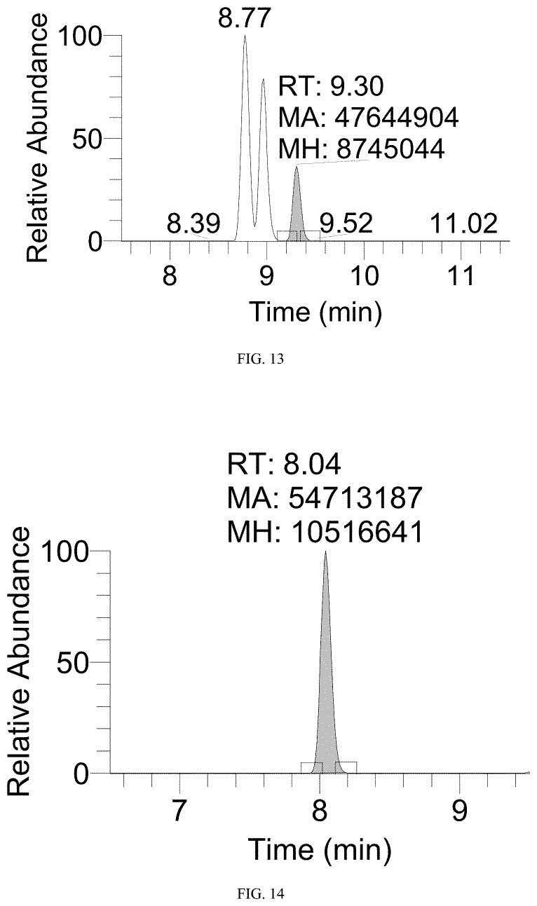

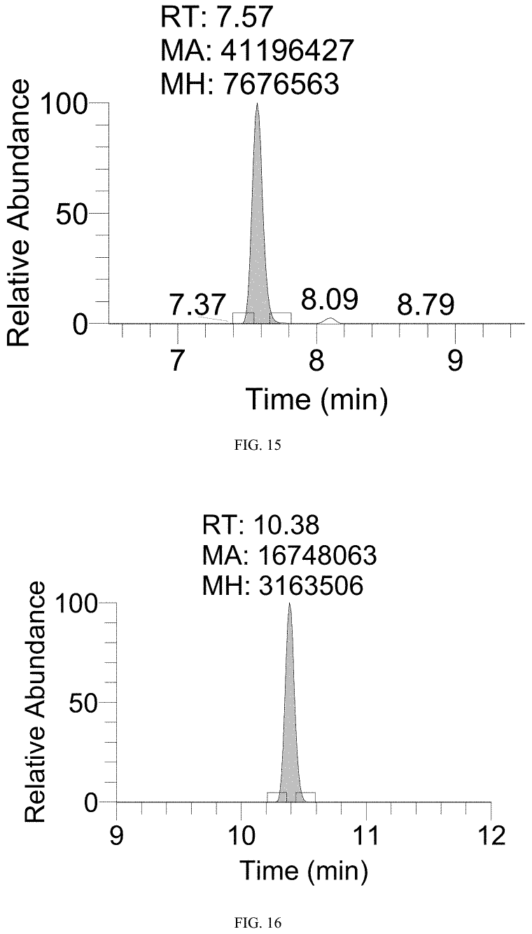

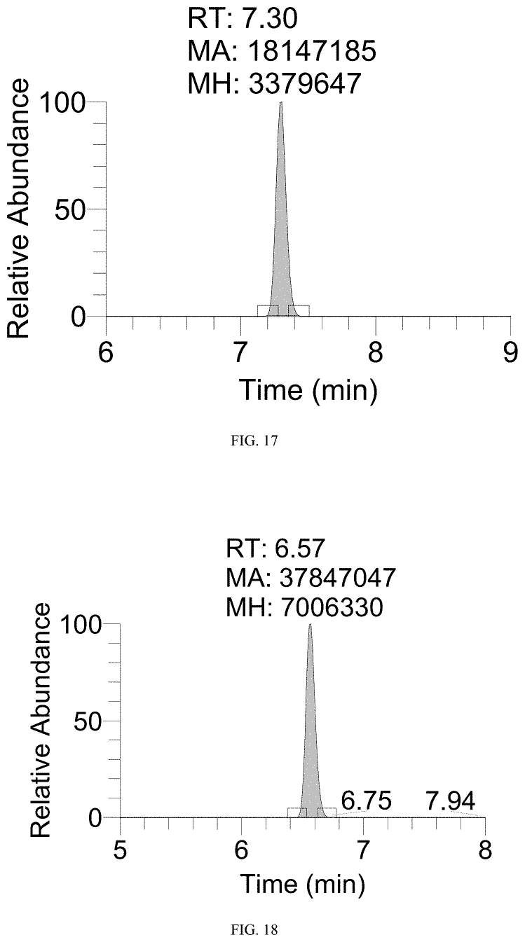

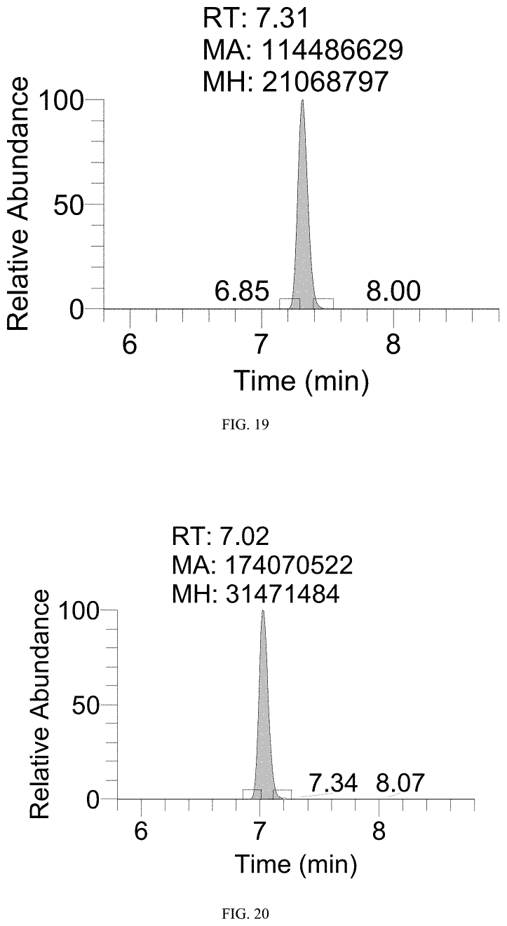

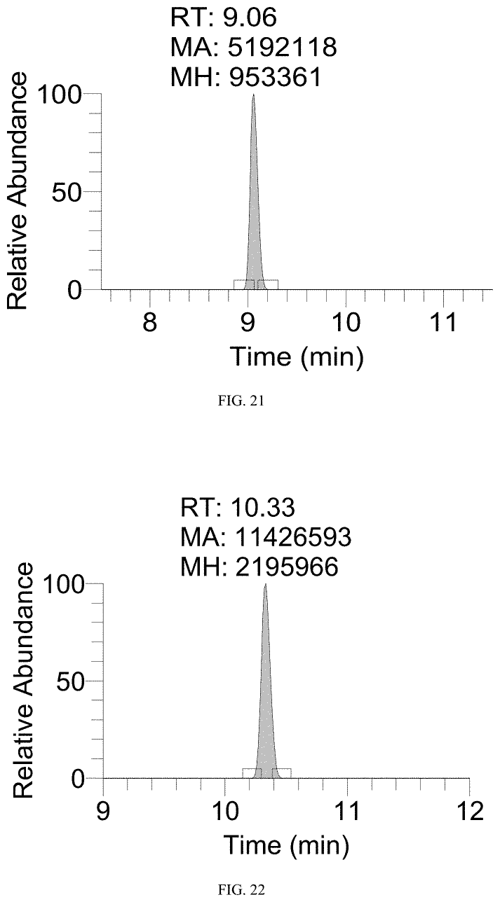

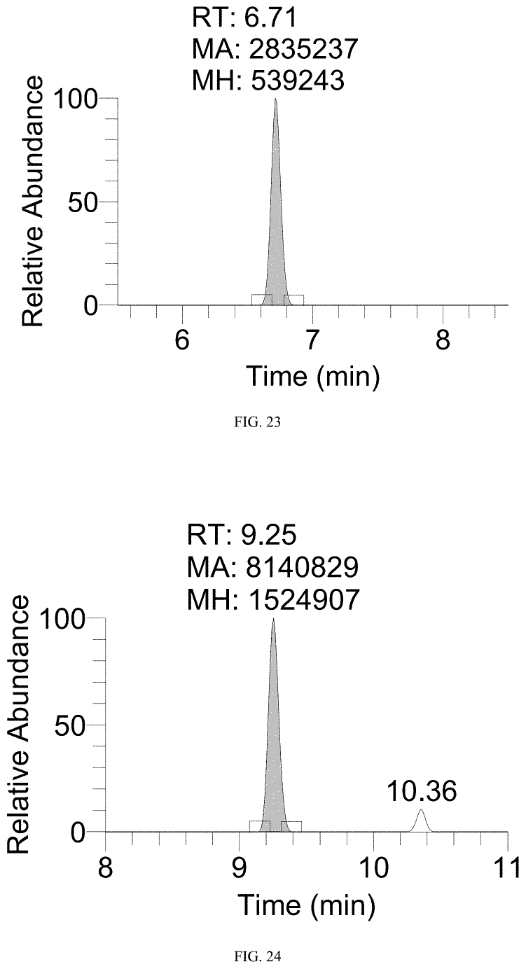

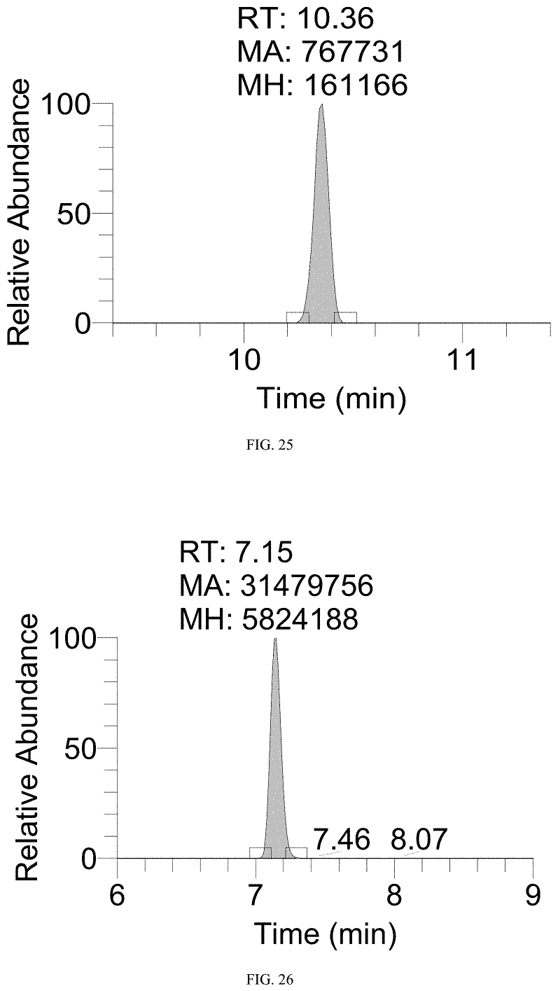

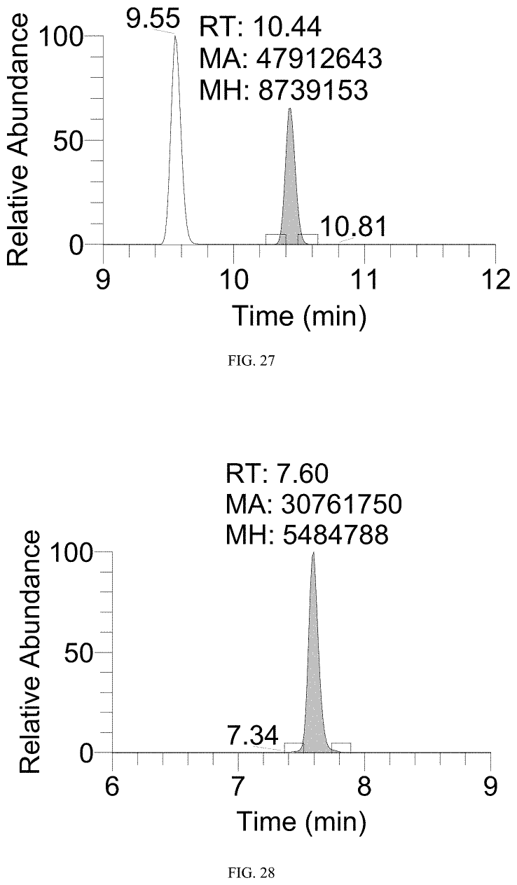

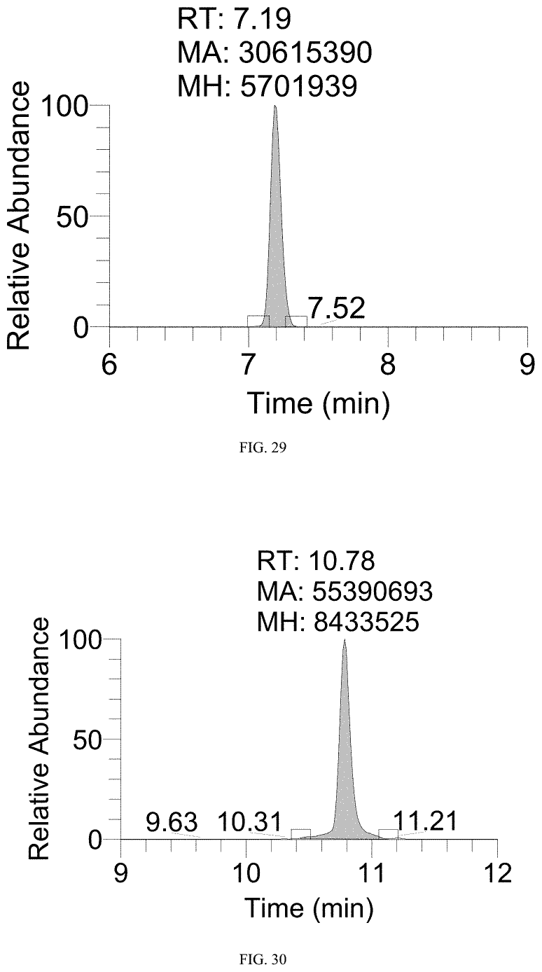

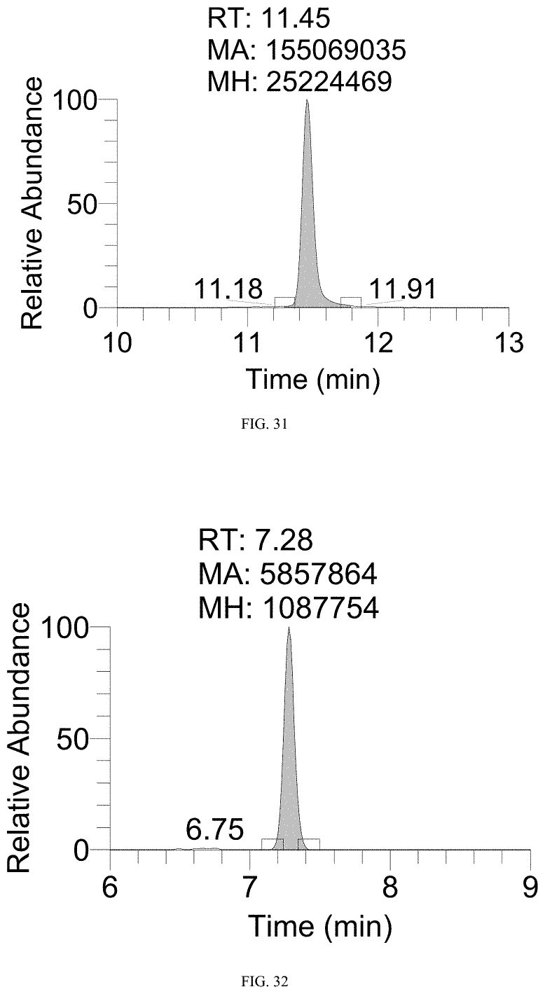

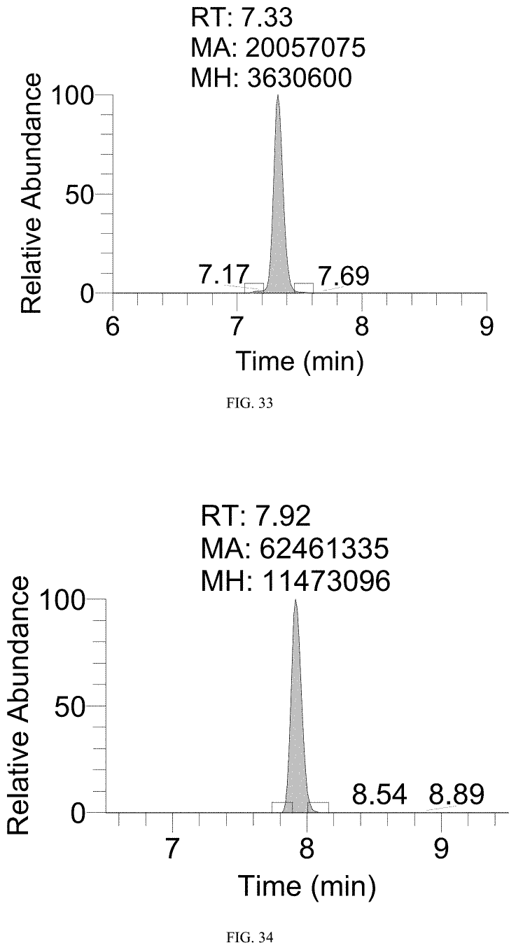

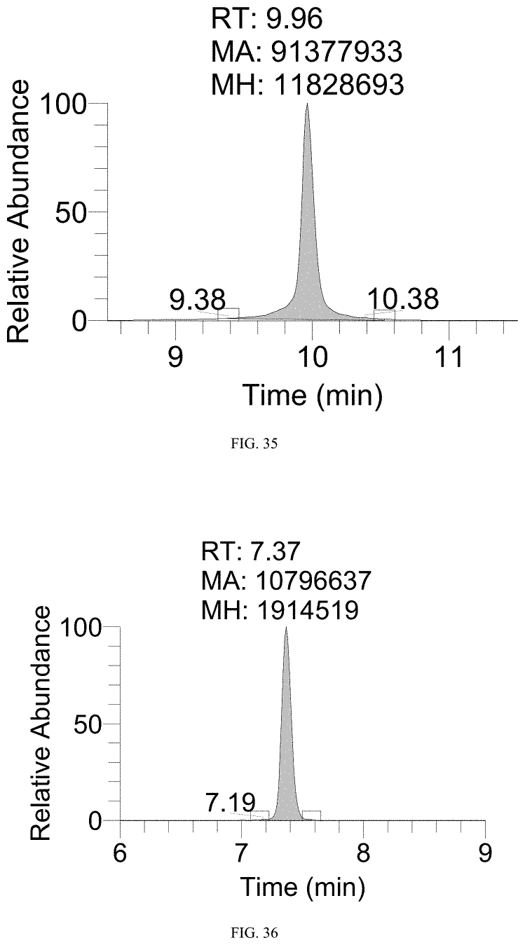

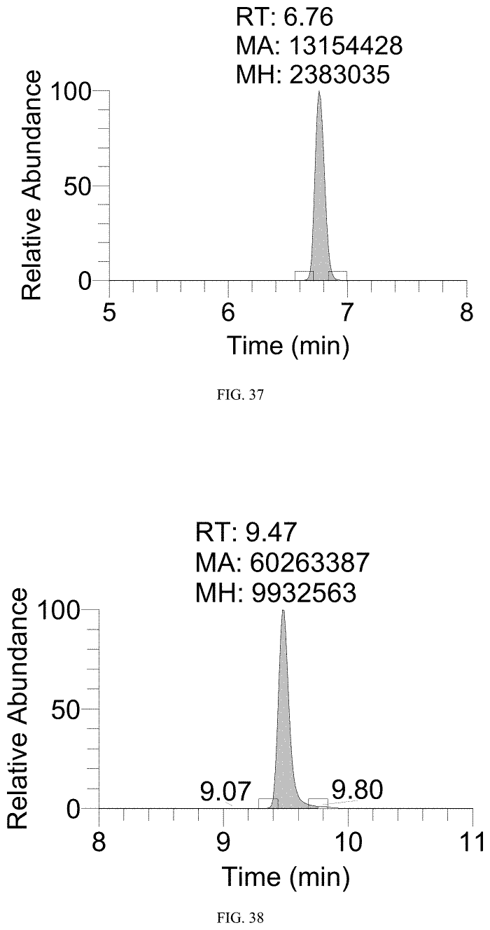

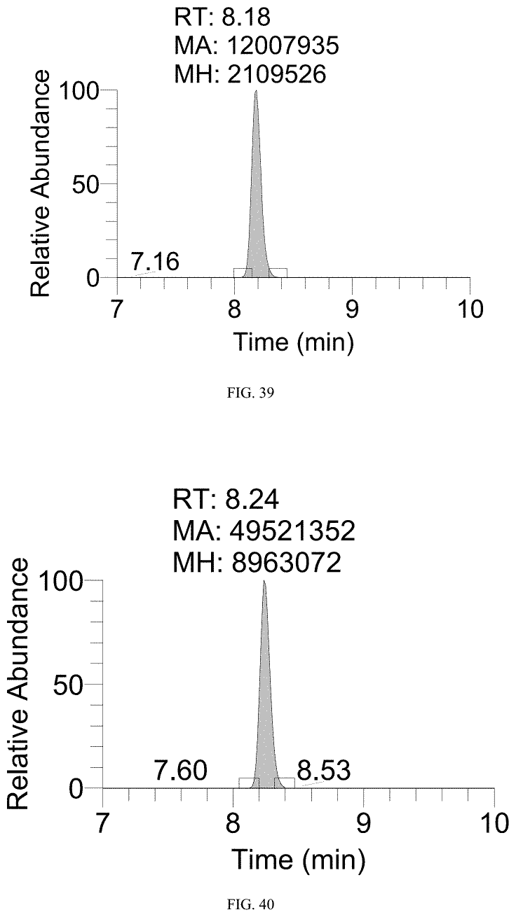

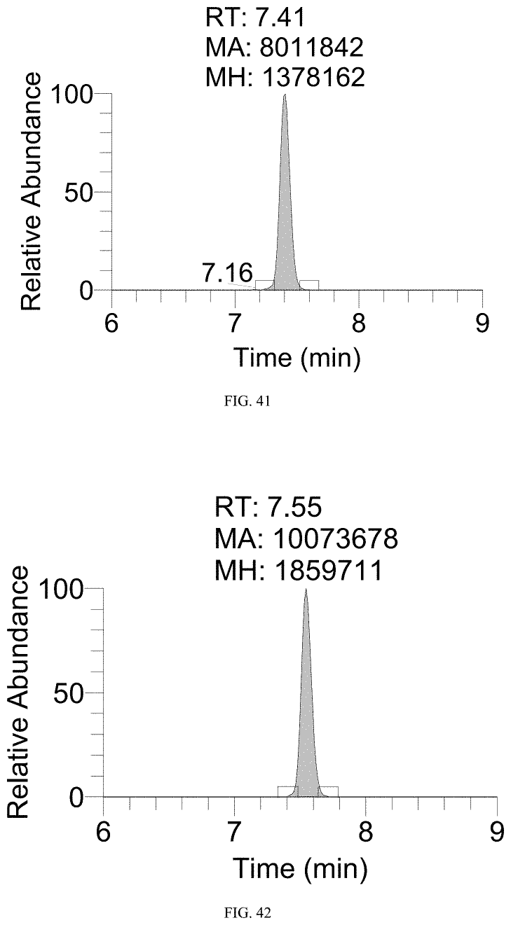

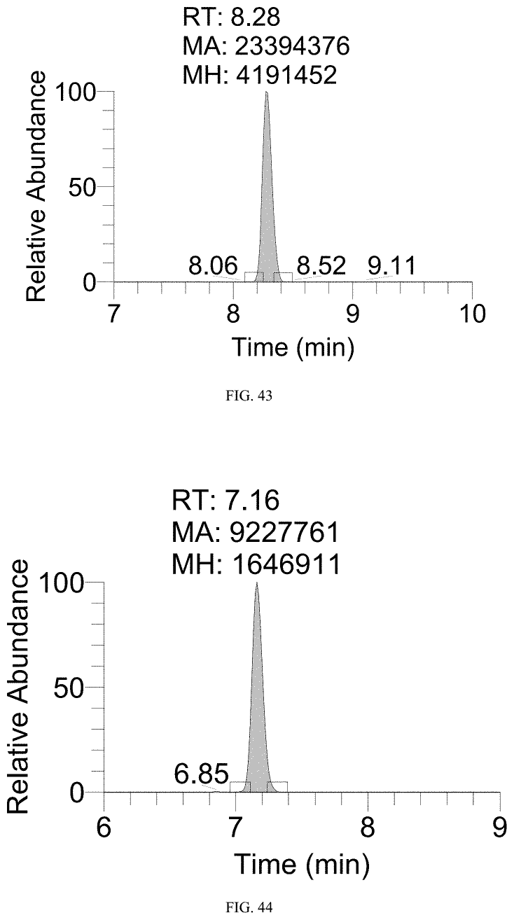

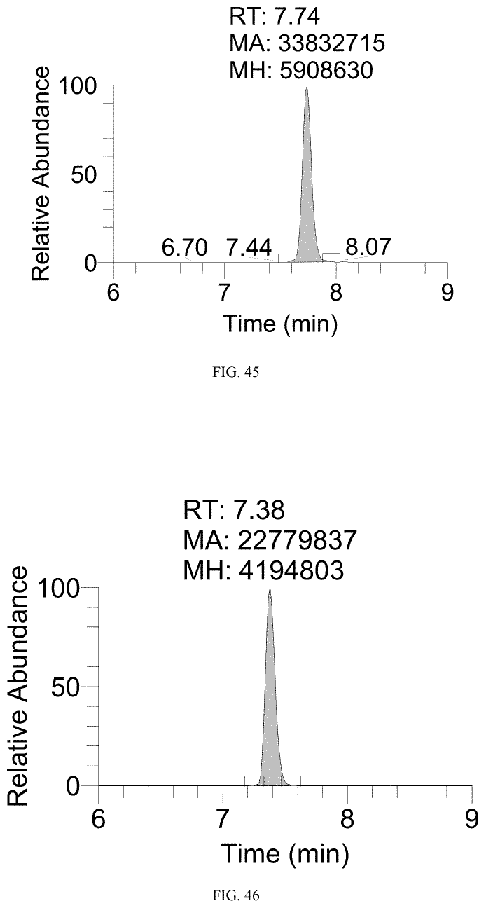

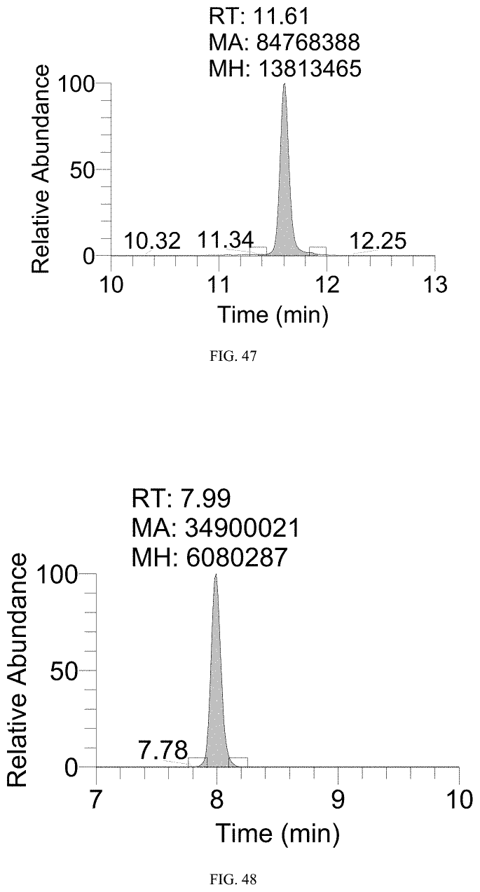

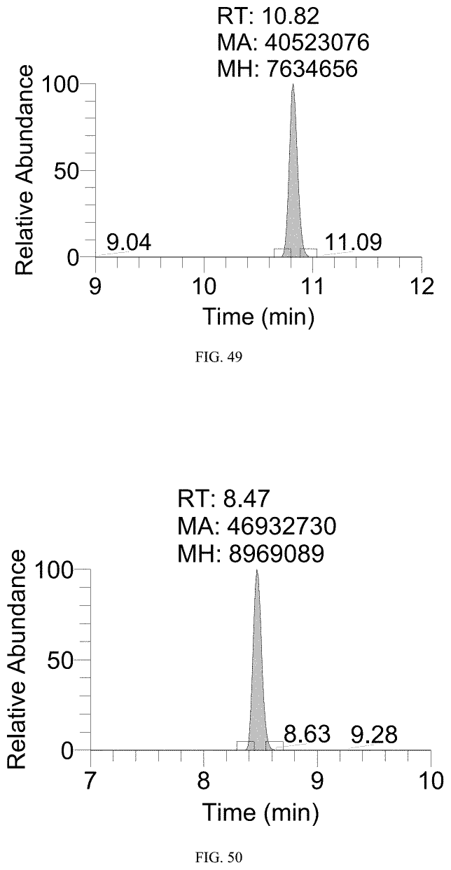

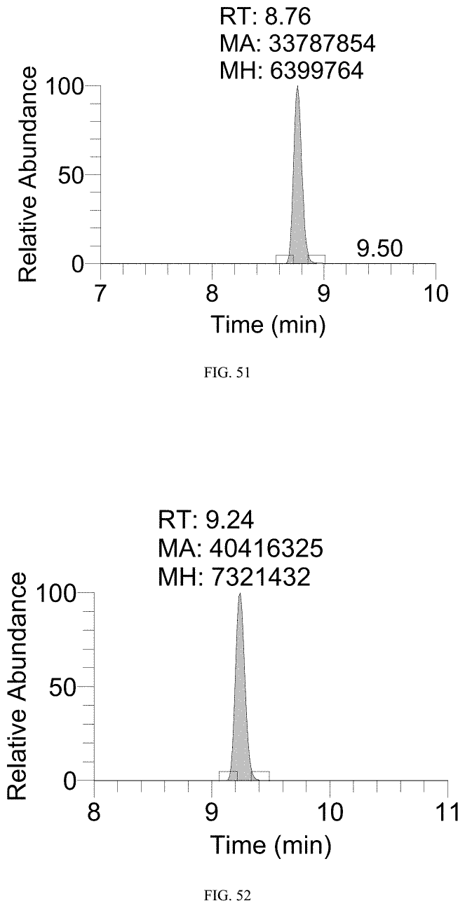

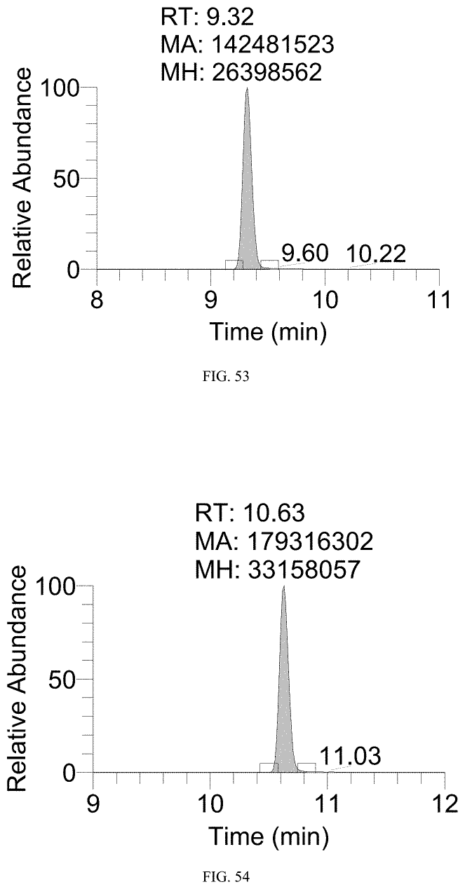

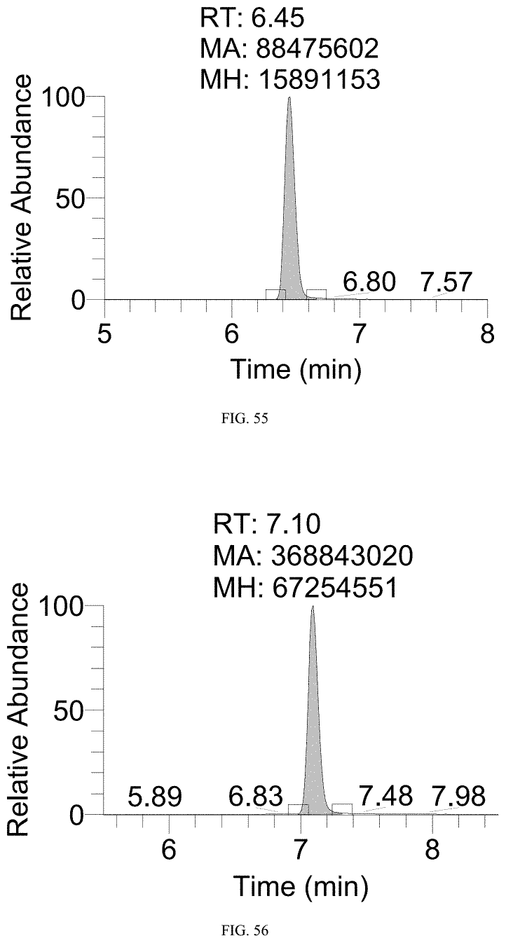

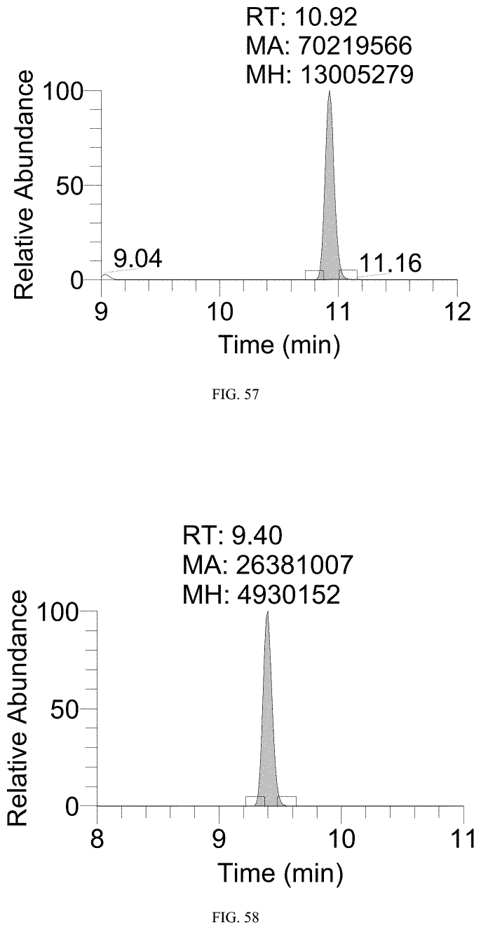

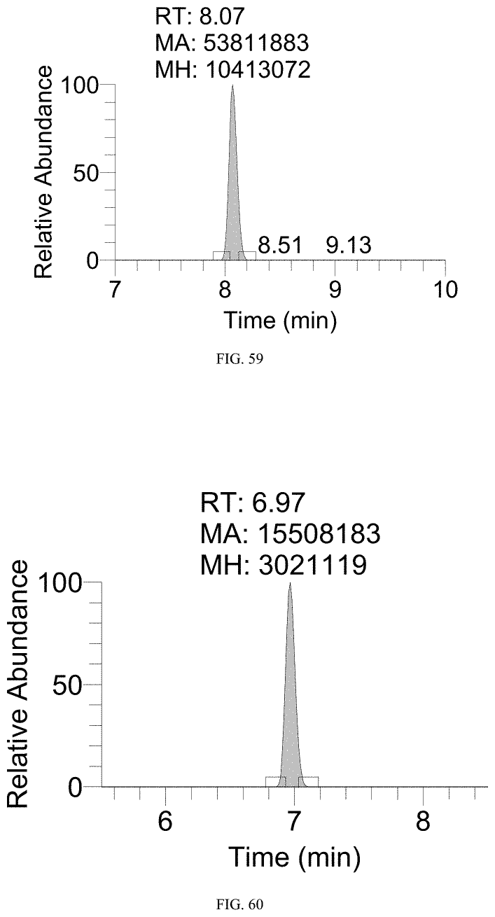

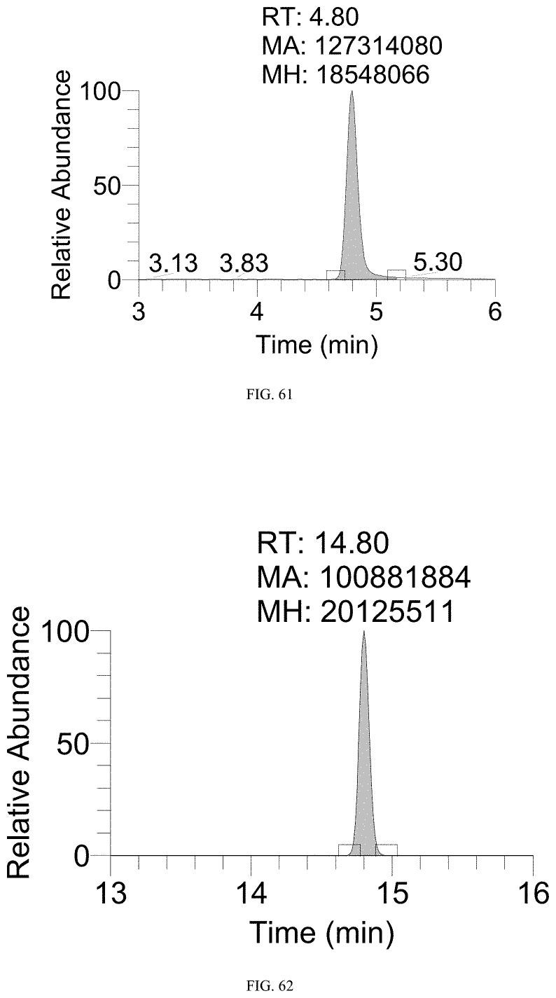

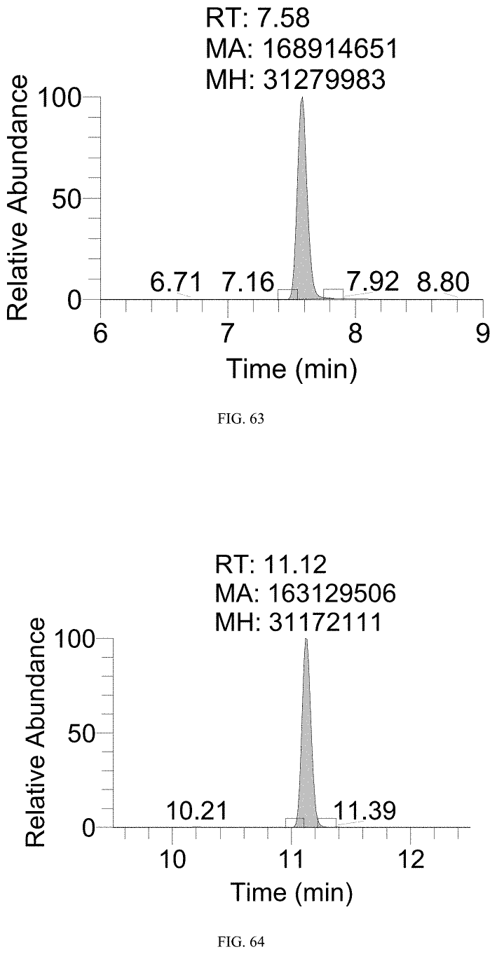

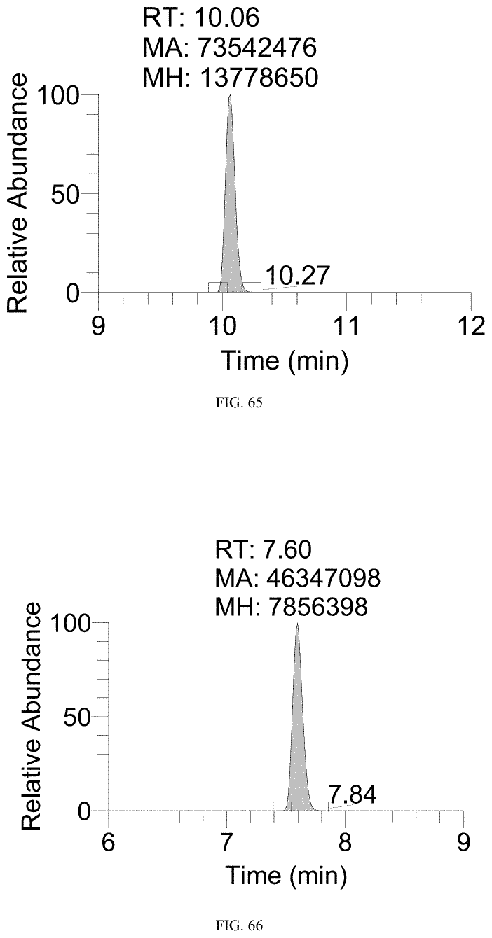

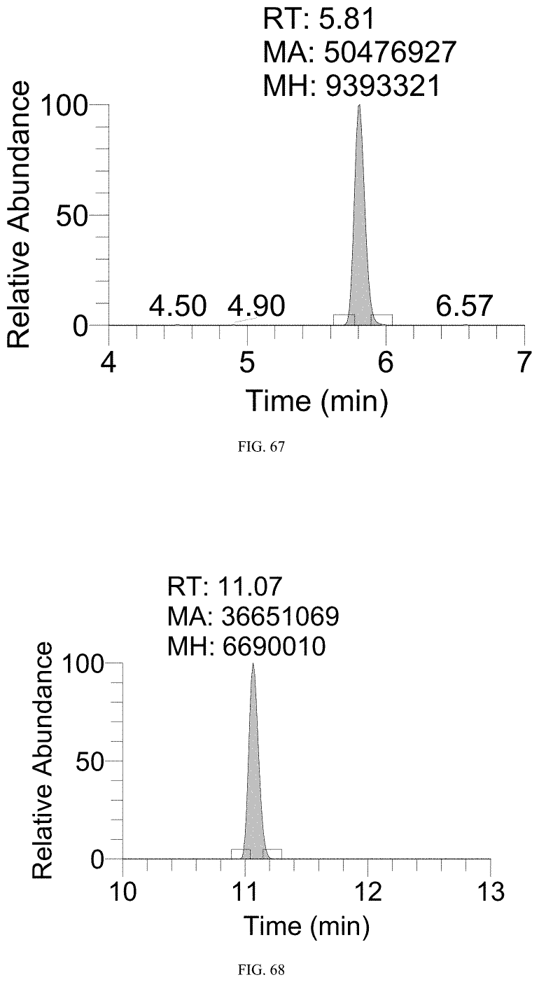

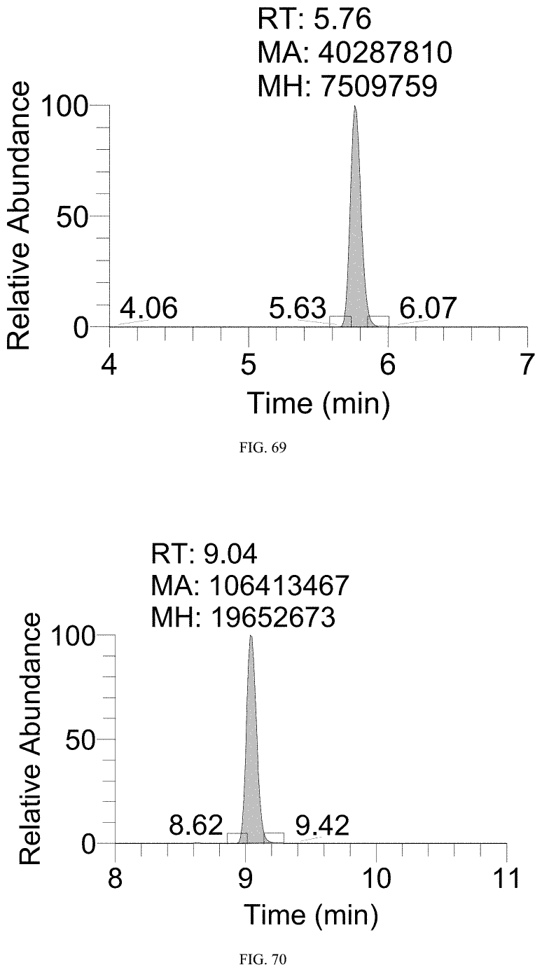

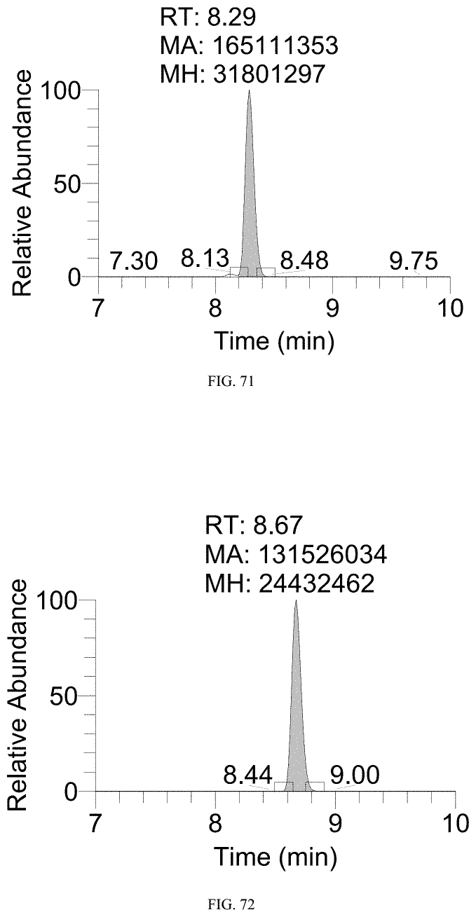

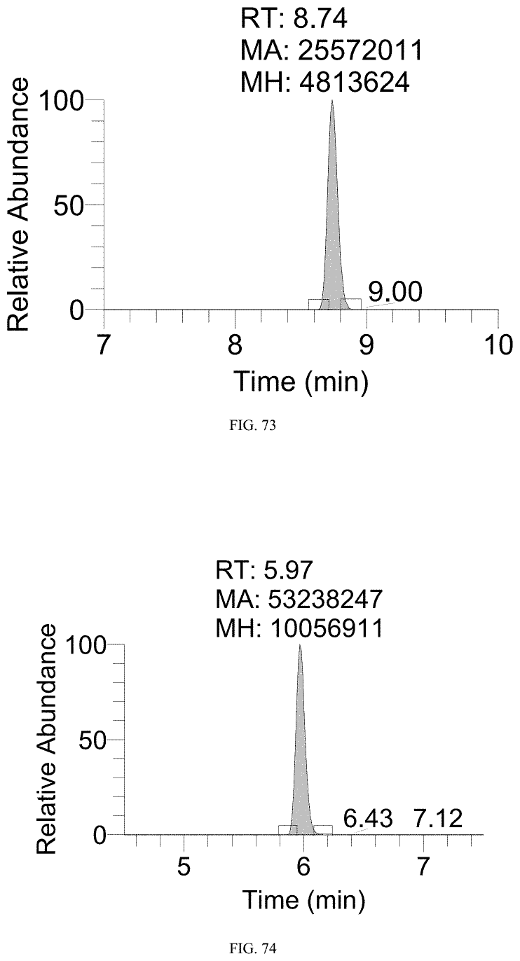

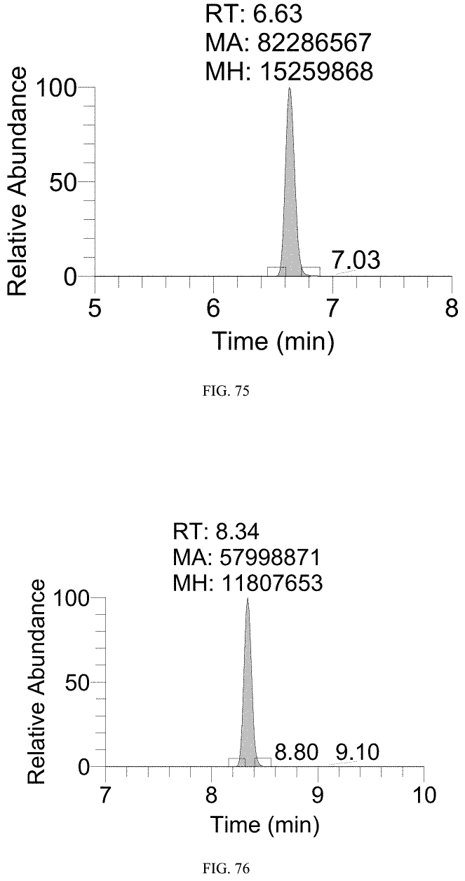

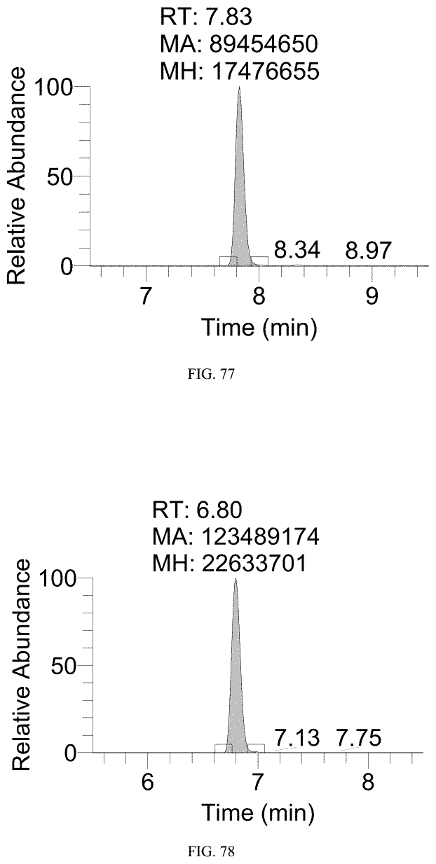

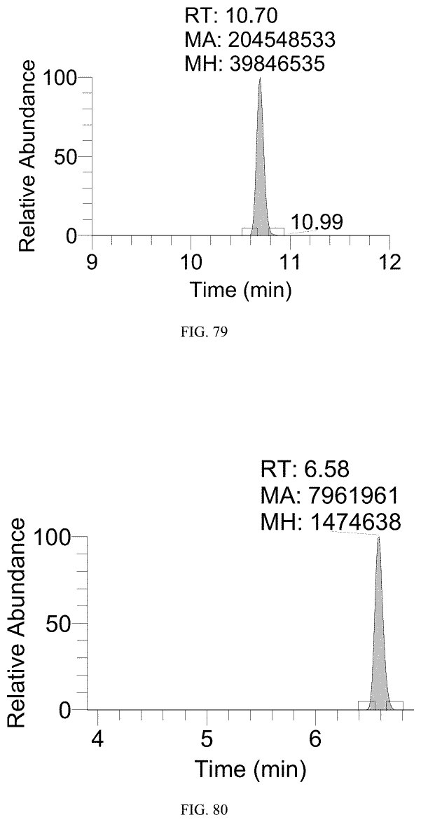

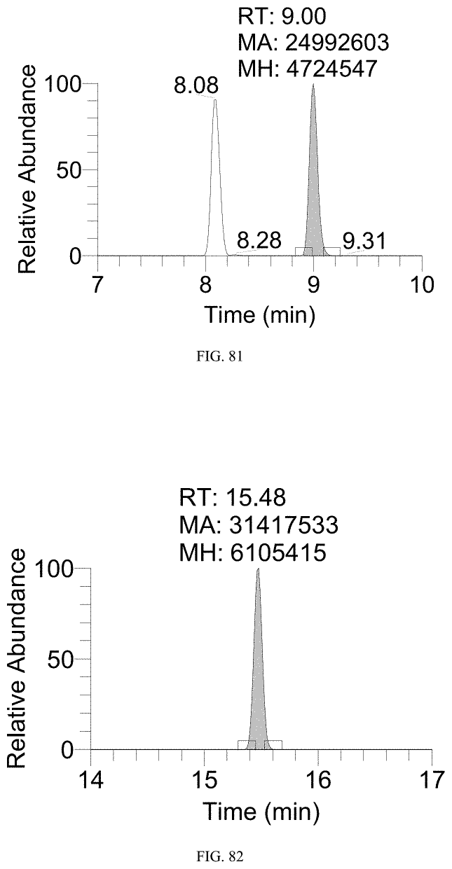

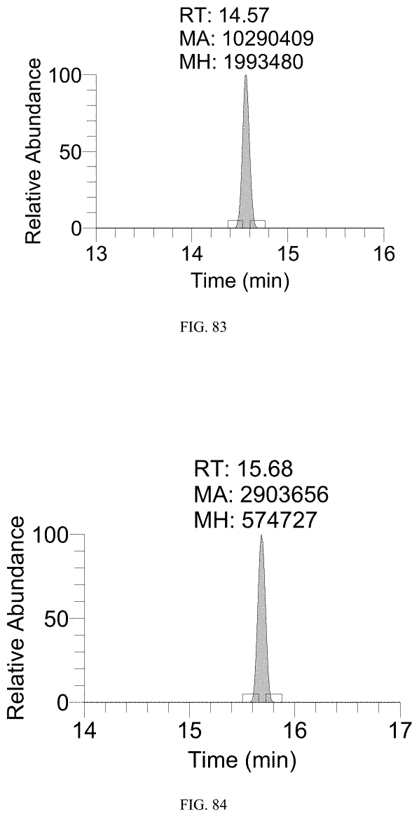

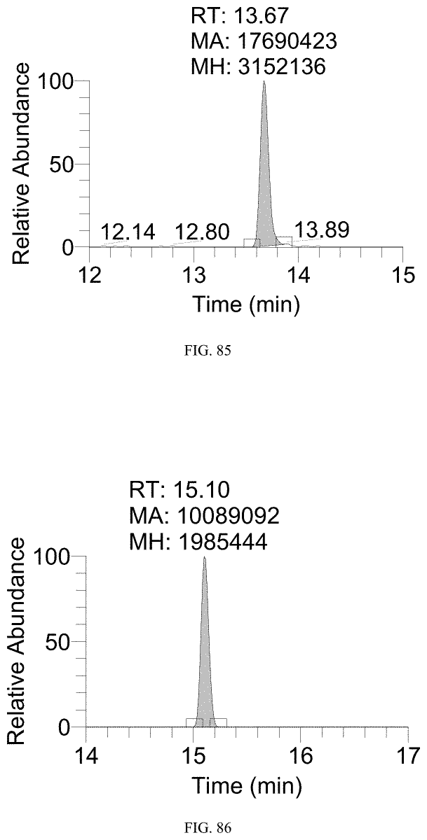

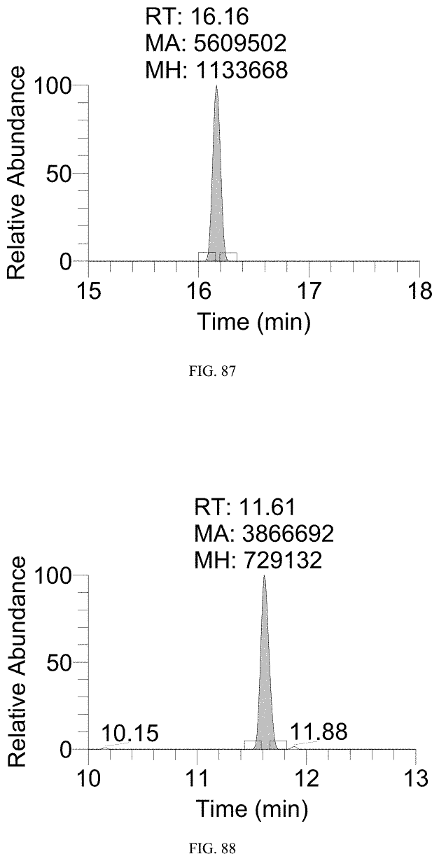

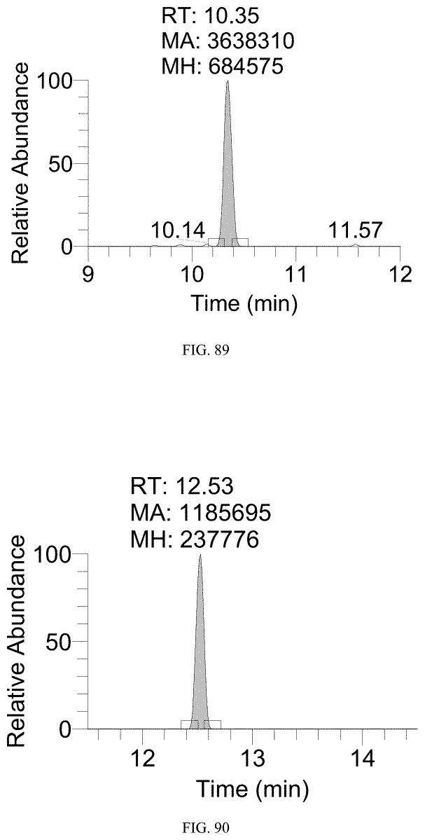

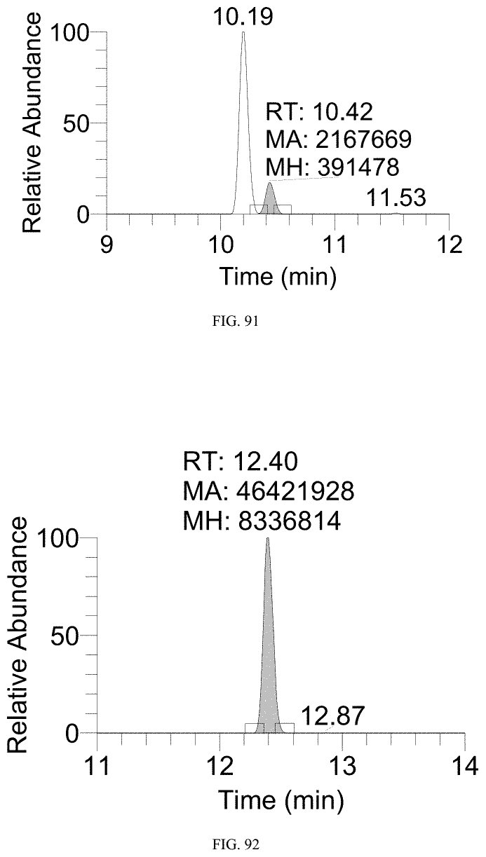

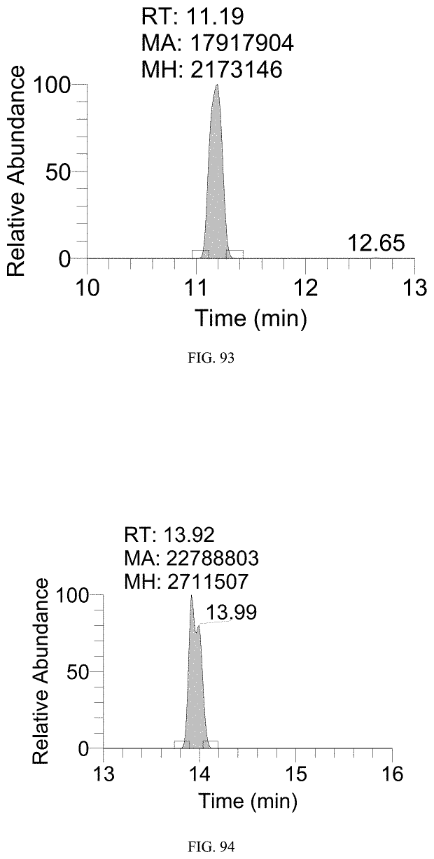

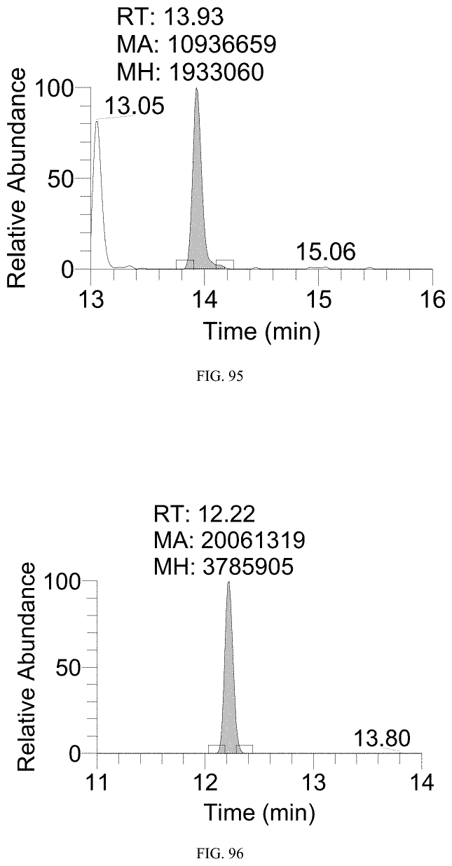

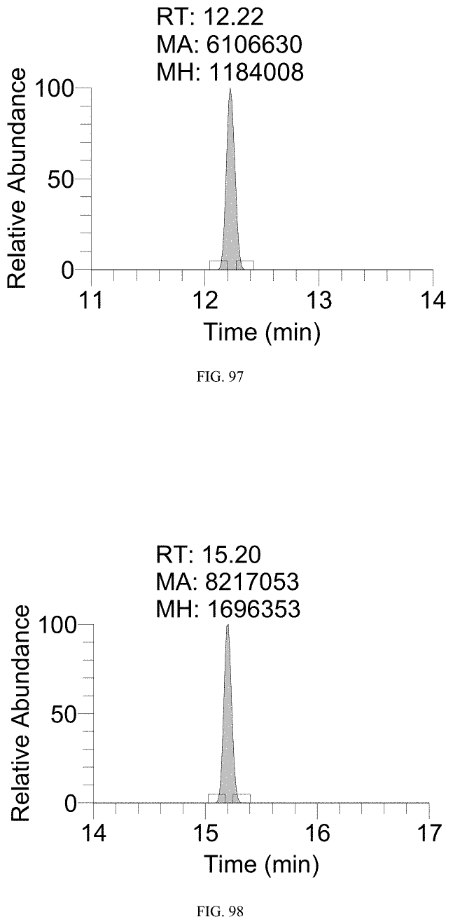

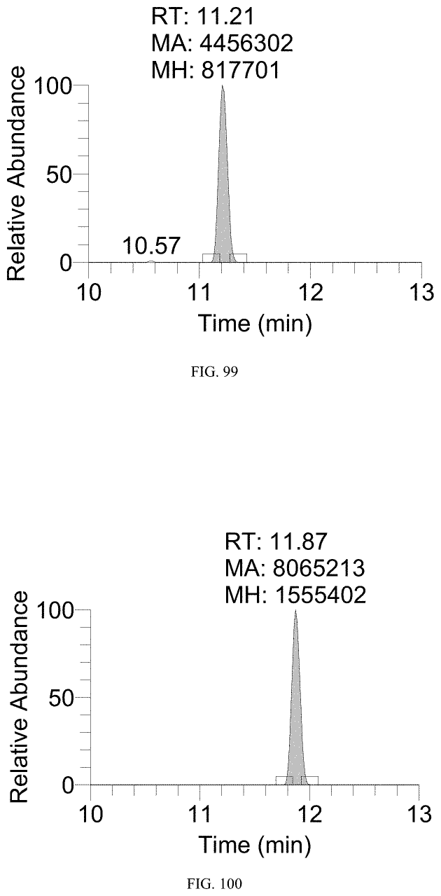

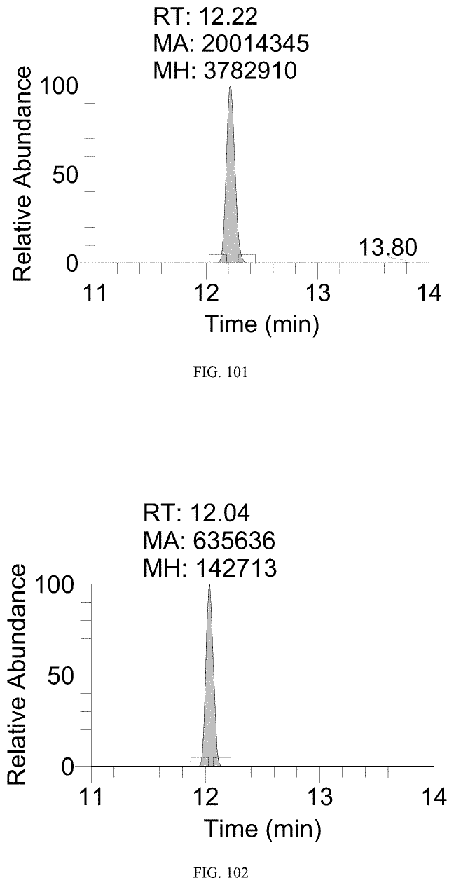

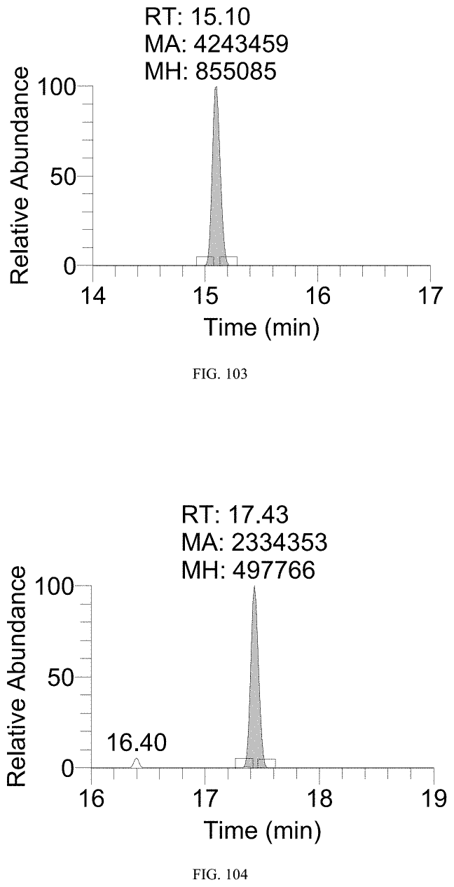

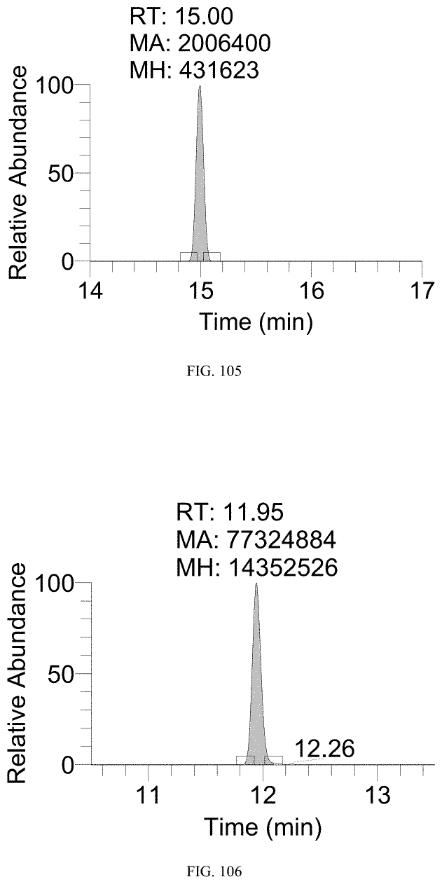

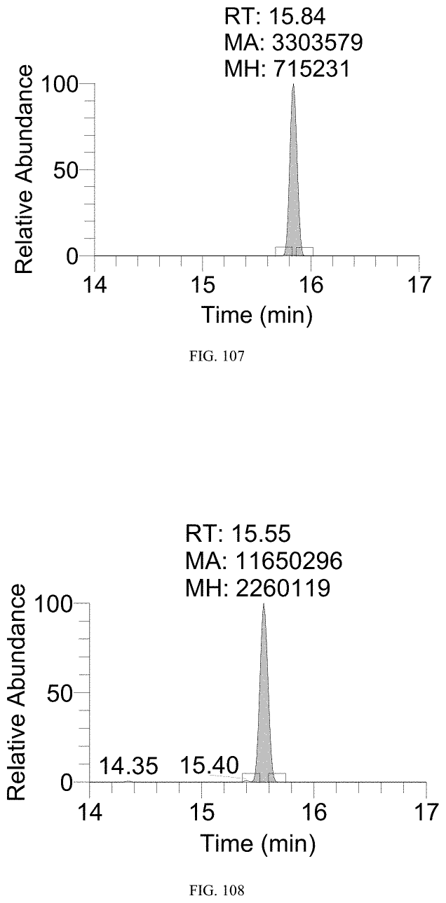

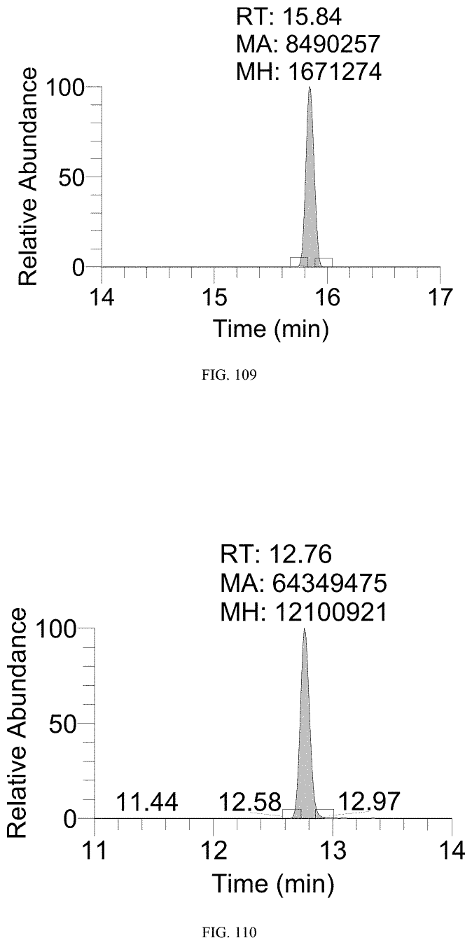

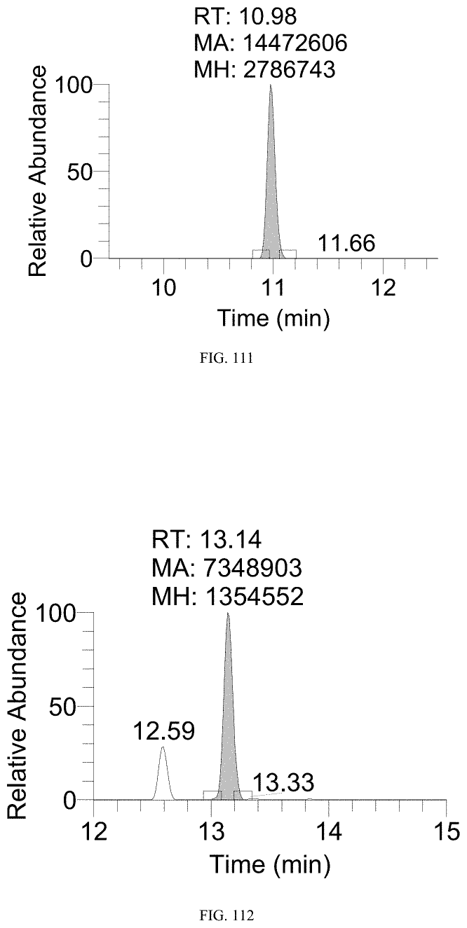

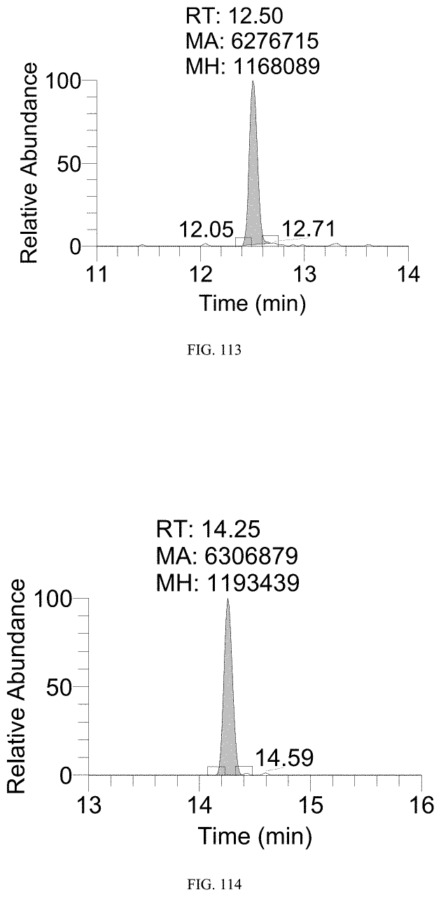

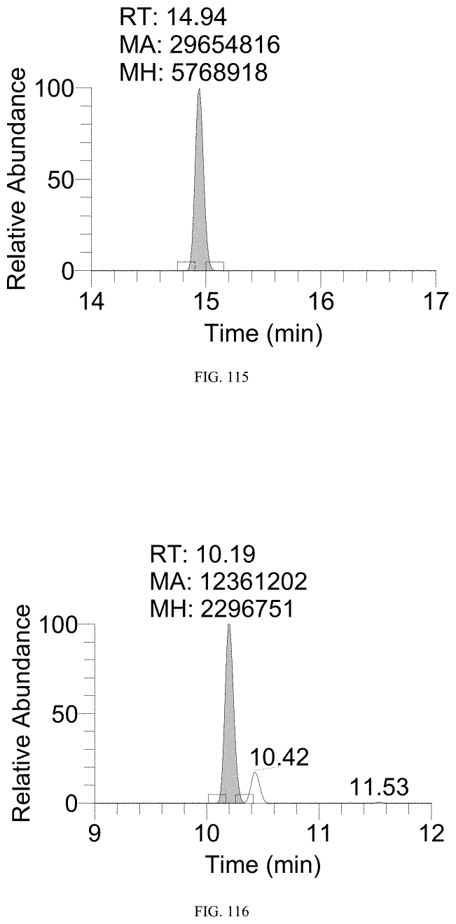

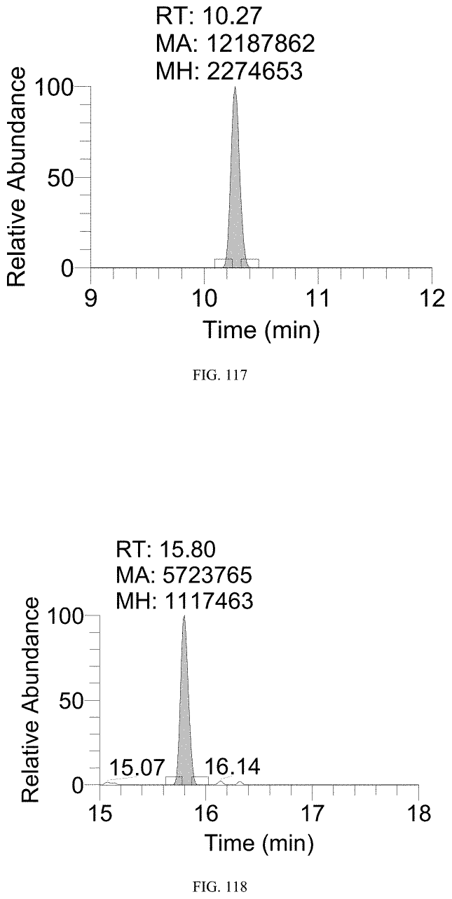

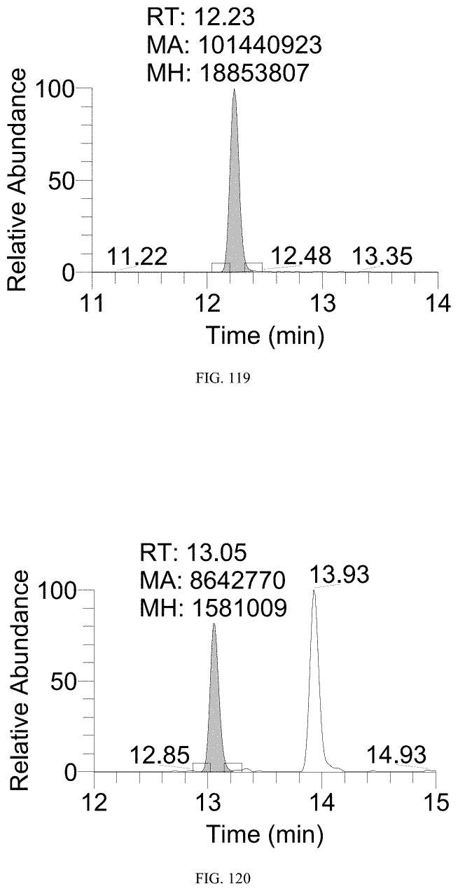

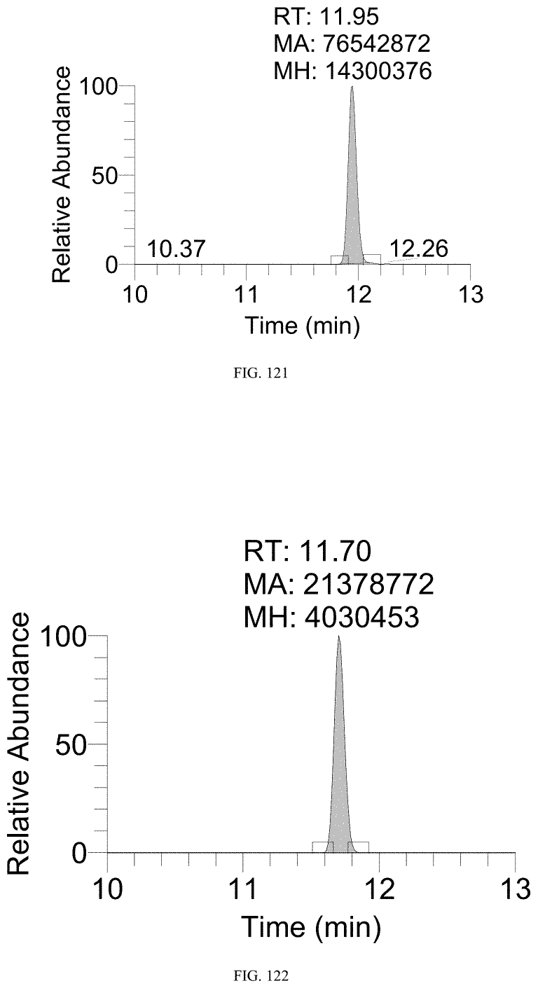

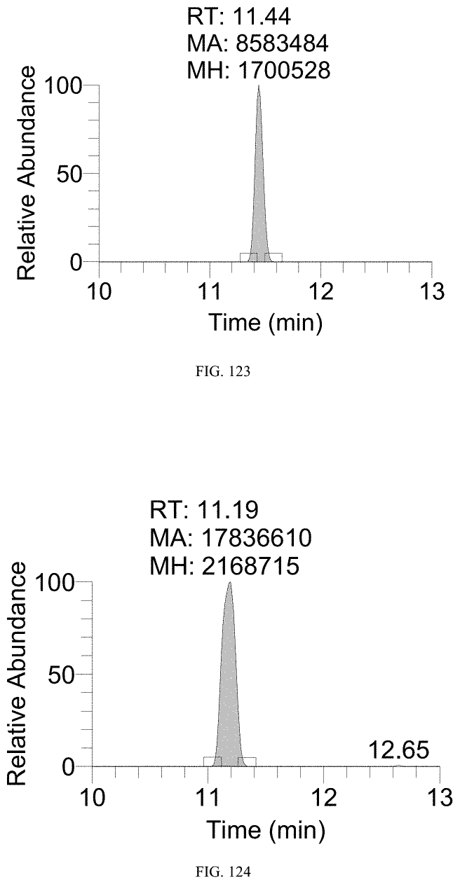

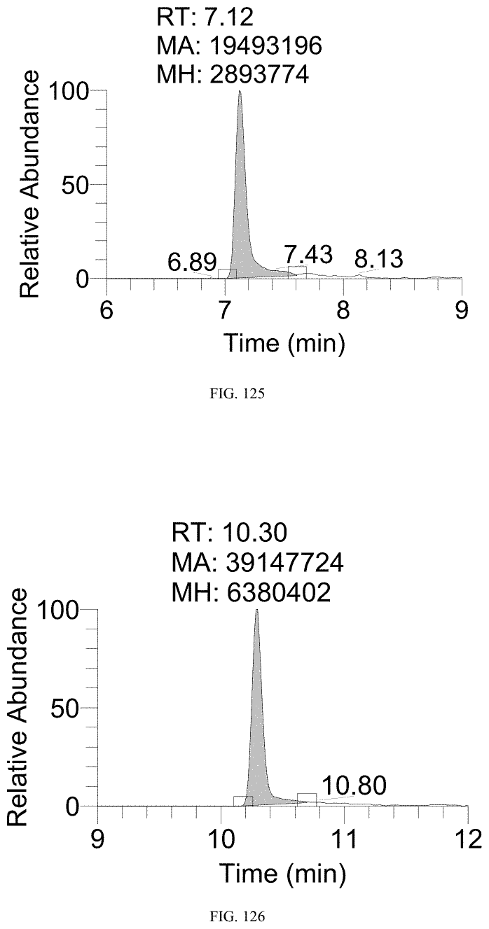

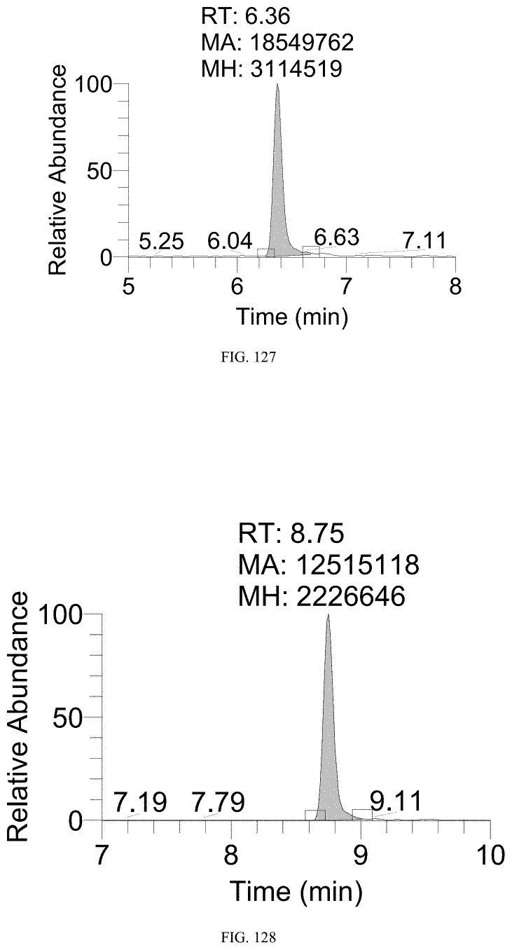

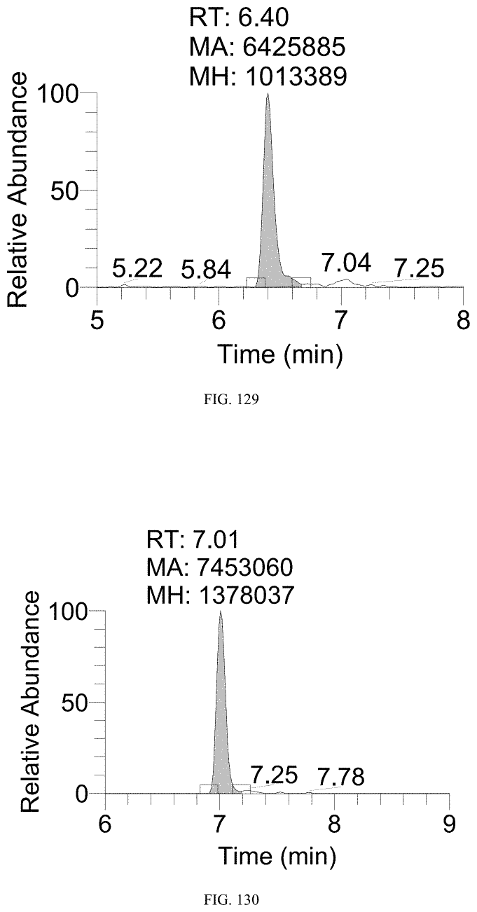

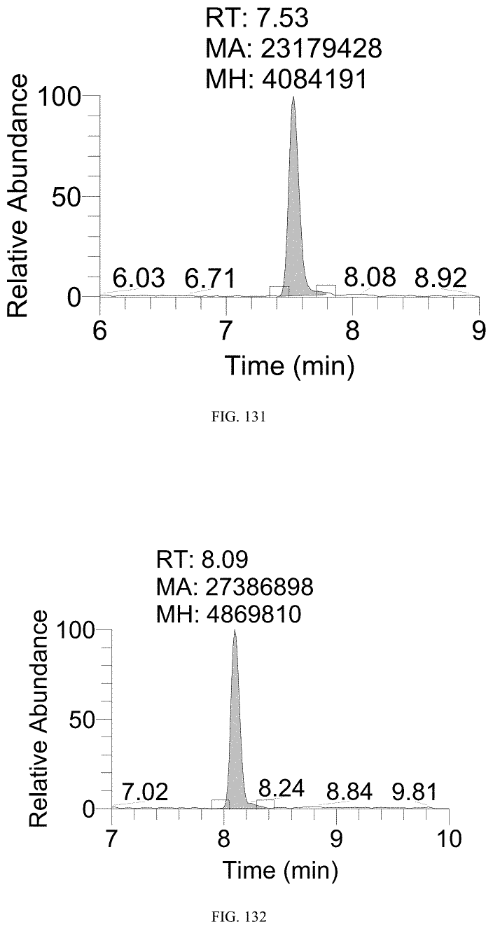

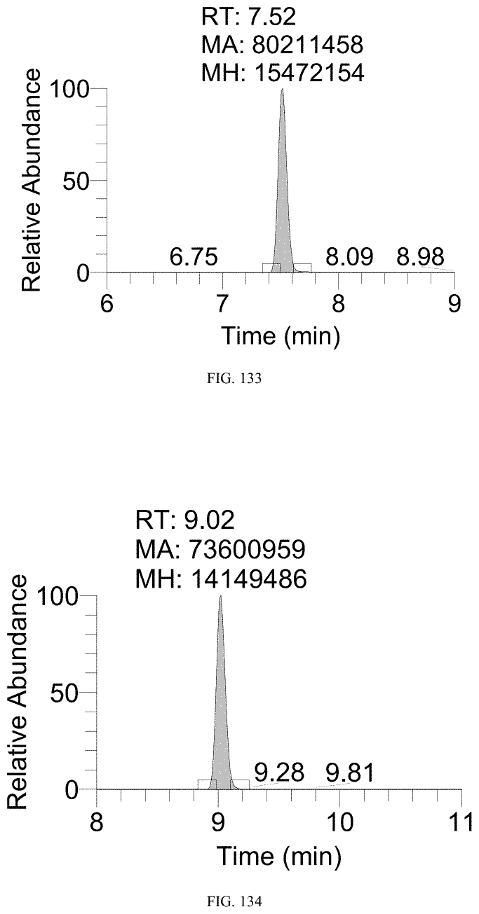

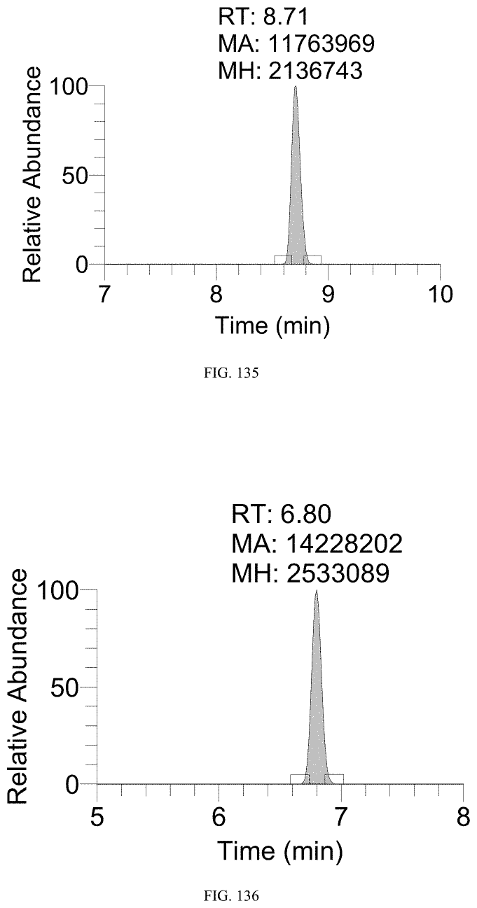

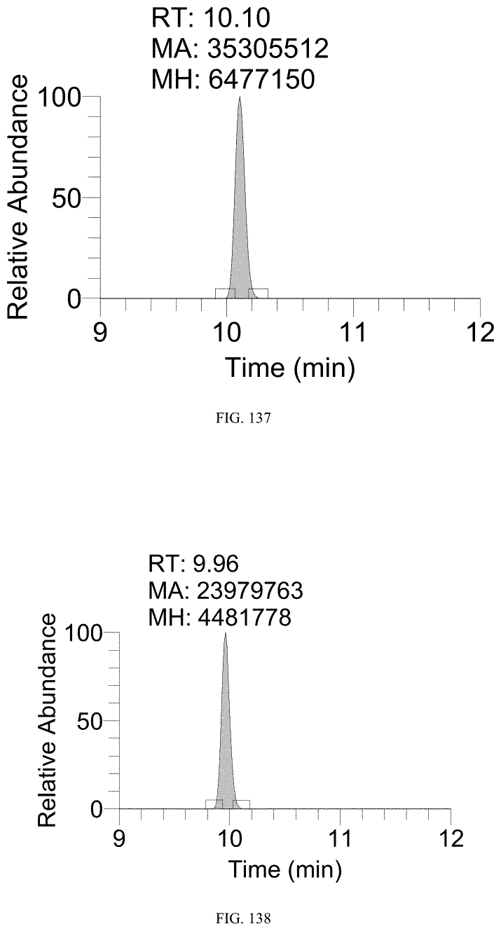

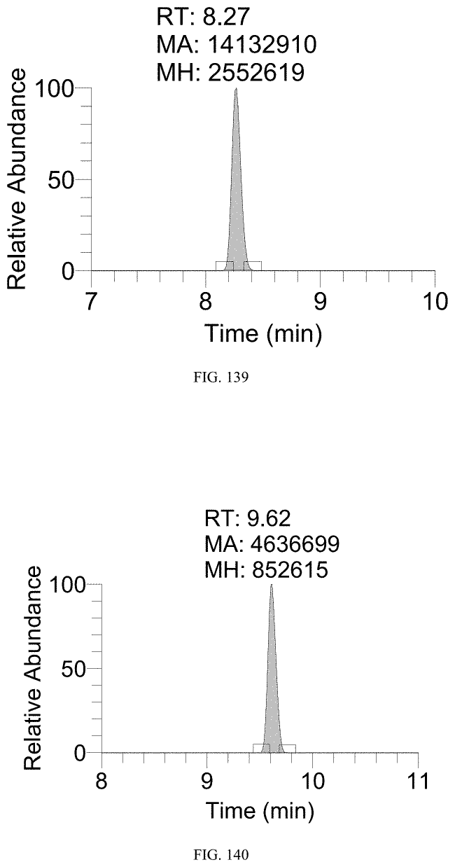

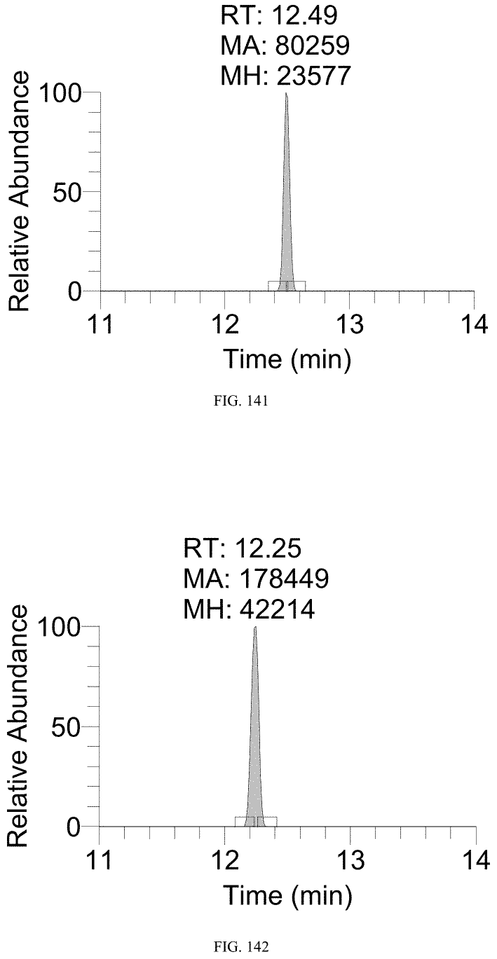

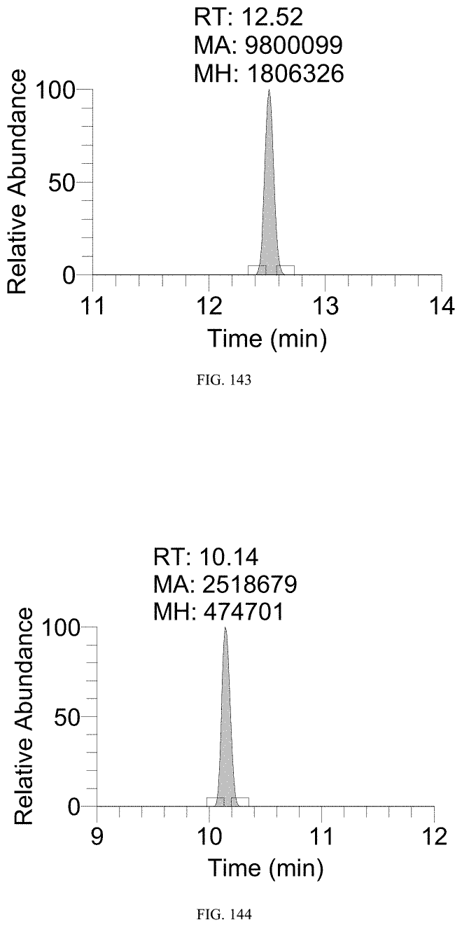

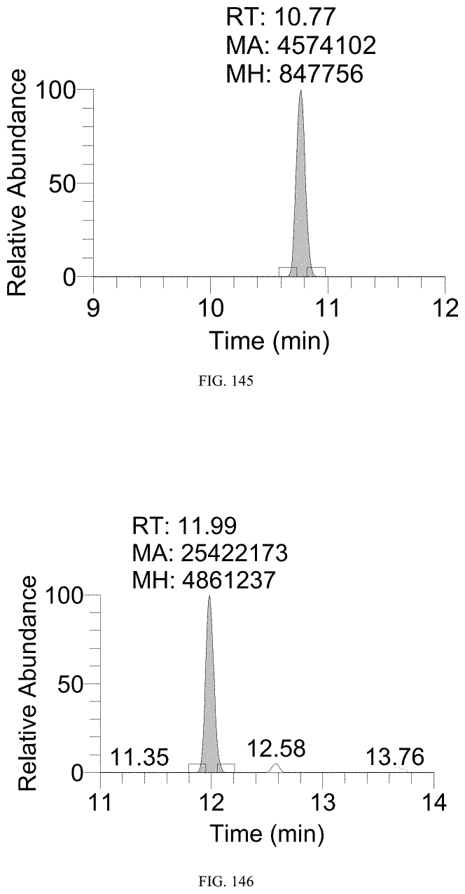

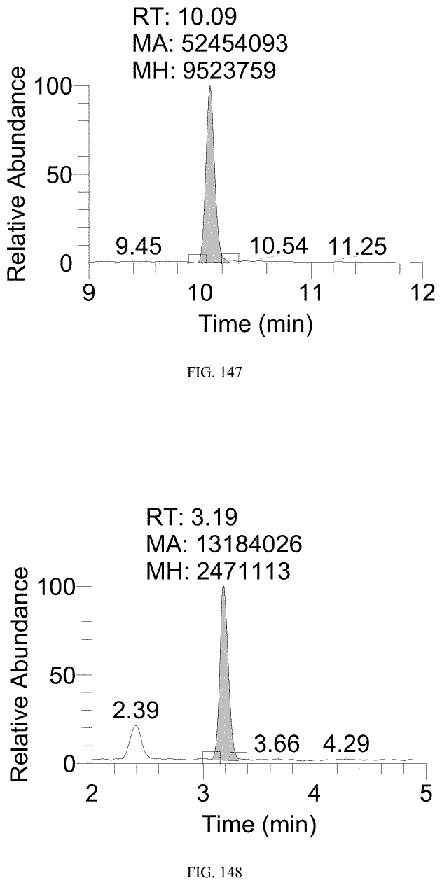

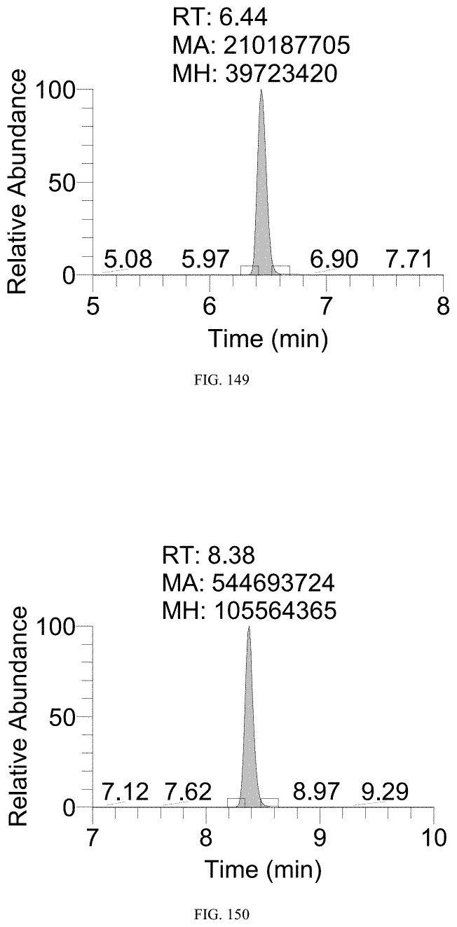

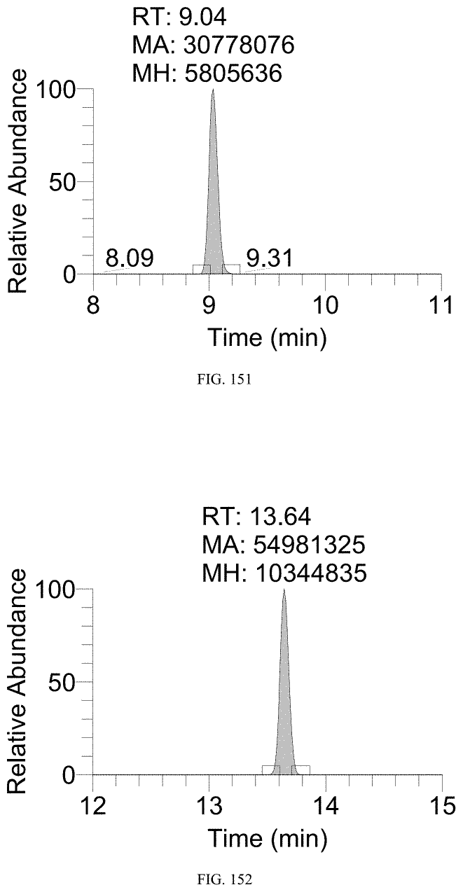

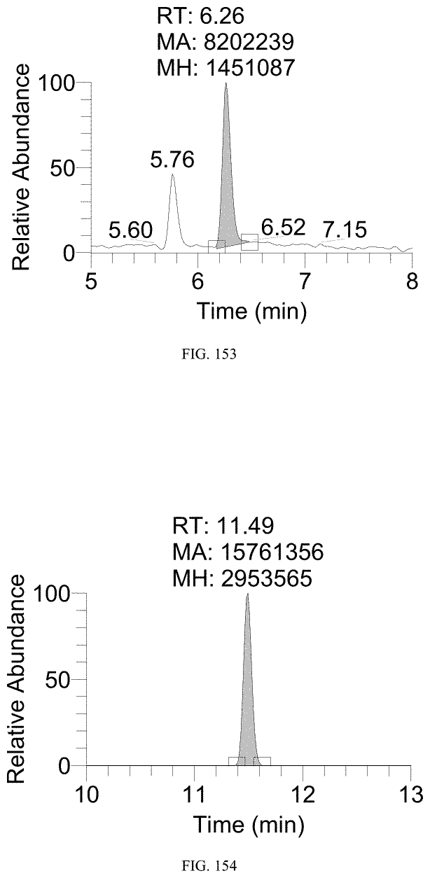

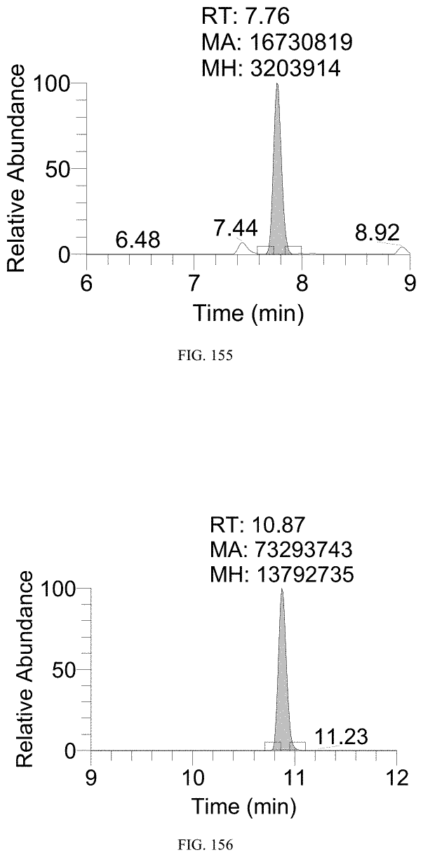

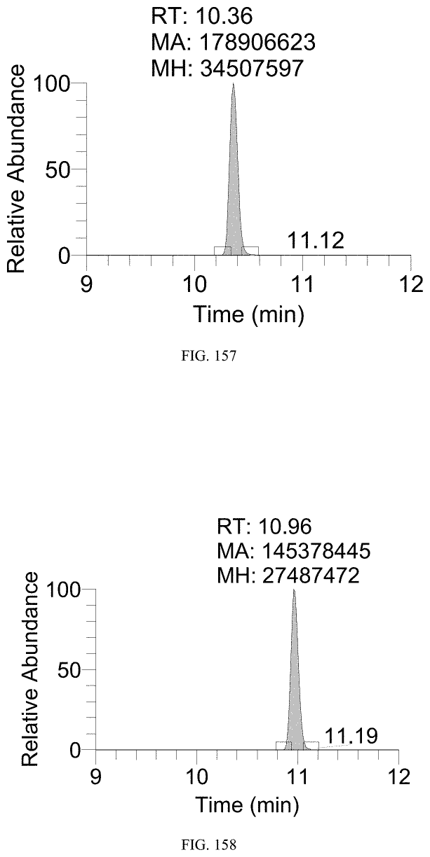

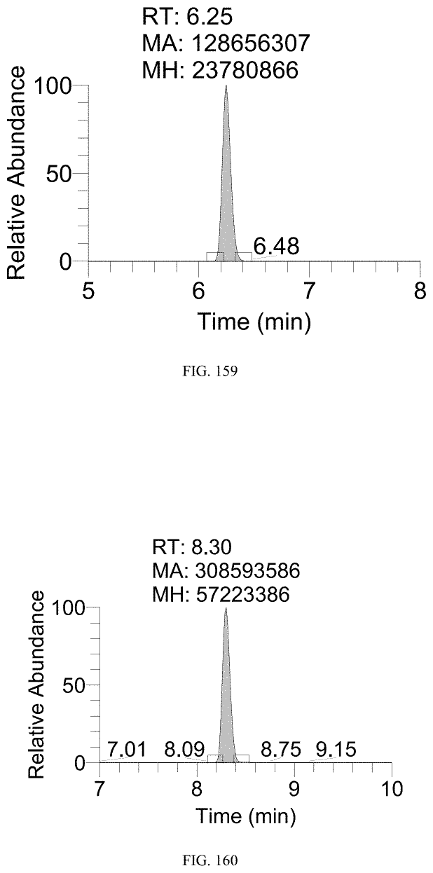

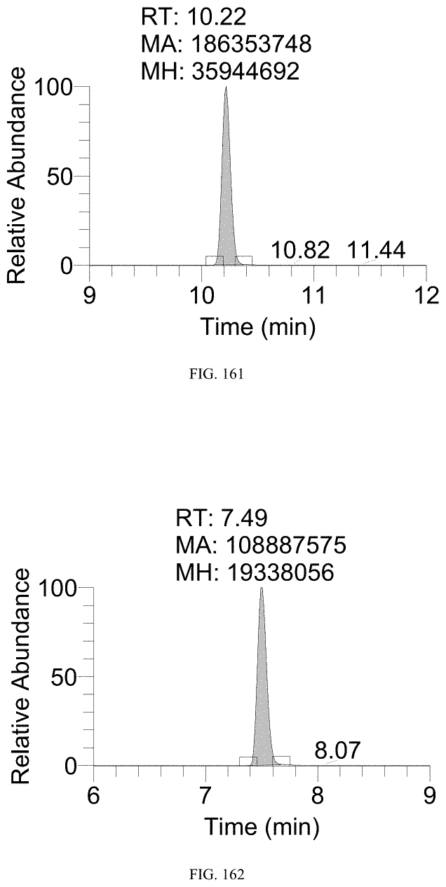

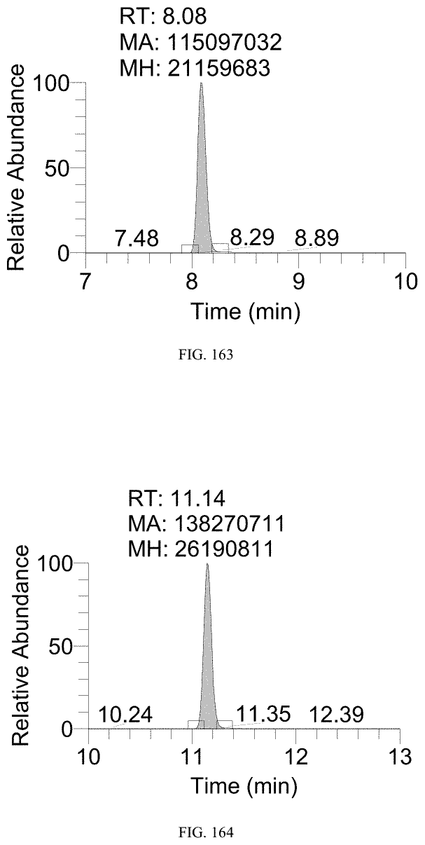

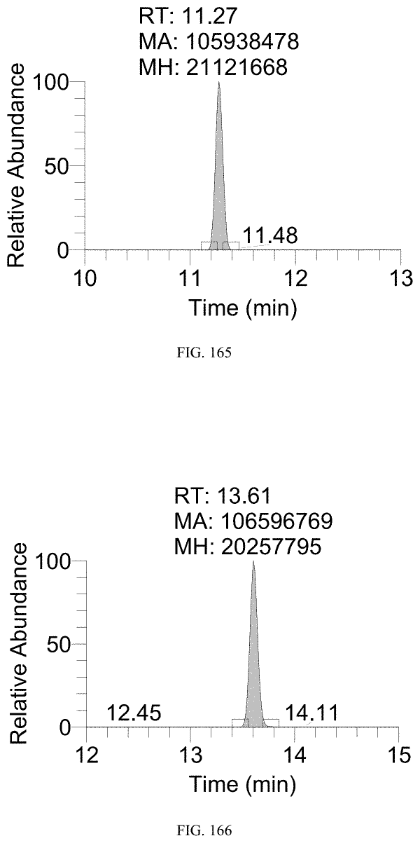

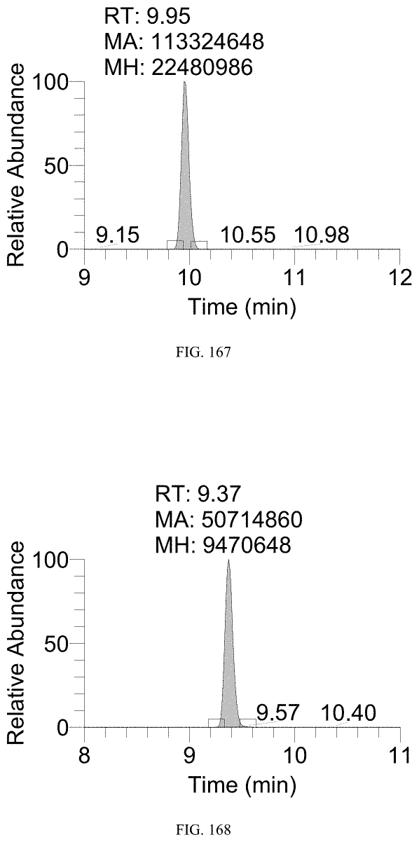

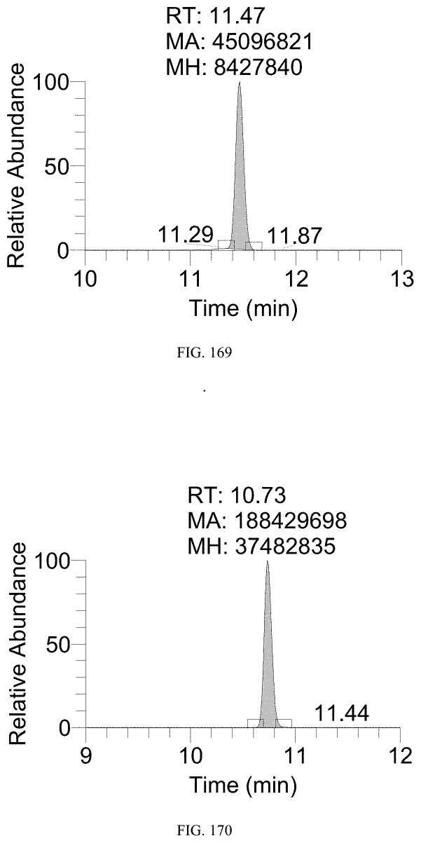

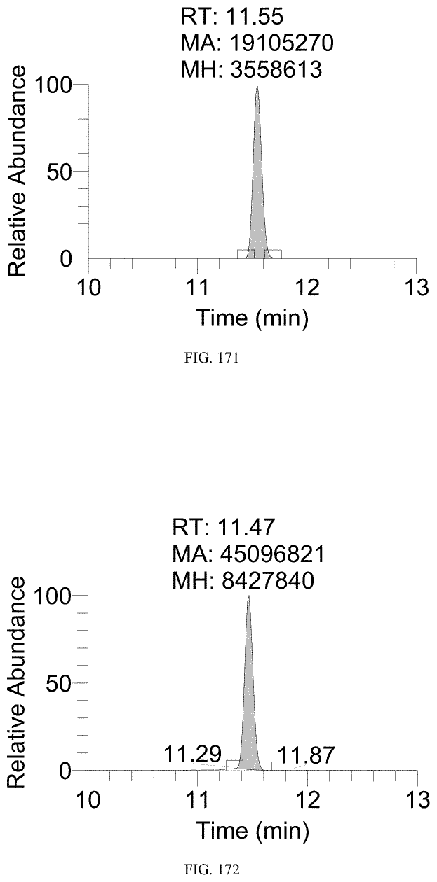

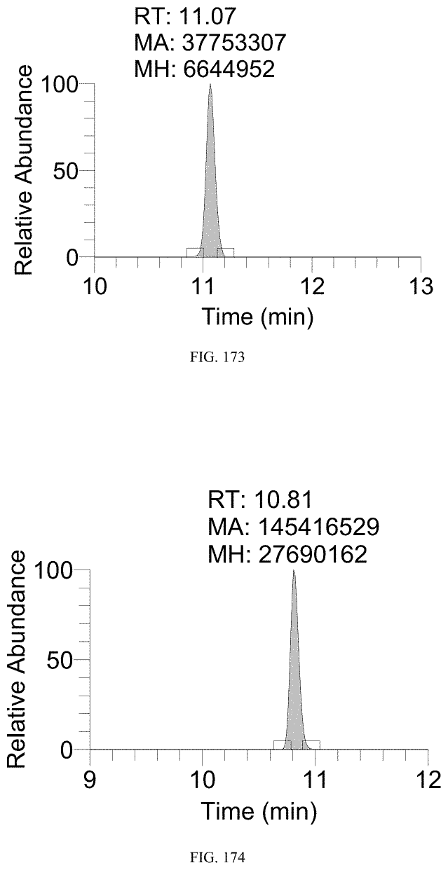

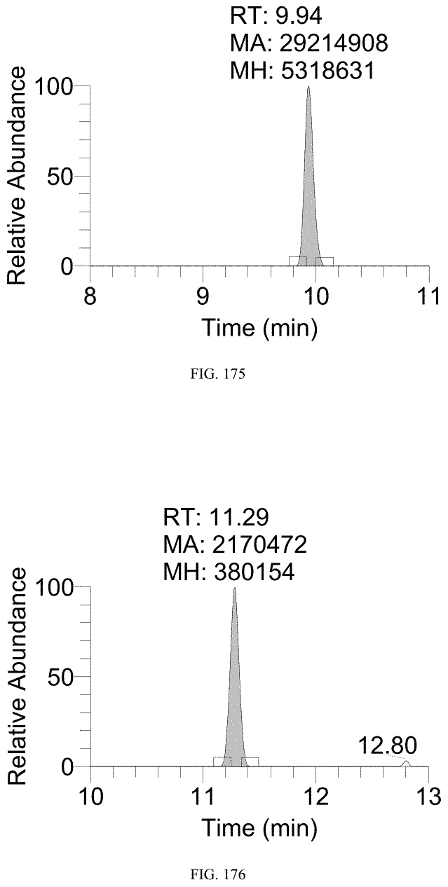

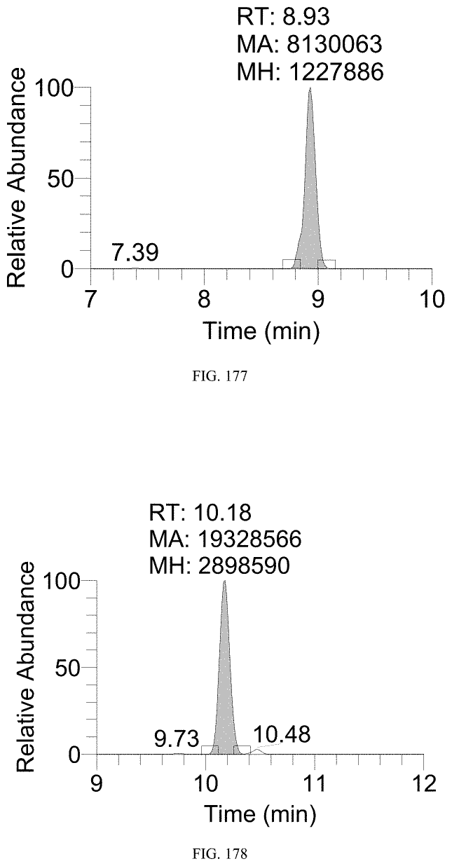

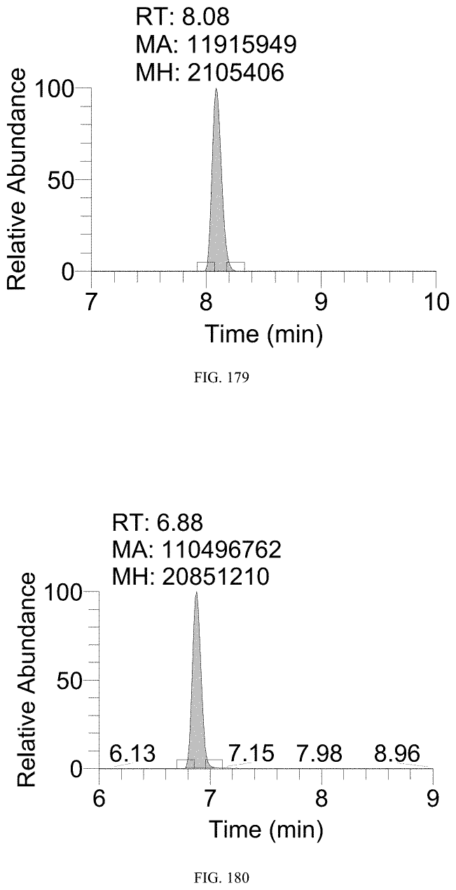

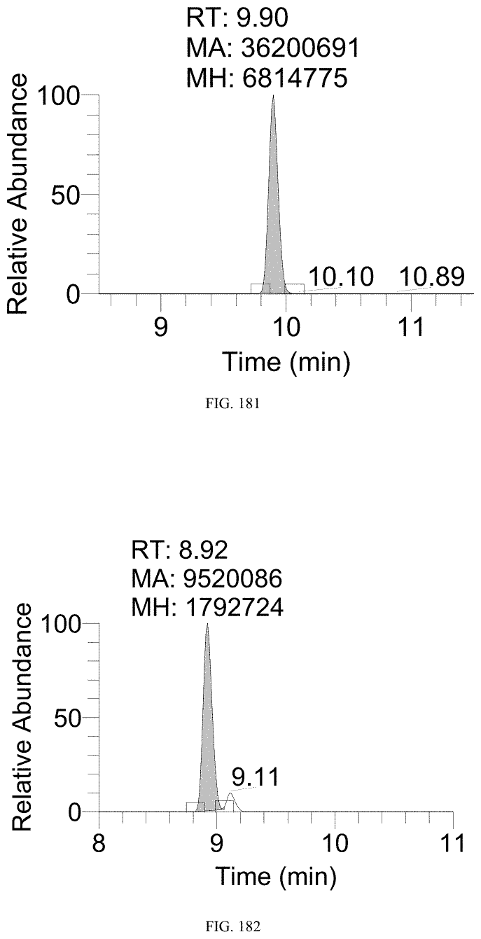

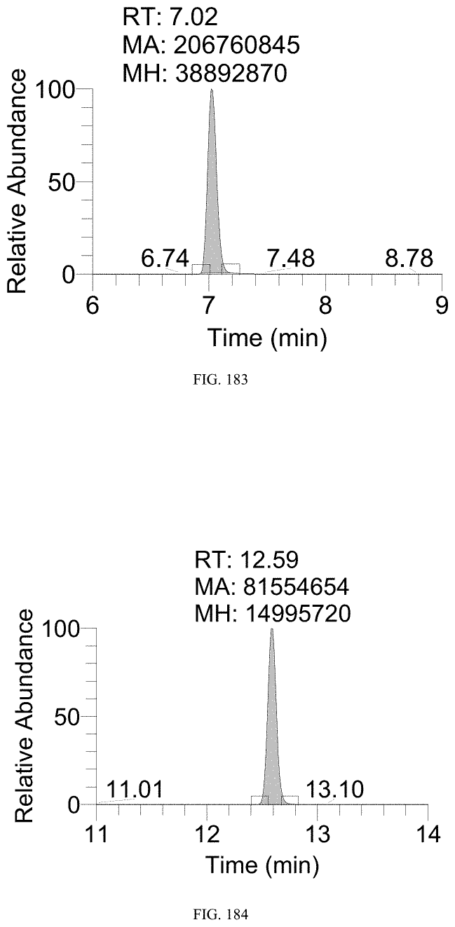

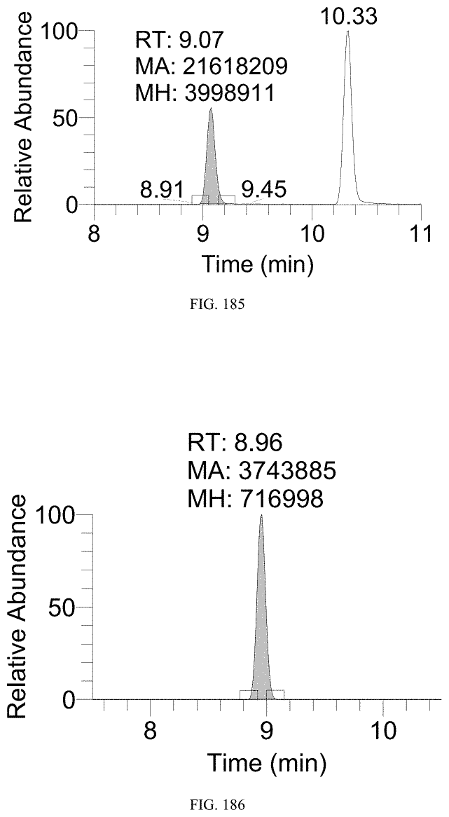

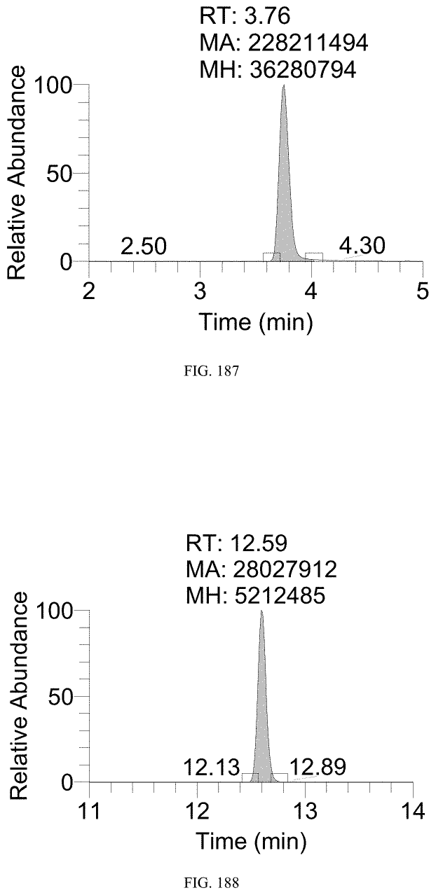

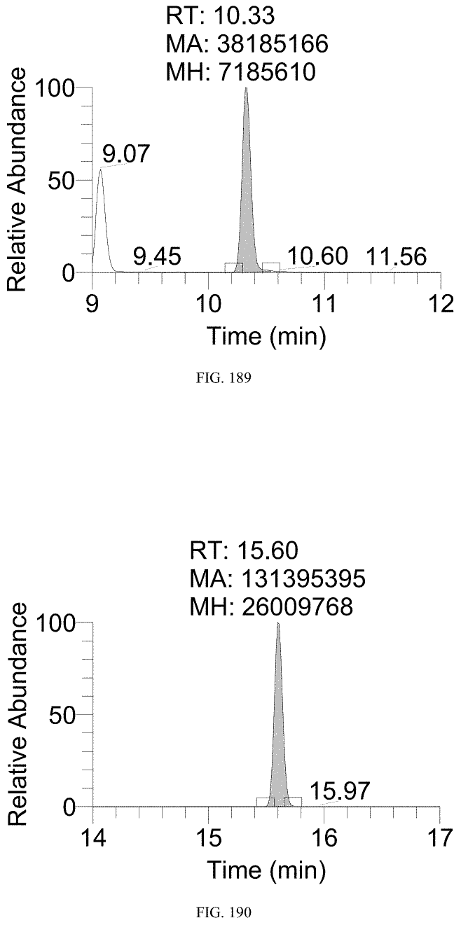

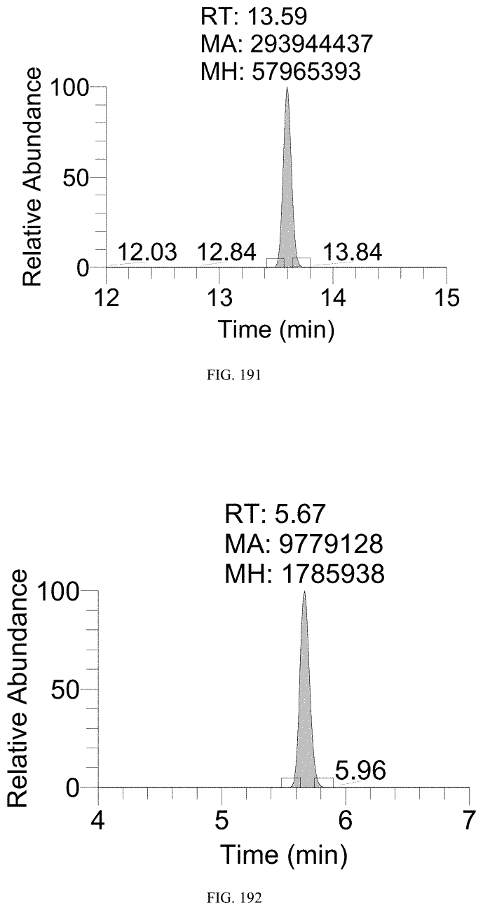

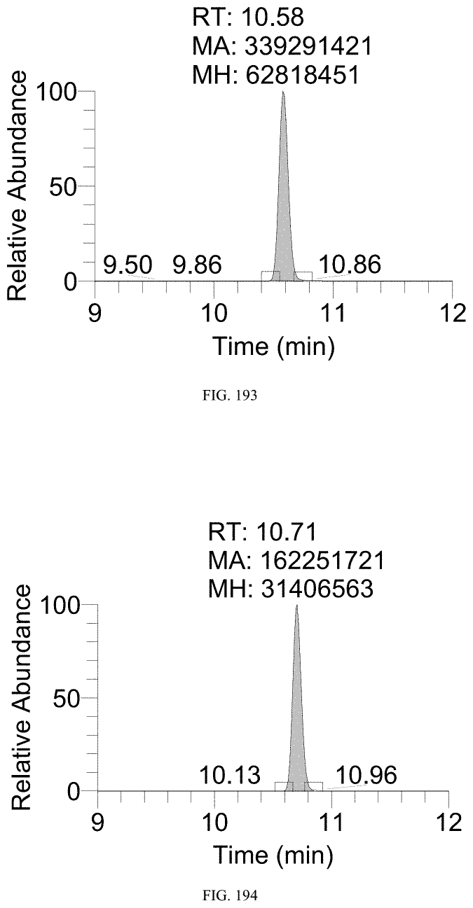

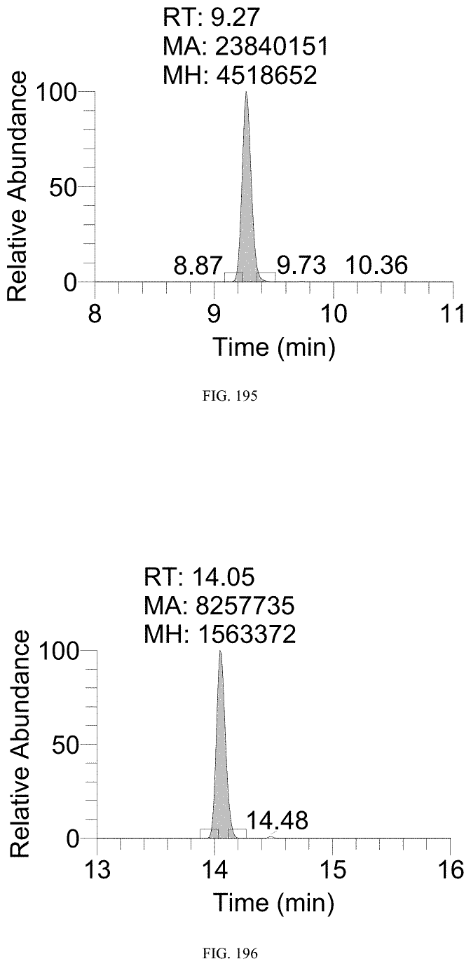

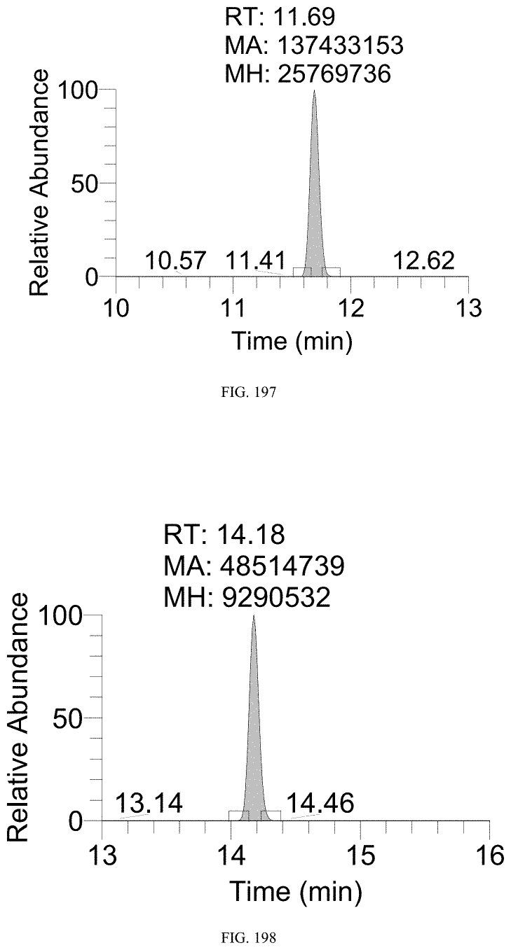

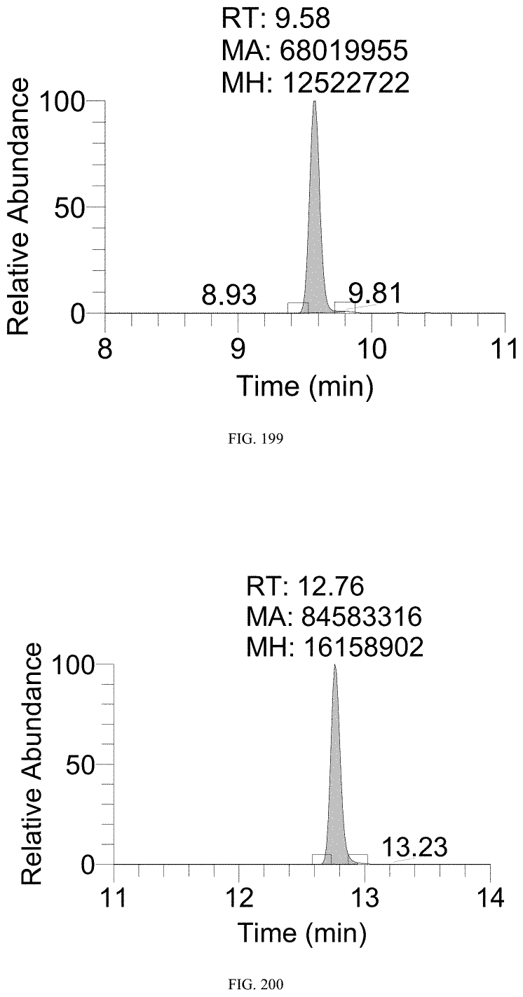

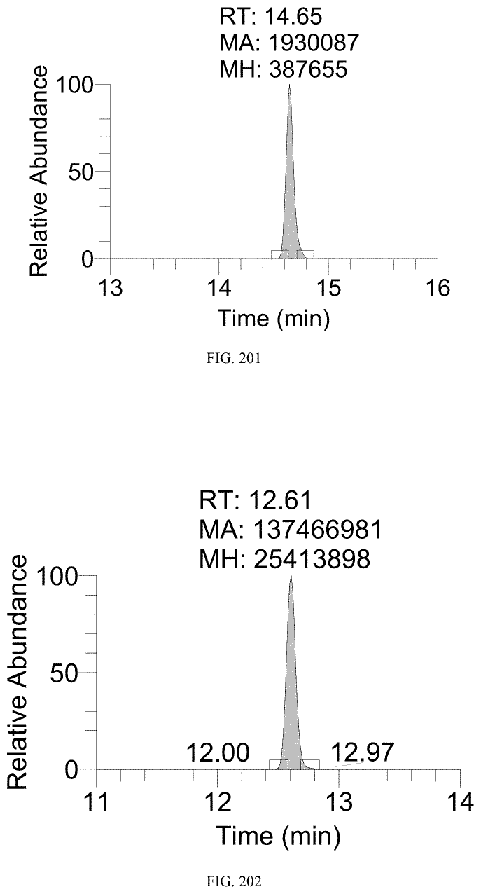

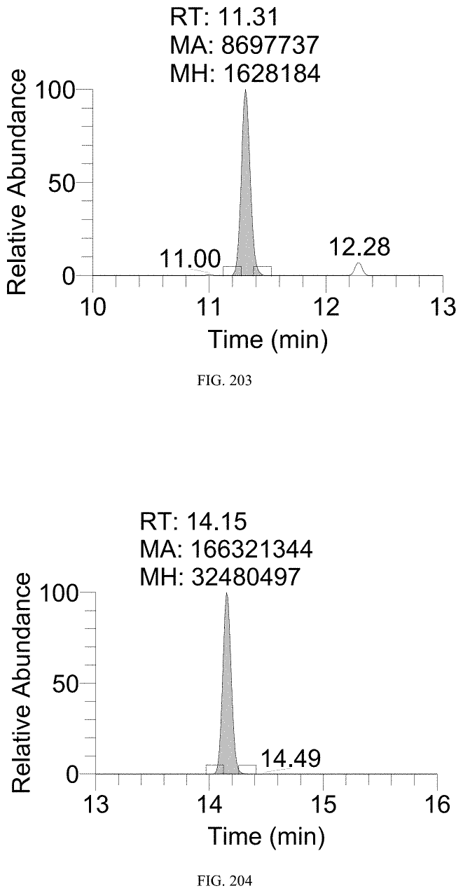

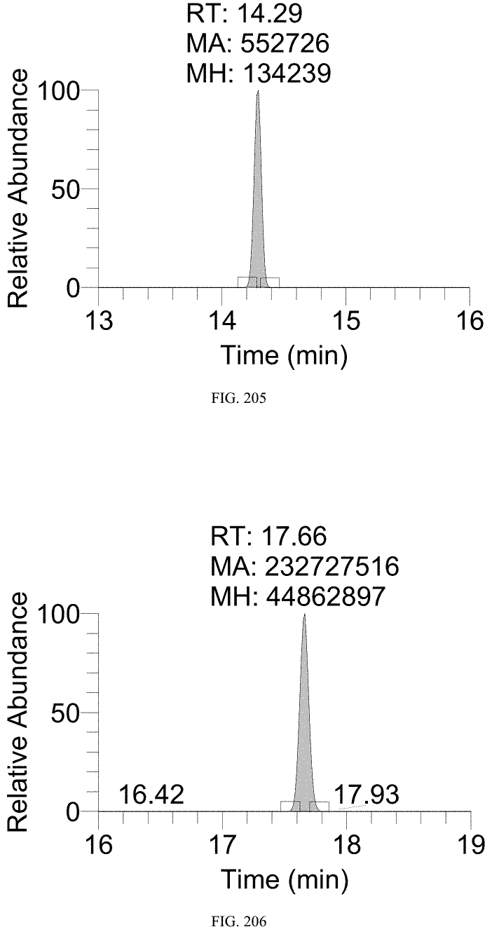

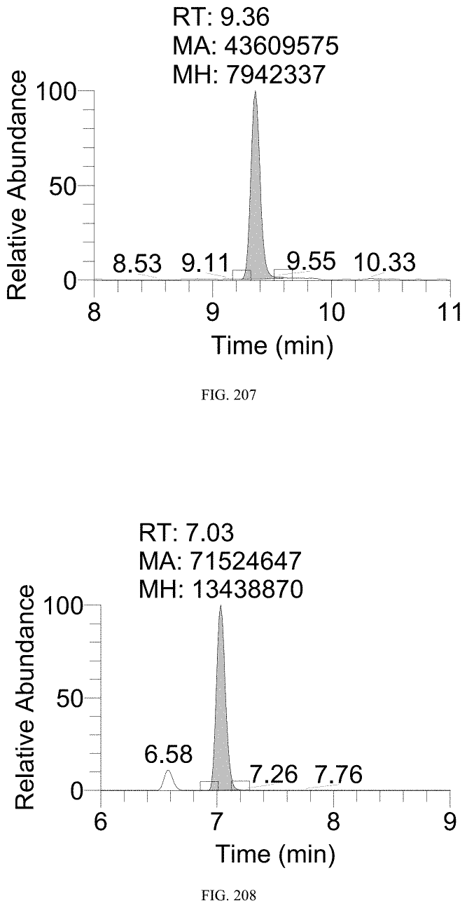

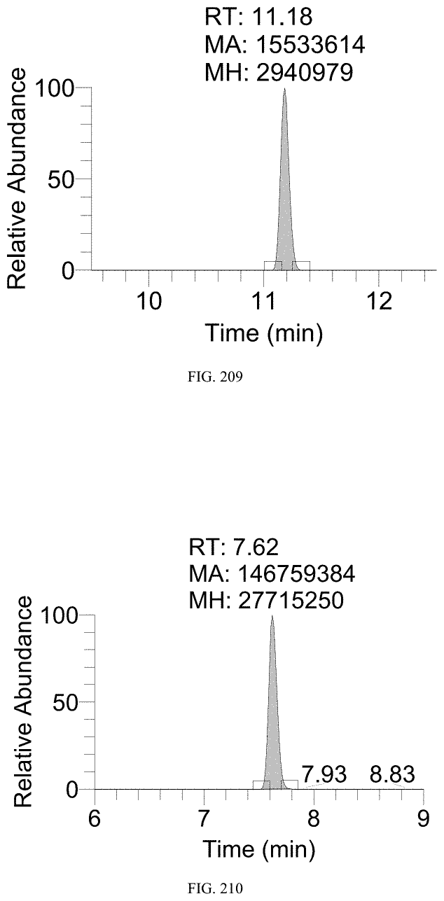

The 16 first mixed standard solutions were diluted such that each reference standard in the corresponding diluted first mixed standard solution thereof had a concentration of 1 μg/mL. Each of the diluted first mixed standard solutions was subjected to full scan and secondary scan in the positive and negative modes using ultra high performance liquid chromatography-quadrupole/electrostatic field orbitrap high resolution mass spectrometer. The obtained scan results were analyzed using Trace Finder software of Q-Exactive plus to obtain information of each reference standard, where the information of each reference standard included retention time, precise molecular weight of parent ion, addition mode, fragment ions, isotope abundance ratio and a fragment ion mass spectrometry generated through the superposition of fragment ion mass spectrometries obtained at collision energies of 20 eV, 40 eV and 60 eV. A mass spectrometry database was established based on the information of each reference standard, which was shown in Table 1. Those isomerides were further identified through the analysis of single reference standard. Enrofloxacin (1 μg/mL) was used as an example, and its parent ion extraction chromatogram, parent ion and fragment ion mass spectrometry were shown in FIGS. 1-3, respectively.

Table 1 Mass spectrometry library information

TABLE 1
Mass spectrometry library information

| | | | | | | | Retention |
|---|---|---|---|---|---|---|---|
| | | Molecular | Addition | Parent | | | time |
| Category | Compound | formula | mode | ion | Fragment 1 | Fragment 2 | (min) |
| Sulfonamides | Sulfamethazine | C₁₂H₁₄N₄O₂S | M + H | 279.09102 | 156.01138 | 124.08694 | 8.44 |
| | Sulformethoxine | C₁₂H₁₄N₄O₄S | M + H | 311.08085 | 156.01138 | 140.04545 | 9.55 |
| | Sulfisoxazole | C₁₁H₁₃N₃O₃S | M + H | 268.07504 | 156.0111 | 108.04462 | 9.83 |
| | Sulfaguanidine | C₇H₁₀N₄O₂S | M + H | 215.05972 | 156.01138 | 108.04439 | 3.43 |
| | Sulfamerazine | C₁₁H₁₂N₄O₂S | M + H | 265.07537 | 190.02809 | 156.01138 | 7.89 |
| | Sulfamethylthiadizaole | C₉H₁₀N₄O₂S₂ | M + H | 271.03179 | 156.01138 | 108.04439 | 8.31 |
| | Sulfamethoxazole | C₁₀H₁₁N₃O₃S | M + H | 254.05939 | 156.01138 | 108.04439 | 9.57 |
| | Sulfamethoxydiazine | C₁₁H₁₂N₄O₃S | M + H | 281.07029 | 108.04465 | 156.01115 | 8.53 |
| | Sulfamethoxypyridazine | C₁₁H₁₂N₄O₃S | M + H | 281.07029 | 215.09274 | 156.01138 | 8.41 |
| | Sulfamonomethoxine | C₁₁H₁₂N₄O₃S | M + H | 281.07029 | 215.09274 | 156.01138 | 8.9 |
| | Sulfamethazole | C₁₁H₁₃N₃O₃S | M + H | 268.07504 | 156.0111 | 108.04462 | 8.03 |
| | Sulfapyridinee | C₁₁H₁₁N₃O₂S | M + H | 250.06447 | 184.08692 | 156.01138 | 7.55 |
| | Sulfaquinoxaline | C₁₄H₁₂N₄O₂S | M + H | 301.07537 | 156.01138 | 108.04439 | 10.39 |
| | Sulfathiazole | C₉H₉N₃O₂S₂ | M + H | 256.02089 | 156.01138 | 108.04439 | 7.28 |
| | Sulfisomidine | C₁₂H₁₄N₄O₂S | M + H | 279.09102 | 124.08692 | 108.04439 | 6.57 |
| | Trimethoprim | C₁₄H₁₈N₄O₃ | M + H | 291.14517 | 275.11387 | 261.09822 | 7.28 |
| | Diaveridine | C₁₃H₁₆N₄O₂ | M + H | 261.1346 | 245.1033 | 123.06652 | 7 |
| | Phthalylsulfathiazole | C₁₇H₁₃N₃O₅S₂ | M + H | 404.03694 | 149.02313 | 256.02032 | 9.07 |
| | Sulfabenzamide | C₁₃H₁₂N₂O₃S | M + H | 277.06414 | 156.01138 | 108.04439 | 10.32 |
| | Sulfacetamide | C₈H₁₀N₂O₃S | M + H | 215.04849 | 173.03792 | 156.01138 | 6.73 |
| | Sulfachlorpyridazine | C₁₀H₉ClN₄O₂S | M + H | 285.02075 | 156.01138 | 108.04439 | 9.25 |
| | Sulfachloropyrazine | C₁₀H₉ClN₄O₂S | M + H | 285.02075 | 130.0166 | 156.01111 | 10.35 |
| | Sulfadiazine | C₁₀H₁₀N₄O₂S | M + H | 251.05972 | 156.01138 | 108.04439 | 7.13 |
| | Sulfadimethoxine | C₁₂H₁₄N₄O₄S | M + H | 311.08085 | 156.07675 | 108.04439 | 10.43 |
| Fluoroquinolones | Lomefloxacin | C₁₇H₁₉F₂N₃O₃ | M + H | 352.14672 | 308.1569 | 265.1147 | 7.58 |
| | Marbofloxacin | C₁₇H₁₉FN₄O₄ | M + H | 363.14631 | 320.10411 | 72.08078 | 7.17 |
| | Nadifloxacin | C₁₉H₂₁FN₂O₄ | M + H | 361.15581 | 343.14389 | 283.08725 | 10.76 |
| | Nalidixic acid | C₁₂H₁₂N₂O₃ | M + H | 233.09207 | 205.06077 | 187.0502 | 11.43 |
| | Norfloxacin | C₁₆H₁₈FN₃O₃ | M + H | 320.1405 | 276.15067 | 233.10847 | 7.24 |
| | Ofloxacin | C₁₈H₂₀FN₃O₄ | M + H | 362.15106 | 318.16123 | 261.10338 | 7.31 |
| | Orbifloxacin | C₁₉H₂₀F₃N₃O₃ | M + H | 396.15295 | 352.16312 | 295.10527 | 7.89 |
| | Oxolinic acid | C₁₃H₁₁NO₅ | M + H | 262.071 | 244.06043 | 234.0397 | 9.95 |
| | Pipemidic acid | C₁₄H₁₇N₅O₃ | M + H | 304.14042 | 290.16632 | 233.10847 | 6.75 |
| | Pefloxacin | C₁₇H₂₀FN₃O₃ | M + H | 334.15615 | 217.10839 | 189.0779 | 7.35 |
| | Cinoxacin | C₁₂H₁₀N₂O₅ | M + H | 263.06625 | 235.07133 | 217.06077 | 9.47 |
| | Sarafloxacin | C₂₀H₁₇F₂N₃O₃ | M + H | 386.13107 | 342.14125 | 299.09905 | 8.16 |
| | Sparfloxacin | C₁₉H₂₂F₂N₄O₃ | M + H | 393.17327 | 349.18344 | 292.12559 | 8.22 |
| | Ciprofloxacin | C₁₇H₁₈FN₃O₃ | M + H | 332.1405 | 288.15067 | 245.10847 | 7.37 |
| | Danofloxacin | C₁₉H₂₀FN₃O₃ | M + H | 358.15615 | 314.16632 | 283.12412 | 7.54 |
| | Difloxacin | C₂₁H₁₉F₂N₃O₃ | M + H | 400.14672 | 356.15689 | 299.09905 | 8.27 |
| | Enoxacin | C₁₅H₁₇FN₄O₃ | M + H | 321.13575 | 277.14592 | 257.13969 | 7.13 |
| | Enrofloxacin | C₁₉H₂₂FN₃O₃ | M + H | 360.1718 | 316.18197 | 245.10847 | 7.73 |
| | Fleroxacin | C₁₇H₁₈F₃N₃O₃ | M + H | 370.1373 | 326.14747 | 269.08962 | 7.36 |
| | Flumequine | C₁₄H₁₂FNO₃ | M + H | 262.0874 | 244.07683 | 220.04045 | 11.59 |
| | Gatifloxacin | C₁₉H₂₂FN₃O₄ | M + H | 376.16671 | 332.17688 | 289.13468 | 7.97 |
| Stimulants | Cyproheptadine | C₂₁H₂₁N | M + H | 288.17468 | 191.08553 | 96.08078 | 10.67 |
| | Fenoterol | C₁₇H₂₁NO₄ | M + H | 304.15433 | 286.14377 | 135.08044 | 6.57 |
| | Labetalol | C₁₉H₂₄N₂O₃ | M + H | 329.18597 | 311.1754 | 294.14886 | 8.97 |
| | Penbutolol | C₁₈H₂₉NO₂ | M + H | 292.22711 | 236.16451 | 201.12739 | 11.08 |
| | Phenylethanolamine A | C₁₉H₂₄N₂O₄ | M + H | 345.18088 | 327.17032 | 150.09134 | 10.03 |
| | Ractopamine | C₁₈H₂₃NO₃ | M + H | 302.17507 | 284.16451 | 164.10699 | 7.56 |
| | Salbutamol | C₁₃H₂₁NO₃ | M + H | 240.15942 | 148.07569 | 166.08625 | 5.75 |
| | Salmeterol | C₂₅H₃₇NO₄ | M + H | 416.27954 | 398.26897 | 380.2584 | 11.03 |
| | Terbutaline | C₁₂H₁₉NO₃ | M + H | 226.14377 | 170.08117 | 152.07061 | 5.71 |
| | Mabuterol | C₁₃H₁₈ClF₃N₂O | M + H | 311.11325 | 237.03951 | 217.03343 | 9 |
| | Tulobuterol | C₁₂H₁₈ClNO | M + H | 228.11497 | 172.05237 | 154.0418 | 8.25 |
| | Bambuterol | C₁₈H₂₉N₃O₅ | M + H | 368.218 | 312.1554 | 294.14483 | 8.65 |
| | Brombuterol | C₁₂H₁₈Br₂N₂O | M + H | 364.98586 | 290.9127 | 211.99436 | 8.71 |
| | Cimaterol | C₁₂H₁₇N₃O | M + H | 220.14444 | 202.13387 | 160.08692 | 5.93 |
| | Cimbuterol | C₁₃H₁₉N₃O | M + H | 234.16009 | 160.08656 | 143.06007 | 6.61 |
| | Clenbuterol | C₁₂H₁₈Cl₂N₂O | M + H | 277.0869 | 259.07633 | 203.01373 | 8.31 |
| | Clenproperol | C₁₁H₁₆Cl₂N₂O | M + H | 263.07125 | 245.06068 | 203.01373 | 7.85 |
| | Clonidine | C₉H₉Cl₂N₃ | M + H | 230.02463 | 212.99808 | / | 6.77 |
| Hormones | Chlormadinone acetate | C₂₃H₂₉ClO₄ | M + H | 405.18271 | 309.1849 | 267.1743 | 15.67 |
| | 17a-methyltestosterone | C₂₀H₃₀O₂ | M + H | 303.23186 | 285.22129 | 267.21073 | 13.63 |
| | Clobetasol propionate | C₂₅H₃₂ClFO₅ | M + H | 467.19951 | 263.143 | 147.0804 | 15.08 |
| | Clobetasone butyrate | C₂₆H₃₂ClFO₅ | M + H | 479.19951 | 279.138 | 343.1459 | 16.13 |
| | Corticosterone | C₂₁H₃₀O₄ | M + H | 347.22169 | 329.21112 | 311.20056 | 11.61 |
| | Hydrocortisone | C₂₁H₃₀O₅ | M + H | 363.2166 | 327.19547 | 309.18491 | 10.34 |
| | Cortisone acetate | C₂₃H₃₀O₆ | M + H | 403.21152 | 343.19038 | 163.11174 | 12.53 |
| | Cortisone | C₂₁H₂₈O₅ | M + H | 361.20095 | 163.1117 | 121.0648 | 10.45 |
| | Deflazacort | C₂₅H31NO₆ | M + H | 442.22241 | 142.0499 | 312.1958 | 12.39 |
| | Dexamethasone | C₂₂H₂₉FO₅ | M + H | 393.20718 | 355.19039 | 337.17982 | 11.21 |
| | Diflorasone diacetate | C₂₆H₃₂F₂O₇ | M + H | 495.21889 | 121.0648 | 317.1536 | 13.91 |
| | Epitestosterone | C₁₉H₂₈O₂ | M + H | 289.21621 | 97.0648 | 109.0648 | 13.93 |
| | Estradiol benzoate | C₂₅H₂₈O₃ | M + H | 377.21112 | 279.1743 | 321.1849 | 12.22 |
| | Fludrocortisone acetate | C₂₃H₃₁FO₆ | M + H | 423.21774 | 181.1023 | 239.143 | 12.22 |
| | Flumetasone pivalate | C₂₇H₃₆F₂O₆ | M + H | 495.25527 | 57.0699 | 121.0648 | 15.25 |
| | Flumethasone | C₂₂H₂₈F₂O₅ | M + H | 411.19776 | 235.1117 | 277.1587 | 11.2 |
| | Fluocinolone acetonide | C₂₄H₃₀F₂O₆ | M + H | 453.20832 | 121.0648 | 337.1434 | 11.86 |
| | Fluorometholone | C₂₂H₂₉FO₄ | M + H | 377.21226 | 279.1743 | 135.0804 | 12.22 |
| | Flurandrenolide | C₂₄H₃₃FO₆ | M + H | 437.23339 | 361.1798 | 105.0699 | 12.01 |
| | Fluticasone propionate | C₂₅H₃₁F₃O₅S | M + H | 501.19171 | 121.0648 | 275.143 | 15.08 |
| | Nandrolone propionate | C₂₁H₃₀O₃ | M + H | 331.22677 | 257.18999 | 239.17943 | 17.38 |
| | Halcinonide | C₂₄H₃₂ClFO₅ | M + H | 455.19951 | 121.0648 | 377.1514 | 14.98 |
| | Dihydrodiethylstilbestrol | C₁₈H₂₂O₂ | M + H | 271.16926 | 109.1117 | 253.1587 | 11.92 |
| | Medroxyprogesterone | C₂₄H₃₄O₄ | M + H | 387.25299 | 123.0804 | 327.2319 | 15.81 |
| | acetate | | | | | | |
| | Megestrol acetate | C₂₄H₃₂O₄ | M + H | 385.23734 | 267.1743 | 224.156 | 15.53 |
| | Melengestrol acetate | C₂₅H₃₂O₄ | M + H | 397.23734 | 279.1743 | 337.2162 | 15.82 |
| | Methandienone | C₂₀H₂₈O₂ | M + H | 301.21621 | 283.20564 | 149.13248 | 12.73 |
| | Methylprednisolone | C₂₂H₃₀O₅ | M + H | 375.2166 | 161.0961 | 135.0804 | 10.99 |
| | Norethindrone | C₂₀H₂₆O₂ | M + H | 299.20056 | 109.0648 | 281.19 | 13.12 |
| | Nandrolone propionate | C₁₈H₂₆O₂ | M + H | 275.20056 | 109.06499 | 239.17885 | 12.47 |
| | Levonorgestrel | C₂₁H₂₈O₂ | M + H | 313.21621 | 109.0648 | 245.19 | 14.23 |
| | Prednicarbate | C₂₇H₃₆O₈ | M + H | 489.24829 | 147.0804 | 289.1587 | 14.91 |
| | Prednisolone | C₂₁H₂₈O₅ | M + H | 361.20095 | 147.0804 | 171.0804 | 10.2 |
| | Prednisone | C₂₁H₂₆O₅ | M + H | 359.1853 | 341.17474 | 237.12739 | 10.27 |
| | Progesterone | C₂₁H₃₀O₂ | M + H | 315.23186 | 109.06479 | 97.06479 | 15.76 |
| | Dehydrotestosterone | C₁₉H₂₆O₂ | M + H | 287.20056 | 121.0647 | 135.1167 | 12.22 |
| | Testosterone | C₁₉H₂₈O₂ | M + H | 289.21621 | 109.06479 | 97.06479 | 13.03 |
| | Trenbolone | C₁₈H₂₂O₂ | M + H | 271.16926 | 253.15869 | 199.11174 | 11.92 |
| | Triamcinolone | C₂₄H₃₁FO₆ | M + H | 435.21774 | 397.201 | 339.1591 | 11.69 |
| | acetonide | | | | | | |
| | Beclomethasone | C₂₂H₂₉ClO₅ | M + H | 409.17763 | 391.1671 | 279.1743 | 11.45 |
| | Betamethasone | C₂₈H₃₇FO₇ | M + H | 505.25961 | 279.1741 | 319.1693 | 15.45 |
| | dipropionate | | | | | | |
| | Betamethasone | C₂₇H₃₇FO₆ | M + H | 477.26469 | 279.1743 | 147.0804 | 14.55 |
| | valerate | | | | | | |
| | Betamethasone | C₂₂H₂₉FO₅ | M + H | 393.20718 | 355.19039 | 279.17434 | 11.14 |
| Nitroimidazoles | Dimetridazole | C₅H₇N₃O₂ | M + H | 142.0611 | 112.06311 | 95.06037 | 7.17 |
| | Ipronidazole | C₇H₁₁N₃O₂ | M + H | 170.0924 | 140.09441 | 123.09167 | 10.33 |
| | Metronidazole | C₆H₉N₃O₃ | M + H | 172.07167 | 128.04545 | 82.05255 | 6.39 |
| | Ornidazole | C₇H₁₀ClN₃O₃ | M + H | 220.04835 | 128.04547 | 203.14304 | 8.77 |
| | Hydroxydimetridazole | C₅H₇N₃O₃ | M + H | 158.05602 | 140.0455 | 110.0475 | 6.42 |
| | Ronidazole | C₆H₈N₄O₄ | M + H | 201.06183 | 140.04545 | 110.04746 | 7.03 |
| | Secnidazole | C₇H₁₁N₃O₃ | M + H | 186.08732 | 128.0455 | 111.0427 | 7.56 |
| | Tinidazole | C₈H₁₃N₃O₄S | M + H | 248.06995 | 154.0611 | 110.08384 | 8.12 |
| Antiviral | Amantadine | C₁₀H₁₇N | M + H | 152.14338 | 135.11683 | 107.08553 | 7.48 |
| drugs | Rimantadine; | C₁₂H₂₁N | M + H | 180.17468 | 163.14778 | 81.0704 | 9 |
| Lincosamides | Clindamycin | C₁₈H₃₃ClN₂O₅S | M + H | 425.18715 | 126.12773 | / | 8.69 |
| | Lincomycin | C₁₈H₃₄N₂O₆S | M + H | 407.221 | 126.12773 | 359.21766 | 6.76 |
| Amphenicols | Chloramphenicol | C₁₁H₁₂Cl₂N₂O₅ | M − H | 321.00505 | 257.03346 | 194.04588 | 10.11 |
| | Florfenicol | C₁₂H₁₄Cl₂FNO₄S | M − H | 355.99319 | 335.98696 | 185.02779 | 9.96 |
| | Thiamphenicol | C₁₂H₁₅Cl₂NO₅S | M − H | 353.99752 | 290.02593 | 185.02779 | 8.27 |
| β-Lactams | Ceftiofur | C₁₉H₁₇N₅O₇S₃ | M + H | 524.03629 | 241.03899 | 210.02065 | 9.63 |
| | Flucloxacillin | C₁₉H₁₇ClFN₃O₅S | M + H | 454.06342 | 160.04242 | 114.03742 | 12.5 |
| | Cloxacillin | C₁₉H₁₈ClN₃O₅S | M + H | 436.07285 | 277.0374 | 206.0367 | 12.26 |
| | Nafcillin | C₂₁H₂₂N₂O₅S | M + H | 415.13222 | 199.0754 | 171.0441 | 12.54 |
| | Piperacillin | C₂₃H₂₇N₅O₇S | M + H | 518.1704 | 143.0815 | 115.0502 | 10.16 |
| | Penicillin G | C₁₆H₁₈N₂O₄S | M + H | 335.106 | 217.06414 | 176.07061 | 10.77 |
| Quinoxalines | Quinocetone | C₁₈H₁₄N₂O₃ | M + H | 307.10772 | 143.06018 | 273.10178 | 11.99 |
| Antipyretics | Phenacetin | C₁₀H₁₃NO₂ | M + H | 180.10191 | 110.0602 | 138.09109 | 10.11 |
| and analgesics | Salicylic acid | C₇H₆O₃ | M − H | 137.02442 | 93.0346 | 65.0397 | 3.74 |
| | Amidopyrine | C₁₃H₁₇N₃O | M + H | 232.14444 | 113.10738 | 98.08418 | 6.46 |
| | Antipyrine | C₁₁H₁₂N₂O | M + H | 189.10224 | 147.09135 | 161.10696 | 8.39 |
| | Dapsone | C₁₂H₁₂N₂O₂S | M + H | 249.06922 | 156.01122 | 108.04431 | 9.03 |
| | Flunixin meglumine | C₁₄H₁₁F₃N₂O₂ | M + H | 297.08454 | 279.07397 | 264.0505 | 13.66 |
| | Acetaminophen | C₈H₉NO₂ | M + H | 152.07061 | 110.06004 | / | 6.25 |
| Psychoactive | Haloperidol | C₂₁H₂₃ClFNO₂ | M + H | 376.14741 | 358.13685 | 165.07102 | 10.3 |
| drugs | Imipramine | C₁₉H₂₄N₂ | M + H | 281.20123 | 86.0963 | 236.1434 | 10.71 |
| | Nitrazepam | C₁₅H₁₁N3O₃ | M + H | 282.08732 | 268.08383 | 207.09126 | 11.53 |
| | Oxazepam | C₁₅H₁₁ClN₂O₂ | M + H | 287.05818 | 241.05203 | 269.04703 | 11.49 |
| | Pemoline | C₉H₈N₂O₂ | M + H | 177.06585 | 106.0651 | 79.0542 | 7.74 |
| | Perphenazine | C₂₁H₂₆ClN₃OS | M + H | 404.15579 | 143.11763 | 171.14877 | 10.83 |
| | Promethazine | C₁₇H₂₀N₂S | M + H | 285.142 | 198.0372 | 86.09643 | 10.34 |
| | Propionylpromazine | C₂₀H₂₄N₂OS | M + H | 341.16821 | 86.0964 | 58.0651 | 10.95 |
| | hydrochloride | | | | | | |
| | Sulpiride | C₁₅H₂₃N₃O₄S | M + H | 342.1482 | 214.0169 | 112.1121 | 6.21 |
| | Xylazine | C₁₂H₁₆N₂S | M + H | 221.1107 | 90.0372 | 164.0528 | 8.27 |
| | hydrochloride | | | | | | |
| | Acepromazin | C₁₉H₂₂N₂OS | M + H | 327.15256 | 254.06341 | 86.09643 | 10.2 |
| | Anisopirol | C₁₉H₂₄FN₃O | M + H | 330.19762 | 312.1865 | 121.076 | 7.44 |
| | Azaperone | C₁₉H₂₂FN₃O | M + H | 328.18197 | 165.07069 | 121.07603 | 8.04 |
| | Carbamazepine | C₁₅H₁₂N₂O | M + H | 237.10224 | 194.0964 | 220.0757 | 11.15 |
| | Chloropromazine | C₁₇H₁₉ClN₂S | M + H | 319.10302 | 86.09643 | 246.01379 | 11.25 |
| | Diazepam | C₁₆H₁₃ClN₂O | M + H | 285.07892 | 154.0415 | 193.0883 | 13.58 |
| | Diphenhydramine | C₁₇H₂₁NO | M + H | 256.16959 | 167.0855 | 152.0621 | 10.03 |
| | hydrochloride | | | | | | |
| | Droperidol | C₂₂H₂₂FN₃O₂ | M + H | 380.17688 | 165.07072 | 194.09726 | 9.35 |
| | Estazolam | C₁₆H₁₁ClN₄ | M + H | 295.0745 | 267.05524 | 205.0755 | 11.44 |
| Macrolides | Clarithromycin | C₃₈H₆₉NO₁₃ | M + H | 748.48417 | 158.11756 | 116.07061 | 11.04 |
| | Tiamulin | C₂₈H₄₇NO₄S | M + H | 494.32986 | 192.10528 | 119.01613 | 10.8 |
| | Erythromycin | C₃₇H₆₇NO₁₃ | M + H | 734.46852 | 158.11756 | 116.07061 | 9.92 |
| | Kitasamycin | C₄₀H₆₇NO₁₄ | M + H | 786.46343 | 558.32727 | 174.11247 | 11.25 |
| | Tilmicosin | C₄₆H₈₀N₂O₁₃ | M + H | 869.57332 | 174.11247 | 88.07569 | 8.93 |
| | Tylosin | C₄₆H₇₇NO₁₇ | M + H | 916.52643 | 772.44778 | 174.11247 | 10.17 |
| anthelmintics | Mebendazole | C₁₆H₁₃N₃O₃ | M + H | 296.10297 | 264.07675 | 105.03349 | 10.85 |
| | Mebendazole-amine | C₁₄H₁₁N₃O | M + H | 238.09749 | 105.03369 | / | 8.45 |
| | 2-aminofluorobenzimidazole | C₁₄H₁₀FN₃O | M + H | 256.08807 | 123.0241 | 113.0397 | 8.74 |
| | Oxfendazole | C₁₅H₁₃N₃O₃S | M + H | 316.07504 | 284.04882 | 223.0576 | 9.26 |
| | Oxibendazole | C₁₂H₁₅N₃O₃ | M + H | 250.11862 | 218.0924 | 176.04545 | 9.33 |
| | Parbendazole | C₁₃H₁₇N₃O₂ | M + H | 248.13935 | 216.1131 | 160.0505 | 10.65 |
| | 5-hydroxythiabendazole | C₁₀H₇N₃OS | M + H | 218.03826 | 191.02736 | / | 6.44 |
| | Tiabendazole | C₁₀H₇N₃S | M + H | 202.04334 | 175.03245 | 131.06037 | 7.12 |
| | Albendazole | C₁₂H₁₅N₃O₂S | M + H | 266.09577 | 234.06956 | 191.01478 | 10.99 |
| | Albendazole sulfone | C₁₂H₁₅N₃O₄S | M + H | 298.0856 | 266.05939 | 224.01244 | 9.4 |
| | Albendazole sulfoxide | C₁₂H₁₅N₃O₃S | M + H | 282.09069 | 240.04347 | 208.01752 | 8.08 |
| | Amino albendazole | C₁₀H₁₃N₃O₂S | M + H | 240.08012 | 198.03317 | 165.05328 | 6.94 |
| | sulfone | | | | | | |
| | Benzimidazole | C₇H₆N₂ | M + H | 119.06037 | 92.0495 | 65.0386 | 4.8 |
| | Febantel | C₂₀H₂₂N₄O₆S | M + H | 447.13328 | 383.08085 | 280.05391 | 14.78 |
| | Lobendazole | C₁₀H₁₁N₃O₂ | M + H | 206.0924 | 160.0505 | 178.0609 | 7.59 |
| | toltrazuril sulfoxide | C₁₈H₁₄F₃N₃O₅S | M − H | 440.05335 | 371.05813 | / | 8.97 |
| | Clopidol | C₇H₇Cl₂NO | M + H | 191.99775 | 86.9996 | 101.01525 | 6.83 |
| | Dinitolmide | C₈H₇N₃O₅ | M − H | 224.03129 | 181.02438 | 77.03858 | 9.91 |
| | Halofuginone | C₁₆H₁₇BrClN₃O₃ | M + H | 414.02146 | 100.07598 | 120.08076 | 8.89 |
| | Levamisole | C₁₁H₁₂N₂S | M + H | 205.0794 | 178.0685 | / | 6.99 |
| Pesticides | Buprofezin | C₁₆H₂₃N₃OS | M + H | 306.16346 | 201.10561 | 116.05285 | 17.73 |
| | Carboxin | C₁₂H₁₃NO₂S | M + H | 236.07398 | 143.01613 | 93.0573 | 12.61 |
| | 3-hydroxy carbofuran | C₁₂H₁₅NO₄ | M + H | 238.10738 | 163.07536 | 135.08044 | 9.09 |
| | Clothianidin | C₆H₈ClN₅O₂S | M + H | 250.016 | 131.96692 | 113.0168 | 8.96 |
| | Cyromazine | C₆H₁₀N₆ | M + H | 167.10397 | 85.05087 | 125.08217 | 3.63 |
| | Diuron | C₉H₁₀Cl₂N₂O | M + H | 233.02429 | 72.04439 | 159.97153 | 12.65 |
| | Ethopabate | C₁₂H₁₅NO₄ | M + H | 238.10738 | 206.08117 | 164.07061 | 10.29 |
| | Fipronil | C₁₂H₄Cl₂F₆N₄OS | M − H | 434.93143 | 329.95845 | 249.95853 | 15.66 |
| | Fluridone | C₁₉H₁₄F₃NO | M + H | 330.11003 | 310.1038 | 290.09757 | 13.62 |
| | Acephate | C₄H₁₀NO₃PS | M + H | 184.01918 | 142.99264 | 112.99968 | 5.61 |
| | Hexazinone | C₁₂H₂₀N₄O₂ | M + H | 253.1659 | 171.08765 | 71.06037 | 10.59 |
| | Imazalil | C₁₄H₁₄C₁₂N₂O | M + H | 297.0556 | 158.97628 | 255.00864 | 10.77 |
| | Imidacloprid | C₉H₁₀ClN₅O₂ | M + H | 256.05958 | 209.05885 | 175.09793 | 9.3 |
| | Linuron | C₉H₁₀Cl₂N₂O₂ | M + H | 249.01921 | 159.97153 | 182.02414 | 14.09 |
| | Metribuzin | C₈H₁₄N₄OS | M + H | 215.09611 | 187.10119 | 131.03859 | 11.73 |
| | Myclobutanil | C₁₅H₁₇ClN₄ | M + H | 289.12145 | 70.03997 | 125.01525 | 14.21 |
| | Acetamiprid | C₁₀H₁₁ClN₄ | M + H | 223.0745 | 126.0105 | 98.9997 | 9.58 |
| | Norflurazon | C₁₂H₉ClF₃N₃O | M + H | 304.0459 | 284.03967 | 160.03686 | 12.81 |
| | Propyzamide | C₁₂H₁₁Cl₂NO | M + H | 256.02905 | 189.9821 | 172.95555 | 14.68 |
| | Atrazine | C₈H₁₄ClN₅ | M + H | 216.10105 | 174.0541 | 96.05562 | 12.63 |
| | Simazine | C₇H₁₂ClN₅ | M + H | 202.0854 | 132.0323 | 104.001 | 11.35 |
| | Desethyl atrazine | C₆H₁₀ClN₅ | M + H | 188.06975 | 146.0228 | 104.001 | 9.37 |
| | Azoxystrobin | C₂₂H₁₇N₃O₅ | M + H | 404.1241 | 344.10397 | 329.07949 | 14.2 |
| | Benoxacor | C₁₁H₁₁Cl₂NO₂ | M + H | 260.02396 | 149.08352 | 120.04439 | 14.31 |
| Other | Scopolamine | C₁₇H₂₁NO₄ | M + H | 304.15433 | 156.10191 | 138.09134 | 7 |
| substances | Valnemulin | C₃₁H₅₂N₂O₅S | M + H | 565.36697 | 263.1417 | 72.08078 | 11.15 |
| | Atropine | C₁₇H₂₃NO₃ | M + H | 290.17507 | 124.11208 | 93.06988 | 7.61 |

### 5. Sample Analysis

The filtrate obtained in step (2.3) was analyzed by ultra high performance liquid chromatography-quadrupole/electrostatic field orbitrap high resolution mass spectrometer to obtain original data of the sample.

### 6. Compound Screening

The original data of the sample was automatically extracted within a mass window of 5 ppm using the Trace Finder software, where when a compound met the following conditions at the same time, it indicated that the compound was a suspected positive compound in the sample:

(i) a signal-noise ratio (S/N) is greater than 3;

(ii) a difference between the measured retention time and the corresponding retention time in the mass spectrometry database is <0.2 min or within ±2.5% (no more than 0.5 min);

(iii) the measured parent ion and one fragment ion simultaneously matched the corresponding information in the mass spectrometry database, and a mass accuracy of the parent ion was less than 5 ppm and a mass accuracy of the fragment ion was less than 10 ppm.

### 7. Confirmation

A fragment ion mass spectrometry of the suspected positive compound in the sample was compared with a fragment ion mass spectrometry of standard for further identification, where if the mass accuracy between main fragment ions in the two fragment ion mass spectrometries is less than 10 ppm and the main fragment ions in the two fragment ion mass spectrometries have identical relative abundance, it confirmed that the suspected positive compound was present in the sample.

### 8. Sensitivity Analysis

The detection limit of each compound of the veterinary drugs and additives in the invention was the lowest concentration that the compound can be detected in the sample in the screening in step (6) and in the confirmation in step (7). Further, the detection limits of the veterinary drugs and additives were shown in Table 6.

The parent ion extraction chromatograms of reference standards of the veterinary drugs and additives were shown in FIGS. 4-210, where a concentration of each reference standard was 10 ng/mL.

### 9. Method Validation

It can be seen from above that the method of the invention mainly achieved the screening and confirmation of compounds, that was, this method was suitable for the qualification of the residues in the animal-derived food.

Therefore, the validity of the method of the invention was assessed mainly in terms of detection limit, recovery rate, precision and matrix effect.

9.1 Detection Limit

The blank sample was processed in the same manner and then analyzed. The measurement results showed that the blank sample has no interference with the drug to be tested at the corresponding retention time. If the blank sample had a peak at the retention time of the drug to be tested, the value of the blank sample should be deducted before the calculation.

An appropriate amount of the mixed standard solution was added into 5 g of the blank matrix, which was then processed by the above pretreatment method and analyzed. The detection limit of each compound in chicken, pork, mutton, beef, chicken eggs, duck eggs, cow's milk and goat's milk was determined according to the conditions of the compound confirmation. The results were shown in Table 6.

9.2 Recovery Rate and Precision

A standard solution having a concentration near the detection limit was added to the blank sample according to the spiking method. 5 parallel samples of each concentration were tested 3 times for the calculation of intra- and inter-batch relative standard deviations (RSD). The results were shown in Table 6, in which the recovery rates of the 207 veterinary drugs and additives were all in the range of 40%-130%, and intra- and inter-batch relative standard deviations were less than 30%.

TABLE 6
Recovery rate and precision of the method of the invention

| | Average recovery rate (n = 5) | | | | | | | | Intra-batch RSD (n = 5) | | | | | | | | Detection |
|---|---|---|---|---|---|---|---|---|---|---|---|---|---|---|---|---|---|
| | Chicken | Duck | | | | | Cow's | goat's | Chicken | Duck | | | | | Cow's | Goat's | limit |
| Compound | egg | egg | Chicken | Pork | Mutton | Beef | milk | milk | egg | egg | Chicken | Pork | Mutton | Beef | milk | milk | μg/kg |
| Sulfamethazine | 107.33 | 105.30 | 117.10 | 100.00 | 118.19 | 106.94 | 97.81 | 115.59 | 5.71 | 12.84 | 16.03 | 12.63 | 16.77 | 12.37 | 9.29 | 6.03 | 5 |
| Sulformethoxine | 116.53 | 97.62 | 123.57 | 110.26 | 98.63 | 125.30 | 84.87 | 103.92 | 3.43 | 11.44 | 12.46 | 8.38 | 4.18 | 11.28 | 7.00 | 4.70 | 5 |
| Sulfisoxazole | 108.13 | 102.88 | 126.71 | 72.86 | 97.80 | 119.28 | 84.40 | 108.11 | 6.29 | 19.23 | 10.49 | 5.06 | 3.61 | 11.86 | 7.07 | 5.89 | 5 |
| Sulfaguanidine | 75.37 | 68.11 | 91.07 | 72.86 | 63.90 | 78.64 | 63.44 | 81.39 | 3.78 | 16.56 | 11.38 | 5.06 | 4.28 | 9.98 | 3.64 | 7.88 | 5 |
| Sulfamerazine | 109.57 | 105.62 | 106.40 | 106.05 | 125.02 | 109.46 | 96.08 | 109.08 | 3.81 | 12.37 | 13.09 | 11.80 | 10.03 | 11.75 | 14.49 | 6.65 | 5 |
| Sulfamethylthiadizaole | 107.15 | 89.54 | 121.58 | 117.05 | 92.51 | 109.88 | 75.09 | 98.90 | 4.59 | 18.76 | 13.89 | 8.33 | 2.62 | 11.99 | 12.23 | 9.12 | 5 |
| Sulfamethoxazole | 119.91 | 97.62 | 129.73 | 98.24 | 100.92 | 125.51 | 85.06 | 105.22 | 4.77 | 14.68 | 10.35 | 19.89 | 2.25 | 9.53 | 8.19 | 6.58 | 5 |
| Sulfamethoxydiazine | 108.36 | 97.01 | 110.06 | 98.24 | 115.37 | 103.30 | 114.77 | 113.79 | 2.72 | 16.20 | 10.84 | 19.89 | 14.99 | 11.90 | 11.70 | 7.86 | 5 |
| Sulfamethoxypyridazine | 108.36 | 97.01 | 110.88 | 110.38 | 115.37 | 103.30 | 128.93 | 113.79 | 2.72 | 16.20 | 19.33 | 13.28 | 14.99 | 11.90 | 22.57 | 9.40 | 5 |
| Sulfamonomethoxine | 116.02 | 100.53 | 127.83 | 81.00 | 97.78 | 121.97 | 81.79 | 99.84 | 4.66 | 13.33 | 10.24 | 1.87 | 3.08 | 10.42 | 7.51 | 16.23 | 5 |
| Sulfamethazole | 99.83 | 87.29 | 121.09 | 115.70 | 99.41 | 102.51 | 77.41 | 101.33 | 6.21 | 14.97 | 18.16 | 5.97 | 3.82 | 19.25 | 7.67 | 6.82 | 5 |
| Sulfapyridinee | 104.31 | 94.58 | 113.58 | 95.80 | 101.39 | 110.11 | 85.51 | 97.81 | 4.67 | 16.05 | 9.55 | 11.43 | 4.24 | 10.83 | 7.54 | 9.43 | 5 |
| Sulfaquinoxaline | 112.49 | 91.75 | 126.78 | 96.35 | 96.56 | 126.50 | 80.37 | 96.99 | 4.01 | 15.32 | 13.79 | 3.97 | 5.99 | 10.00 | 15.77 | 4.43 | 5 |
| Sulfathiazole | 104.11 | 96.65 | 117.51 | 95.17 | 101.83 | 104.97 | 81.59 | 97.61 | 5.05 | 16.86 | 12.34 | 1.98 | 3.22 | 13.87 | 7.35 | 8.25 | 5 |
| Sulfisomidine | 92.51 | 88.80 | 110.79 | 112.53 | 86.99 | 96.77 | 75.92 | 80.19 | 3.83 | 17.32 | 11.92 | 3.02 | 3.58 | 6.41 | 7.41 | 4.63 | 5 |
| Trimethoprim | 91.75 | 91.22 | 125.59 | 115.59 | 87.44 | 104.09 | 76.71 | 75.08 | 3.30 | 4.53 | 13.56 | 2.26 | 3.32 | 4.05 | 2.21 | 1.29 | 5 |
| Diaveridine | 85.49 | 84.27 | 107.60 | 111.96 | 87.36 | 79.32 | 77.69 | 74.87 | 3.72 | 5.52 | 7.36 | 2.75 | 2.48 | 4.88 | 2.82 | 1.40 | 5 |
| Phthalylsulfathiazole | 76.52 | 83.83 | 92.73 | 103.23 | 47.84 | 94.81 | 71.74 | 76.23 | 10.73 | 17.398 | 1.32 | 4.53 | 6.96 | 5.97 | 12.26 | 12.61 | 5 |
| Sulfabenzamide | 115.96 | 104.82 | 91.52 | 116.62 | 88.21 | 123.29 | 79.44 | 103.09 | 1.35 | 18.82 | 1.26 | 3.22 | 3.72 | 8.02 | 7.19 | 7.41 | 5 |
| Sulfacetamide | 101.31 | 101.84 | 82.67 | 115.32 | 95.93 | 114.60 | 85.38 | 105.49 | 4.83 | 13.11 | 4.57 | 4.54 | 2.24 | 7.02 | 6.98 | 3.94 | 5 |
| Sulfachlorpyridazine | 108.98 | 100.48 | 119.07 | 107.63 | 95.52 | 121.83 | 86.27 | 102.72 | 2.52 | 18.21 | 10.97 | 5.79 | 2.30 | 10.80 | 9.69 | 5.88 | 5 |
| Sulfachloropyrazine | 117.78 | 123.57 | 120.41 | 102.16 | 95.45 | 129.02 | 82.42 | 106.08 | 12.26 | 12.54 | 10.07 | 5.78 | 4.31 | 7.29 | 13.04 | 9.33 | 10 |
| Sulfadiazine | 99.39 | 94.53 | 110.89 | 89.61 | 98.44 | 111.94 | 82.01 | 97.77 | 5.54 | 14.84 | 11.93 | 3.30 | 3.60 | 11.08 | 7.74 | 4.55 | 5 |
| Sulfadimethoxine | 111.07 | 95.77 | 91.87 | 82.67 | 96.41 | 126.47 | 108.85 | 112.35 | 3.80 | 17.47 | 10.38 | 1.84 | 5.86 | 11.93 | 10.18 | 14.32 | 10 |
| Lomefloxacin | 81.03 | 80.48 | 107.61 | 117.37 | 67.89 | 99.85 | 53.42 | 68.06 | 2.47 | 6.25 | 10.08 | 6.24 | 3.71 | 5.29 | 22.46 | 10.09 | 5 |
| Marbofloxacin | 72.04 | 71.06 | 129.40 | 75.49 | 60.31 | 87.45 | 59.37 | 66.48 | 5.56 | 4.77 | 11.01 | 2.89 | 2.09 | 3.90 | 3.89 | 9.43 | 5 |
| Nadifloxacin | 109.80 | 103.73 | 88.75 | 122.99 | 97.52 | 117.22 | 82.32 | 103.66 | 2.19 | 12.67 | 1.54 | 2.59 | 4.58 | 7.78 | 16.72 | 5.42 | 5 |
| Nalidixic acid | 111.07 | 113.15 | 78.28 | 58.85 | 108.02 | 105.39 | 89.80 | 106.37 | 2.20 | 3.43 | 3.65 | 2.04 | 5.18 | 3.68 | 2.85 | 13.14 | 5 |
| Norfloxacin | 55.82 | 52.32 | 121.67 | 81.51 | 47.42 | 66.71 | 42.07 | 50.43 | 4.24 | 7.88 | 1.95 | 1.49 | 2.97 | 3.39 | 9.12 | 12.93 | 5 |
| Ofloxacin | 77.45 | 75.22 | 122.19 | 100.00 | 65.33 | 91.24 | 61.95 | 69.12 | 4.19 | 5.49 | 1.80 | 6.85 | 1.85 | 4.79 | 6.71 | 7.54 | 5 |
| Orbifloxacin | 97.83 | 100.86 | 55.97 | 81.00 | 88.97 | 116.12 | 85.47 | 85.89 | 4.36 | 7.13 | 1.71 | 3.17 | 1.34 | 4.69 | 4.23 | 4.33 | 1 |
| Oxolinic acid | 117.79 | 112.71 | 81.36 | 100.28 | 121.73 | 123.19 | 89.48 | 123.61 | 3.95 | 6.79 | 4.99 | 2.70 | 7.34 | 6.63 | 16.68 | 15.01 | 5 |
| Pefloxacin | 79.46 | 74.07 | 106.42 | 100.82 | 69.26 | 88.41 | 60.23 | 68.41 | 5.96 | 5.18 | 0.94 | 4.00 | 2.72 | 3.77 | 10.37 | 15.41 | 5 |
| Cinoxacin | 101.39 | 101.45 | 104.66 | 97.16 | 66.25 | 116.45 | 86.01 | 96.84 | 4.56 | 5.24 | 1.79 | 13.24 | 3.20 | 6.56 | 4.25 | 7.31 | 1 |
| Sarafloxacin | 103.64 | 96.36 | 106.00 | 66.16 | 96.00 | 117.72 | 70.59 | 79.18 | 2.51 | 8.15 | 3.36 | 2.49 | 2.54 | 6.55 | 4.59 | 11.31 | 5 |
| Sparfloxacin | 101.80 | 99.26 | 106.79 | 107.72 | 98.31 | 113.40 | 81.74 | 86.09 | 2.49 | 6.89 | 1.92 | 6.45 | 2.46 | 6.30 | 7.70 | 7.61 | 5 |
| Ciprofloxacin | 66.44 | 62.44 | 64.77 | 54.72 | 52.07 | 77.06 | 52.11 | 57.55 | 4.93 | 5.07 | 2.10 | 3.20 | 4.76 | 3.39 | 7.87 | 10.96 | 5 |
| Danofloxacin | 76.80 | 72.83 | 82.61 | 82.80 | 73.30 | 92.56 | 63.97 | 77.47 | 3.78 | 5.14 | 2.81 | 2.52 | 5.85 | 6.20 | 13.03 | 20.87 | 5 |
| Difloxacin | 106.01 | 98.81 | 119.85 | 115.01 | 109.12 | 123.33 | 89.41 | 96.66 | 2.48 | 7.11 | 3.00 | 3.51 | 1.94 | 5.84 | 9.02 | 15.08 | 5 |
| Enoxacin | 53.36 | 49.39 | 55.32 | 96.84 | 41.87 | 64.95 | 57.74 | 62.21 | 6.56 | 6.10 | 3.17 | 4.62 | 2.44 | 4.17 | 9.37 | 7.35 | 10 |
| Enrofloxacin | 98.69 | 92.73 | 98.13 | 84.62 | 89.95 | 110.79 | 71.81 | 81.96 | 3.46 | 5.73 | 1.98 | 1.66 | 2.87 | 4.99 | 4.87 | 16.67 | 5 |
| Fleroxacin | 82.81 | 82.61 | 84.37 | 88.46 | 63.08 | 97.19 | 67.11 | 74.19 | 5.42 | 5.44 | 3.28 | 7.26 | 2.18 | 5.44 | 5.62 | 7.05 | 5 |
| Pipemidic acid | 40.02 | 43.2 | 41.06 | 40.02 | 42.56 | 44.89 | 36.54 | 37.85 | 5.69 | 3.25 | 4.17 | 9.56 | 1.25 | 7.25 | 5.14 | 6.03 | 5 |
| Flumequine | 116.54 | 111.57 | 121.92 | 89.19 | 98.06 | 116.60 | 85.17 | 101.03 | 3.15 | 5.61 | 2.35 | 13.65 | 11.95 | 5.24 | 12.89 | 11.66 | 5 |
| Gatifloxacin | 96.49 | 93.81 | 96.60 | 70.51 | 83.92 | 111.58 | 77.44 | 78.67 | 4.61 | 3.67 | 2.62 | 2.08 | 1.52 | 3.16 | 14.34 | 9.29 | 1 |
| Mebendazole | 106.13 | 89.62 | 128.31 | 113.01 | 81.66 | 114.52 | 84.30 | 83.34 | 4.36 | 6.89 | 3.41 | 15.10 | 10.55 | 4.55 | 8.36 | 13.33 | 10 |
| Mebendazole-amine | 95.77 | 87.82 | 94.10 | 95.78 | 124.23 | 102.95 | 79.34 | 82.26 | 3.72 | 10.73 | 2.59 | 7.80 | 2.53 | 4.05 | 15.75 | 8.14 | 5 |
| 2-Aminofluorobenzimidazole | 101.04 | 87.96 | 99.54 | 119.45 | 115.02 | 102.55 | 87.58 | 87.19 | 2.18 | 13.35 | 2.32 | 3.86 | 4.35 | 5.54 | 16.02 | 8.32 | 5 |
| Oxfendazole | 116.58 | 112.57 | 122.94 | 116.81 | 103.29 | 127.73 | 88.63 | 100.73 | 2.73 | 8.15 | 1.75 | 3.85 | 2.34 | 3.82 | 10.95 | 18.85 | 5 |
| Oxibendazole | 117.17 | 107.42 | 122.48 | 68.29 | 105.56 | 125.21 | 86.64 | 91.46 | 1.63 | 10.45 | 3.82 | 18.13 | 5.79 | 3.82 | 14.81 | 13.35 | 5 |
| Parbendazole | 111.03 | 86.08 | 126.67 | 117.72 | 48.78 | 127.27 | 96.08 | 80.95 | 1.82 | 11.65 | 6.97 | 1.78 | 13.29 | 3.91 | 8.32 | 4.23 | 10 |
| 5-Hydroxythiabendazole | 118.25 | 98.67 | 104.65 | 100.02 | 99.40 | 105.76 | 87.83 | 87.72 | 6.70 | 10.65 | 2.75 | 4.01 | 7.84 | 5.08 | 7.29 | 6.76 | 1 |
| Tiabendazole | 113.43 | 105.37 | 114.61 | 76.46 | 106.20 | 49.93 | 85.37 | 83.94 | 3.36 | 8.21 | 2.35 | 1.88 | 2.95 | 4.64 | 16.74 | 8.36 | 1 |
| Albendazole | 80.66 | 87.93 | 125.10 | 100.31 | 54.60 | 114.37 | 80.74 | 79.71 | 15.44 | 13.00 | 7.51 | 1.32 | 15.69 | 7.35 | 10.13 | 8.62 | 10 |
| Albendazole sulfone | 118.53 | 112.57 | 124.94 | 119.77 | 106.23 | 110.67 | 89.06 | 100.30 | 1.64 | 5.67 | 1.64 | 15.13 | 1.81 | 4.56 | 3.95 | 8.60 | 5 |
| Albendazole | 111.30 | 105.67 | 120.21 | 63.20 | 102.28 | 121.43 | 101.04 | 95.93 | 4.93 | 3.74 | 1.52 | 6.09 | 3.47 | 4.05 | 17.02 | 11.97 | 5 |
| sulfoxide | | | | | | | | | | | | | | | | | |
| Amino albendazole | 95.20 | 92.47 | 91.10 | 84.41 | 95.20 | 96.86 | 82.79 | 86.35 | 4.12 | 6.65 | 1.09 | 2.83 | 1.33 | 4.12 | 1.44 | 3.66 | 5 |
| sulfone | | | | | | | | | | | | | | | | | |
| Benzimidazole | 93.54 | 87.61 | 90.77 | 86.90 | 91.85 | 97.27 | 40.21 | 83.30 | 3.02 | 7.72 | 1.42 | 4.19 | 1.67 | 3.97 | 4.55 | 2.59 | 5 |
| Febantel | 114.08 | 116.87 | 129.99 | 107.65 | 110.37 | 114.90 | 87.18 | 87.75 | 5.09 | 9.67 | 3.66 | 6.36 | 9.37 | 7.59 | 5.19 | 10.81 | 5 |
| Lobendazole | 113.88 | 110.35 | 123.01 | 106.47 | 112.90 | 129.35 | 98.20 | 93.69 | 1.64 | 5.86 | 6.77 | 5.49 | 2.75 | 4.23 | 8.63 | 16.17 | 1 |
| Cyproheptadine | 122.94 | 96.21 | 93.91 | 49.01 | 109.85 | 129.61 | 86.36 | 84.65 | 5.99 | 12.94 | 5.77 | 3.25 | 4.86 | 4.28 | 6.98 | 6.90 | 5 |
| Fenoterol | 97.17 | 89.27 | 112.96 | 119.51 | 98.65 | 91.59 | 74.73 | 87.81 | 9.31 | 7.78 | 3.50 | 5.04 | 4.22 | 5.89 | 7.71 | 15.34 | 1 |
| Labetalol | 102.47 | 100.02 | 122.06 | 65.71 | 119.16 | 117.35 | 89.16 | 86.51 | 3.28 | 9.09 | 3.02 | 1.89 | 3.94 | 4.21 | 6.79 | 7.38 | 1 |
| Penbutolol | 105.46 | 93.64 | 122.27 | 114.46 | 99.28 | 107.23 | 74.30 | 92.45 | 3.78 | 11.03 | 3.05 | 5.26 | 5.05 | 4.11 | 4.41 | 4.24 | 5 |
| Phenylethanolamine A | 113.04 | 106.47 | 109.98 | 118.25 | 122.10 | 126.13 | 90.23 | 87.01 | 4.02 | 9.73 | 1.37 | 12.55 | 2.41 | 3.91 | 7.04 | 6.12 | 5 |
| Ractopamine | 102.78 | 101.49 | 49.70 | 76.43 | 124.32 | 115.85 | 78.83 | 77.76 | 1.41 | 7.03 | 1.49 | 9.07 | 4.94 | 6.28 | 10.50 | 21.52 | 1 |
| Salbutamol | 48.61 | 43.56 | 50.98 | 84.95 | 45.18 | 55.02 | 48.61 | 53.92 | 4.94 | 4.95 | 5.15 | 2.08 | 2.01 | 3.90 | 3.26 | 4.61 | 5 |
| Salmeterol | 108.40 | 87.00 | 68.31 | 99.34 | 84.56 | 129.93 | 83.92 | 92.70 | 1.24 | 19.62 | 1.02 | 2.13 | 8.69 | 5.38 | 11.07 | 16.16 | 5 |
| Terbutaline | 63.33 | 59.54 | 117.89 | 107.38 | 56.94 | 73.44 | 58.44 | 69.08 | 3.86 | 5.97 | 2.26 | 5.44 | 2.05 | 3.22 | 3.41 | 3.70 | 1 |
| Mabuterol | 109.36 | 112.98 | 110.45 | 102.99 | 120.99 | 127.69 | 90.97 | 80.99 | 4.06 | 4.47 | 1.89 | 3.98 | 1.20 | 4.30 | 4.99 | 6.38 | 1 |
| Tulobuterol | 99.90 | 101.55 | 109.70 | 95.59 | 107.92 | 119.79 | 98.20 | 85.74 | 1.97 | 3.55 | 0.89 | 16.63 | 2.93 | 4.54 | 9.45 | 4.37 | 1 |
| Bambuterol | 105.10 | 108.19 | 113.98 | 78.27 | 109.55 | 127.02 | 40.87 | 83.69 | 2.22 | 3.61 | 1.50 | 3.88 | 6.37 | 5.31 | 8.18 | 8.55 | 5 |
| Brombuterol | 101.59 | 104.56 | 111.96 | 91.84 | 116.26 | 115.00 | 89.10 | 83.95 | 3.02 | 2.88 | 2.49 | 2.86 | 5.42 | 7.61 | 10.01 | 17.06 | 5 |
| Cimaterol | 78.22 | 75.90 | 87.06 | 95.31 | 74.56 | 91.23 | 69.93 | 76.73 | 3.47 | 9.41 | 2.78 | 7.62 | 2.40 | 3.73 | 2.83 | 3.33 | 5 |
| Cimbuterol | 93.45 | 88.62 | 109.10 | 99.61 | 90.91 | 106.20 | 82.09 | 83.69 | 2.89 | 7.62 | 2.96 | 4.63 | 4.17 | 3.89 | 4.89 | 4.61 | 1 |
| Clenbuterol | 104.64 | 104.33 | 109.44 | 105.20 | 115.41 | 114.84 | 102.37 | 90.63 | 2.67 | 6.52 | 1.34 | 18.25 | 1.55 | 2.66 | 4.80 | 9.55 | 1 |
| Clenproperol | 97.76 | 103.92 | 107.58 | 108.68 | 106.72 | 116.96 | 89.37 | 84.62 | 1.78 | 6.28 | 1.74 | 8.19 | 1.77 | 1.86 | 5.95 | 4.34 | 1 |
| Clonidine | 89.81 | 101.20 | 103.16 | 82.21 | 94.07 | 118.26 | 85.76 | 83.82 | 3.24 | 4.32 | 3.60 | 5.71 | 1.82 | 4.05 | 2.76 | 2.75 | 1 |
| Chlormadinone | 78.75 | 129.62 | 73.45 | 107.77 | 86.16 | 129.49 | 101.27 | 92.70 | 8.25 | 19.87 | 5.71 | 6.50 | 6.89 | 3.36 | 9.61 | 8.26 | 50 |
| acetate | | | | | | | | | | | | | | | | | |
| 17a-Methyltestosterone | 102.28 | 109.75 | 122.00 | 116.22 | 102.18 | 129.58 | 96.14 | 90.16 | 4.31 | 9.17 | 2.34 | 4.39 | 2.98 | 4.35 | 7.58 | 12.18 | 10 |
| Clobetasol | 102.11 | 126.78 | 128.38 | 101.60 | 119.61 | 118.46 | 95.81 | 90.12 | 8.07 | 6.07 | 4.68 | 7.72 | 10.42 | 4.99 | 5.24 | 9.15 | 10 |
| propionate | | | | | | | | | | | | | | | | | |
| Clobetasone butyrate | 89.10 | 127.99 | 96.81 | 121.04 | 117.08 | 119.22 | 119.05 | 130.00 | 14.71 | 8.62 | 8.15 | 3.48 | 12.46 | 5.94 | 9.95 | 8.99 | 50 |
| Corticosterone | 104.54 | 112.30 | 121.64 | 104.67 | 104.70 | 126.14 | 88.66 | 93.38 | 9.70 | 4.44 | 3.11 | 3.67 | 1.91 | 4.75 | 11.10 | 8.69 | 5 |
| Hydrocortisone | 102.37 | 115.69 | 116.91 | 103.92 | 97.81 | 118.04 | 87.45 | 97.98 | 3.90 | 8.46 | 1.90 | 5.38 | 1.23 | 4.14 | 4.57 | 5.35 | 5 |
| Cortisone acetate | 106.52 | 122.12 | 123.53 | 123.50 | 41.24 | 86.49 | 94.96 | 85.53 | 7.01 | 8.77 | 2.52 | 5.95 | 10.12 | 7.37 | 5.86 | 7.72 | 50 |
| Cortisone | 107.17 | 101.68 | 123.26 | 108.89 | 102.05 | 121.62 | 107.72 | 97.72 | 9.45 | 9.75 | 1.30 | 9.83 | 2.77 | 4.34 | 3.31 | 2.51 | 10 |
| Deflazacort | 100.00 | 112.58 | 117.92 | 116.69 | 42.34 | 104.06 | 88.44 | 92.51 | 2.41 | 3.16 | 2.51 | 2.29 | 4.18 | 6.43 | 3.20 | 3.48 | 5 |
| Dexamethasone | 99.67 | 113.52 | 121.99 | 110.12 | 108.49 | 124.95 | 87.95 | 90.08 | 3.37 | 6.00 | 1.53 | 4.61 | 1.77 | 3.43 | 9.03 | 12.72 | 5 |
| Diflorasone | 106.11 | 117.12 | 126.18 | 111.55 | 89.49 | 126.41 | 102.46 | 92.73 | 6.68 | 14.10 | 3.44 | 15.34 | 8.07 | 5.33 | 3.79 | 9.87 | 10 |
| diacetate | | | | | | | | | | | | | | | | | |
| Epitestosterone | 95.30 | 115.25 | 123.78 | 113.47 | 111.03 | 128.48 | 96.72 | 87.38 | 6.05 | 6.33 | 1.82 | 5.20 | 1.88 | 3.62 | 8.39 | 10.37 | 10 |
| Estradiol benzoate | 103.64 | 85.86 | 122.19 | 120.55 | 103.86 | 127.50 | 87.18 | 100.73 | 3.59 | 17.18 | 3.42 | 11.34 | 7.91 | 4.54 | 14.30 | 12.78 | 5 |
| Fludrocortisone | 101.21 | 118.61 | 121.13 | 108.27 | 43.42 | 62.39 | 89.50 | 87.04 | 5.45 | 12.24 | 2.91 | 8.31 | 3.28 | 11.22 | 13.68 | 17.81 | 50 |
| acetate | | | | | | | | | | | | | | | | | |
| Flumetasone | 82.19 | 123.14 | 125.12 | 109.72 | 80.42 | 101.30 | 65.75 | 73.52 | 6.85 | 15.28 | 4.27 | 6.17 | 9.43 | 6.44 | 8.47 | 9.46 | 50 |
| pivalate | | | | | | | | | | | | | | | | | |
| Flumethasone | 101.50 | 77.98 | 124.79 | 121.29 | 127.98 | 126.82 | 90.67 | 91.00 | 5.58 | 9.21 | 1.87 | 8.14 | 3.26 | 4.78 | 7.56 | 14.22 | 5 |
| Fluocinolone | 111.14 | 108.57 | 123.59 | 125.87 | 129.61 | 128.75 | 88.87 | 92.62 | 5.28 | 8.97 | 3.38 | 4.63 | 3.64 | 4.14 | 8.18 | 12.59 | 5 |
| acetonide | | | | | | | | | | | | | | | | | |
| Fluorometholone | 103.64 | 106.37 | 121.97 | 110.86 | 103.82 | 127.50 | 87.21 | 100.34 | 3.59 | 12.42 | 3.61 | 9.60 | 7.85 | 4.54 | 14.32 | 11.56 | 5 |
| Flurandrenolide | 112.40 | 118.69 | 124.71 | 124.41 | 109.74 | 123.83 | 98.33 | 89.96 | 4.90 | 12.30 | 2.38 | 4.22 | 4.05 | 5.24 | 4.67 | 3.56 | 50 |
| Fluticasone | 105.95 | 68.87 | 128.81 | 90.55 | 82.62 | 122.51 | 85.04 | 89.19 | 16.51 | 5.94 | 5.04 | 6.68 | 7.35 | 5.64 | 6.31 | 6.15 | 50 |
| propionate | | | | | | | | | | | | | | | | | |
| Nandrolone | 91.46 | 106.35 | 122.04 | 86.40 | 43.36 | 121.22 | 84.24 | 82.22 | 6.36 | 10.36 | 8.41 | 6.89 | 3.91 | 4.47 | 8.61 | 7.49 | 50 |
| propionate | | | | | | | | | | | | | | | | | |
| Halcinonide | 86.90 | 103.46 | 124.03 | 93.66 | 112.06 | 121.88 | 87.18 | 85.26 | 13.56 | 13.18 | 6.71 | 4.72 | 11.61 | 3.35 | 9.54 | 7.89 | 50 |
| Dihydrodiethylstilbestrol | 113.62 | 115.08 | 115.87 | 103.69 | 103.70 | 125.15 | 86.91 | 78.62 | 13.23 | 6.38 | 3.80 | 7.83 | 4.25 | 4.34 | 11.90 | 17.91 | 1 |
| Medroxyprogesterone | 96.83 | 111.83 | 126.78 | 106.63 | 89.54 | 112.74 | 99.20 | 97.86 | 17.30 | 6.93 | 6.26 | 9.69 | 7.53 | 4.69 | 17.82 | 12.09 | 10 |
| acetate | | | | | | | | | | | | | | | | | |
| Megestrol acetate | 77.98 | 112.02 | 129.20 | 112.16 | 96.32 | 112.30 | 87.06 | 86.59 | 14.53 | 12.51 | 5.92 | 13.58 | 7.71 | 4.51 | 18.55 | 9.82 | 5 |
| Melengestrol acetate | 78.71 | 114.69 | 116.75 | 103.58 | 87.61 | 129.43 | 92.61 | 98.40 | 7.75 | 10.02 | 6.99 | 13.50 | 8.03 | 4.38 | 3.82 | 7.62 | 10 |
| Methandienone | 122.40 | 94.59 | 120.52 | 110.69 | 110.81 | 120.69 | 85.07 | 84.03 | 10.01 | 6.83 | 3.38 | 3.83 | 2.37 | 4.19 | 6.86 | 6.72 | 5 |
| Methylprednisolone | 86.06 | 121.73 | 115.98 | 109.74 | 102.62 | 121.62 | 90.30 | 95.71 | 4.30 | 11.72 | 1.67 | 3.19 | 1.57 | 3.60 | 5.55 | 9.52 | 5 |
| Norethindrone | 93.24 | 109.40 | 113.00 | 106.57 | 103.29 | 120.79 | 86.39 | 87.89 | 7.26 | 5.69 | 9.82 | 6.38 | 2.42 | 4.67 | 8.82 | 12.02 | 5 |
| Nandrolone | 101.54 | 106.17 | 120.39 | 111.03 | 111.57 | 121.59 | 83.98 | 86.25 | 3.06 | 7.87 | 5.42 | 5.85 | 4.12 | 4.84 | 9.31 | 10.96 | 5 |
| propionate | | | | | | | | | | | | | | | | | |
| Levonorgestrel | 94.79 | 115.46 | 119.71 | 107.92 | 108.16 | 113.16 | 95.42 | 87.40 | 6.10 | 8.25 | 5.41 | 11.23 | 3.48 | 4.98 | 18.04 | 9.21 | 10 |
| Prednicarbate | 92.11 | 57.61 | 114.54 | 110.81 | 99.27 | 107.83 | 99.23 | 96.62 | 3.08 | 7.31 | 3.72 | 11.55 | 10.05 | 6.24 | 17.00 | 11.52 | 10 |
| Prednisolone | 94.97 | 74.25 | 111.69 | 108.46 | 104.79 | 121.68 | 89.97 | 92.15 | 2.46 | 6.35 | 4.02 | 3.92 | 1.83 | 2.95 | 3.46 | 6.51 | 5 |
| Prednisone | 103.02 | 106.59 | 113.09 | 114.25 | 104.50 | 128.34 | 93.24 | 92.60 | 4.20 | 12.79 | 2.26 | 4.15 | 2.41 | 4.83 | 2.79 | 3.48 | 5 |
| Progesterone | 98.45 | 106.35 | 125.68 | 98.70 | 76.21 | 123.36 | 87.03 | 86.47 | 3.54 | 10.36 | 4.87 | 4.19 | 9.82 | 3.79 | 4.12 | 9.26 | 10 |
| Dehydrotestosterone | 99.53 | 125.20 | 120.52 | 110.97 | 103.13 | 110.17 | 88.64 | 93.53 | 4.16 | 6.69 | 2.68 | 3.91 | 2.19 | 4.52 | 6.76 | 6.34 | 1 |
| Testosterone | 98.74 | 113.10 | 122.23 | 103.35 | 105.15 | 105.10 | 84.63 | 88.13 | 8.85 | 7.57 | 3.81 | 5.65 | 3.01 | 5.44 | 11.71 | 14.76 | 5 |
| Trenbolone | 95.74 | 105.58 | 115.87 | 103.69 | 103.70 | 125.15 | 86.91 | 78.62 | 6.33 | 15.90 | 3.80 | 7.83 | 4.25 | 4.34 | 11.90 | 17.91 | 1 |
| Triamcinolone | 98.53 | 122.38 | 123.73 | 112.84 | 108.82 | 127.52 | 94.06 | 97.37 | 3.41 | 5.00 | 2.14 | 3.46 | 3.47 | 2.39 | 18.13 | 17.03 | 5 |
| acetonide | | | | | | | | | | | | | | | | | |
| Beclomethasone | 100.20 | 113.52 | 115.32 | 105.53 | 108.87 | 110.43 | 88.82 | 91.11 | 3.30 | 6.00 | 3.51 | 15.16 | 3.48 | 3.55 | 12.30 | 17.79 | 5 |
| Betamethasone | 102.16 | 98.48 | 112.97 | 101.51 | 81.40 | 120.46 | 90.60 | 91.41 | 4.58 | 14.32 | 8.38 | 3.21 | 16.05 | 2.83 | 8.47 | 15.25 | 5 |
| dipropionate | | | | | | | | | | | | | | | | | |
| Betamethasone | 98.38 | 103.88 | 126.08 | 101.14 | 99.28 | 128.17 | 88.80 | 82.19 | 7.11 | 13.07 | 7.24 | 8.23 | 5.45 | 5.84 | 6.08 | 15.73 | 5 |
| valerate | | | | | | | | | | | | | | | | | |
| Betamethasone | 99.67 | 103.54 | 117.89 | 110.12 | 108.49 | 124.95 | 87.95 | 90.08 | 3.37 | 6.87 | 2.83 | 4.61 | 1.77 | 3.43 | 9.03 | 12.72 | 5 |
| Dimetridazole | 106.15 | 118.97 | 112.51 | 120.10 | 111.14 | 98.37 | 100.23 | 67.27 | 5.83 | 2.18 | 4.46 | 7.45 | 6.03 | 18.01 | 8.28 | 12.42 | 5 |
| Ipronidazole | 107.61 | 98.10 | 128.72 | 85.37 | 123.02 | 96.74 | 100.26 | 60.49 | 6.12 | 4.66 | 3.19 | 7.23 | 9.18 | 17.20 | 8.19 | 17.14 | 5 |
| Metronidazole | 104.23 | 116.98 | 102.72 | 90.36 | 91.45 | 114.69 | 71.08 | 85.94 | 3.15 | 2.98 | 1.17 | 5.82 | 3.89 | 3.91 | 13.97 | 3.93 | 5 |
| Omidazole | 115.05 | 108.52 | 124.84 | 93.49 | 101.85 | 121.46 | 92.13 | 92.53 | 4.10 | 5.59 | 1.69 | 1.88 | 1.57 | 4.35 | 5.39 | 3.98 | 5 |
| Hydroxydimetridazole | 107.73 | 114.80 | 99.40 | 114.00 | 86.51 | 111.99 | 82.02 | 83.11 | 5.23 | 3.79 | 2.77 | 2.32 | 2.11 | 3.18 | 2.98 | 3.73 | 5 |
| Ronidazole | 105.28 | 67.72 | 109.14 | 91.17 | 100.58 | 118.46 | 85.57 | 90.68 | 2.75 | 4.25 | 3.78 | 3.75 | 3.07 | 2.84 | 6.61 | 5.23 | 5 |
| Secnidazole | 109.61 | 85.96 | 113.83 | 106.63 | 99.88 | 124.34 | 74.49 | 90.94 | 3.64 | 5.11 | 2.50 | 1.84 | 1.63 | 4.69 | 8.17 | 3.89 | 5 |
| Tinidazole | 113.20 | 54.52 | 113.42 | 106.33 | 105.73 | 127.16 | 92.86 | 92.87 | 2.23 | 9.34 | 4.93 | 2.21 | 4.00 | 3.49 | 2.67 | 6.02 | 5 |
| Amantadine | 87.01 | 98.69 | 73.23 | 70.07 | 76.37 | 81.25 | 83.61 | 73.62 | 2.39 | 3.36 | 1.95 | 4.78 | 2.23 | 2.50 | 3.76 | 2.01 | 1 |
| Rimantadine | 97.01 | 93.87 | 98.35 | 88.85 | 117.24 | 102.96 | 84.80 | 79.93 | 8.97 | 8.89 | 2.06 | 1.57 | 2.08 | 3.32 | 4.57 | 2.70 | 1 |
| Clindamycin | 67.37 | 94.19 | 75.04 | 68.95 | 89.71 | 82.51 | 76.88 | 75.66 | 5.42 | 10.41 | 2.81 | 6.16 | 4.44 | 6.72 | 7.81 | 6.87 | 10 |
| Lincomycin | 79.30 | 97.95 | 108.93 | 98.33 | 102.12 | 124.20 | 85.89 | 93.37 | 6.80 | 15.56 | 2.34 | 3.54 | 2.28 | 3.51 | 5.75 | 7.61 | 10 |
| Chloramphenicol | 105.07 | 98.69 | 120.22 | 125.63 | 94.37 | 127.62 | 100.56 | 102.31 | 2.86 | 3.36 | 1.71 | 14.95 | 2.52 | 4.79 | 5.69 | 1.91 | 1 |
| Florfenicol | 107.91 | 101.24 | 125.32 | 128.64 | 98.51 | 122.57 | 100.68 | 104.23 | 2.74 | 12.11 | 1.90 | 14.68 | 4.43 | 5.49 | 4.78 | 1.77 | 1 |
| Thiamphenicol | 104.32 | 98.76 | 118.80 | 109.24 | 96.44 | 113.45 | 98.65 | 102.52 | 2.99 | 9.76 | 8.75 | 17.50 | 3.64 | 3.13 | 7.36 | 2.24 | 1 |
| Ceftiofur | 76.55 | 106.17 | 81.99 | 77.33 | 48.18 | 94.09 | 88.19 | 84.90 | 4.69 | 9.41 | 3.97 | 4.30 | 6.19 | 3.70 | 8.96 | 7.26 | 10 |
| Flucioxacillin | 93.84 | 49.40 | 119.42 | 95.30 | 101.70 | 110.39 | 115.87 | 119.21 | 12.87 | 4.08 | 15.40 | 18.62 | 10.67 | 15.87 | 10.25 | 8.25 | 50 |
| Cloxacillin | 83.85 | 86.49 | 125.40 | 120.41 | 101.21 | 123.95 | 120.38 | 94.88 | 11.63 | 3.52 | 2.48 | 5.97 | 15.88 | 15.79 | 17.40 | 5.33 | 50 |
| Nafcillin | 99.68 | 121.79 | 128.20 | 117.70 | 114.65 | 116.76 | 129.63 | 104.31 | 5.01 | 5.15 | 5.15 | 6.26 | 19.08 | 8.93 | 18.41 | 19.99 | 50 |
| Piperacillin | 71.43 | 81.86 | 91.00 | 77.78 | 60.61 | 94.75 | 117.89 | 72.11 | 7.98 | 8.41 | 4.01 | 3.59 | 5.69 | 12.68 | 11.72 | 14.31 | 10 |
| Penicillin G | 53.89 | 73.48 | 100.46 | 78.30 | 63.58 | 90.22 | 120.26 | 108.04 | 16.44 | 4.68 | 6.70 | 7.10 | 11.79 | 13.18 | 12.04 | 9.51 | 10 |
| Quinocetone | 115.85 | 101.56 | 78.22 | 73.76 | 100.85 | 88.33 | 114.24 | 104.38 | 19.66 | 5.76 | 6.40 | 5.76 | 16.03 | 5.18 | 3.25 | 7.30 | 5 |
| Phenacetin | 114.22 | 89.19 | 120.73 | 113.03 | 104.63 | 129.27 | 89.58 | 86.65 | 5.03 | 9.84 | 2.31 | 2.11 | 2.37 | 4.38 | 8.06 | 11.04 | 5 |
| Salicylic acid | 62.71 | 73.48 | 62.45 | 72.77 | 54.93 | 88.88 | 50.93 | 54.51 | 6.19 | 4.68 | 2.06 | 16.06 | 7.40 | 6.42 | 7.56 | 4.51 | 10 |
| Amidopyrine | 83.29 | 92.69 | 105.75 | 126.45 | 89.33 | 110.86 | 72.24 | 125.36 | 10.21 | 16.70 | 2.48 | 9.58 | 19.60 | 10.22 | 4.21 | 5.52 | 10 |
| Antipyrine | 98.70 | 101.41 | 112.06 | 82.41 | 100.09 | 120.87 | 89.90 | 86.43 | 2.25 | 8.08 | 2.69 | 7.83 | 3.65 | 4.97 | 12.68 | 13.31 | 1 |
| Dapsone | 108.33 | 102.99 | 108.10 | 125.99 | 103.05 | 125.01 | 53.86 | 125.03 | 3.40 | 12.06 | 3.06 | 7.35 | 4.41 | 16.69 | 3.68 | 20.36 | 1 |
| Flunixin meglumine | 69.12 | 97.91 | 102.21 | 95.29 | 92.31 | 119.65 | 85.39 | 77.41 | 8.95 | 10.68 | 2.92 | 7.41 | 6.23 | 3.82 | 7.47 | 15.00 | 10 |
| Acetaminophen | 78.14 | 63.29 | 100.93 | 113.26 | 86.95 | 107.26 | 99.67 | 93.69 | 5.97 | 10.02 | 3.02 | 4.88 | 5.44 | 14.77 | 4.29 | 4.50 | 10 |
| Haloperidol | 128.37 | 96.68 | 122.37 | 105.46 | 129.29 | 123.64 | 94.14 | 84.93 | 5.95 | 12.11 | 3.01 | 4.06 | 4.73 | 4.80 | 7.84 | 10.60 | 5 |
| Imipramine | 123.20 | 98.01 | 122.38 | 110.75 | 128.27 | 129.29 | 94.11 | 87.12 | 4.06 | 17.24 | 3.12 | 3.27 | 4.78 | 6.20 | 5.18 | 5.67 | 1 |
| Nitrazepam | 122.31 | 77.98 | 120.04 | 109.52 | 106.64 | 127.43 | 89.72 | 84.32 | 3.49 | 6.84 | 1.73 | 3.14 | 4.19 | 4.26 | 6.88 | 14.93 | 5 |
| Oxazepam | 126.69 | 96.59 | 111.83 | 103.54 | 96.54 | 125.59 | 92.64 | 86.74 | 8.30 | 4.34 | 1.25 | 3.80 | 3.55 | 4.19 | 6.37 | 7.98 | 1 |
| Pemoline | 100.27 | 98.50 | 101.59 | 98.66 | 93.22 | 112.07 | 90.27 | 91.51 | 8.98 | 12.64 | 1.17 | 1.32 | 2.23 | 3.17 | 7.73 | 7.51 | 1 |
| Perphenazine | 123.23 | 107.94 | 123.31 | 95.22 | 40.77 | 108.19 | 71.73 | 68.97 | 10.84 | 7.23 | 15.11 | 14.95 | 16.37 | 5.39 | 11.82 | 9.60 | 5 |
| Promethazine | 127.29 | 115.21 | 118.33 | 125.59 | 81.62 | 111.89 | 85.47 | 85.00 | 7.92 | 4.55 | 3.80 | 8.58 | 12.12 | 12.06 | 4.67 | 5.31 | 1 |
| Propionylpromazine | 126.01 | 110.05 | 123.25 | 119.12 | 74.20 | 118.90 | 85.16 | 82.00 | 9.00 | 3.10 | 5.82 | 8.83 | 13.65 | 10.94 | 9.12 | 10.69 | 5 |
| hydrochloride | | | | | | | | | | | | | | | | | |
| Sulpiride | 74.14 | 83.94 | 82.60 | 78.33 | 71.51 | 94.84 | 72.40 | 66.27 | 6.34 | 7.04 | 1.95 | 2.58 | 8.39 | 5.74 | 3.19 | 4.54 | 5 |
| Xylazine | 88.54 | 110.65 | 110.00 | 104.87 | 107.24 | 119.56 | 87.93 | 86.58 | 6.12 | 6.20 | 2.13 | 5.92 | 3.51 | 5.67 | 5.12 | 3.19 | 1 |
| hydrochloride | | | | | | | | | | | | | | | | | |
| Acepromazin | 129.97 | 117.96 | 117.39 | 113.32 | 97.75 | 114.26 | 88.10 | 84.10 | 9.83 | 6.09 | 3.50 | 8.23 | 9.51 | 10.94 | 4.85 | 5.44 | 1 |
| Anisopirol | 107.69 | 109.21 | 115.45 | 114.37 | 115.76 | 124.77 | 91.04 | 90.25 | 3.01 | 9.58 | 1.43 | 1.86 | 4.49 | 2.87 | 4.78 | 2.62 | 1 |
| Azaperone | 115.67 | 113.48 | 114.64 | 120.76 | 95.86 | 128.45 | 92.21 | 88.55 | 4.04 | 2.57 | 2.53 | 7.75 | 9.96 | 4.14 | 6.09 | 13.20 | 1 |
| Carbamazepine | 105.06 | 93.61 | 117.25 | 110.90 | 99.85 | 124.52 | 97.11 | 94.91 | 3.72 | 6.60 | 2.06 | 1.66 | 4.88 | 5.03 | 3.53 | 2.92 | 1 |
| Chloropromazine | 107.39 | 116.52 | 122.19 | 126.84 | 58.14 | 118.44 | 78.54 | 84.75 | 3.72 | 5.87 | 7.22 | 15.57 | 16.76 | 11.96 | 13.89 | 15.28 | 5 |
| Diazepam | 49.43 | 111.03 | 119.16 | 92.36 | 104.65 | 128.39 | 86.19 | 77.60 | 7.52 | 5.15 | 1.89 | 4.09 | 4.93 | 4.91 | 6.22 | 7.04 | 1 |
| Diphenhydramine | 117.74 | 104.51 | 118.48 | 98.96 | 99.93 | 125.42 | 94.06 | 93.05 | 5.58 | 10.28 | 2.34 | 4.12 | 5.44 | 3.99 | 3.89 | 3.68 | 1 |
| hydrochloride | | | | | | | | | | | | | | | | | |
| Droperidol | 110.74 | 94.32 | 122.66 | 108.16 | 90.64 | 117.54 | 91.37 | 98.22 | 6.29 | 14.09 | 2.58 | 2.67 | 6.20 | 3.23 | 10.67 | 13.02 | 1 |
| Estazolam | 122.10 | 111.96 | 120.23 | 114.43 | 111.84 | 117.32 | 90.90 | 98.08 | 13.01 | 7.81 | 1.77 | 3.03 | 7.92 | 5.05 | 5.46 | 4.31 | 1 |
| Clarithromycin | 106.86 | 112.43 | 129.64 | 118.34 | 127.93 | 115.09 | 87.99 | 91.35 | 3.47 | 3.84 | 3.05 | 1.72 | 2.64 | 4.68 | 2.03 | 3.99 | 5 |
| Tiamulin | 107.33 | 112.22 | 125.96 | 116.60 | 102.73 | 111.81 | 100.79 | 97.17 | 1.92 | 3.88 | 2.95 | 1.97 | 2.44 | 5.00 | 4.85 | 5.27 | 5 |
| Erythromycin | 113.64 | 114.05 | 92.70 | 65.03 | 92.71 | 116.61 | 112.98 | 128.89 | 3.31 | 7.07 | 14.02 | 10.02 | 17.18 | 14.09 | 10.29 | 10.01 | 10 |
| Kitasamycin | 84.71 | 106.40 | 99.79 | 124.59 | 55.11 | 71.88 | 114.25 | 118.65 | 13.51 | 19.16 | 6.05 | 17.39 | 18.86 | 13.45 | 9.27 | 11.89 | 50 |
| Tilmicosin | 97.96 | 103.30 | 124.25 | 111.09 | 120.80 | 118.81 | 115.84 | 91.30 | 5.41 | 13.35 | 6.08 | 1.97 | 3.29 | 3.78 | 4.72 | 6.38 | 50 |
| Tylosin | 92.93 | 71.74 | 95.77 | 105.44 | 63.99 | 76.42 | 118.55 | 111.52 | 2.12 | 7.85 | 3.41 | 17.34 | 11.20 | 17.25 | 15.87 | 12.86 | 10 |
| Toltrazuril sulfoxide | 109.25 | 114.05 | 127.16 | 106.61 | 100.72 | 121.17 | 104.56 | 99.62 | 4.92 | 7.07 | 4.17 | 11.17 | 3.21 | 2.47 | 8.65 | 11.35 | 10 |
| Clopidol | 90.14 | 95.62 | 97.19 | 93.05 | 86.74 | 109.32 | 92.33 | 84.57 | 5.05 | 7.08 | 1.14 | 1.66 | 2.17 | 3.94 | 3.20 | 1.66 | 1 |
| Dinitolmide | 103.31 | 71.74 | 116.97 | 123.69 | 97.06 | 124.53 | 104.41 | 96.40 | 4.26 | 7.85 | 1.35 | 12.60 | 2.94 | 4.29 | 4.86 | 1.07 | 10 |
| Halofuginone | 70.44 | 77.60 | 88.60 | 90.78 | 103.34 | 100.83 | 85.63 | 81.77 | 3.75 | 10.19 | 4.02 | 7.05 | 5.96 | 6.44 | 7.38 | 7.99 | 10 |
| Levamisole | 92.81 | 106.75 | 103.76 | 97.12 | 96.85 | 116.27 | 82.84 | 85.35 | 5.75 | 9.77 | 1.32 | 2.28 | 1.74 | 4.22 | 2.12 | 2.01 | 5 |
| Buprofezin | 69.15 | 111.58 | 117.74 | 104.67 | 69.20 | 115.68 | 95.71 | 89.23 | 16.80 | 16.74 | 4.57 | 3.66 | 8.66 | 2.40 | 6.12 | 9.16 | 5 |
| Carboxin | 128.42 | 114.79 | 120.52 | 126.03 | 101.03 | 115.15 | 94.17 | 76.45 | 11.56 | 5.57 | 2.41 | 2.90 | 14.33 | 15.95 | 5.93 | 7.06 | 10 |
| 3-Hydroxycarbofuran | 102.23 | 121.30 | 121.18 | 107.42 | 58.32 | 129.54 | 104.91 | 96.44 | 2.27 | 8.90 | 4.14 | 5.09 | 15.73 | 2.95 | 5.15 | 3.10 | 10 |
| Clothianidin | 100.67 | 102.14 | 122.77 | 114.71 | 97.64 | 113.95 | 106.26 | 96.72 | 5.84 | 11.58 | 3.62 | 2.90 | 1.99 | 3.89 | 4.40 | 3.64 | 10 |
| Cyromazine | 50.87 | 47.96 | 46.67 | 75.40 | 46.51 | 45.03 | 59.53 | 57.28 | 8.00 | 3.11 | 2.66 | 1.98 | 3.06 | 4.19 | 1.06 | 2.15 | 10 |
| Diuron | 123.12 | 114.05 | 123.49 | 113.10 | 101.83 | 128.10 | 95.31 | 85.99 | 7.51 | 4.26 | 2.53 | 9.75 | 1.60 | 3.22 | 17.47 | 9.86 | 10 |
| Ethopabate | 97.07 | 113.89 | 120.67 | 111.75 | 107.17 | 123.01 | 90.18 | 93.62 | 7.13 | 5.21 | 2.94 | 4.68 | 13.92 | 3.14 | 4.92 | 6.49 | 5 |
| Fipronil | 107.91 | 60.46 | 119.15 | 116.44 | 89.89 | 119.16 | 102.74 | 104.60 | 5.21 | 4.41 | 17.92 | 16.36 | 6.10 | 8.26 | 6.45 | 10.46 | 10 |
| Fluridone | 98.62 | 107.17 | 124.11 | 108.42 | 70.59 | 122.24 | 101.86 | 93.49 | 3.12 | 8.43 | 2.83 | 7.88 | 3.03 | 4.16 | 15.13 | 19.52 | 10 |
| Acephate | 69.28 | 113.70 | 82.98 | 82.76 | 105.45 | 79.01 | 81.65 | 75.20 | 6.80 | 4.00 | 4.10 | 2.65 | 2.06 | 4.24 | 2.89 | 4.21 | 10 |
| Hexazinone | 101.50 | 114.90 | 122.89 | 121.84 | 121.35 | 118.91 | 100.72 | 97.85 | 2.31 | 5.52 | 2.06 | 4.32 | 2.94 | 1.13 | 2.23 | 3.12 | 10 |
| Imazalil | 95.33 | 119.23 | 127.32 | 128.45 | 104.38 | 118.91 | 102.39 | 90.93 | 2.66 | 17.27 | 3.59 | 2.77 | 1.03 | 2.54 | 3.22 | 8.71 | 10 |
| Imidacloprid | 98.65 | 78.83 | 122.85 | 118.74 | 93.96 | 121.01 | 107.34 | 95.91 | 4.29 | 9.83 | 2.19 | 3.54 | 2.30 | 2.02 | 3.62 | 4.54 | 10 |
| Linuron | 100.08 | 113.24 | 123.08 | 111.60 | 103.58 | 127.59 | 90.49 | 74.90 | 3.67 | 9.03 | 3.77 | 12.30 | 1.57 | 3.79 | 11.24 | 8.26 | 10 |
| Metribuzin | 102.48 | 92.83 | 126.03 | 115.57 | 97.33 | 124.88 | 93.37 | 85.85 | 2.82 | 9.89 | 2.71 | 2.60 | 5.81 | 2.14 | 14.10 | 12.87 | 5 |
| Myclobutanil, | 100.72 | 109.80 | 126.18 | 121.11 | 101.49 | 126.01 | 91.26 | 97.30 | 2.87 | 3.85 | 2.42 | 4.26 | 3.58 | 2.15 | 7.16 | 11.01 | 5 |
| Acetamiprid | 97.83 | 114.85 | 123.57 | 84.65 | 107.39 | 123.45 | 98.57 | 94.28 | 3.46 | 4.71 | 1.82 | 8.73 | 3.65 | 2.31 | 1.80 | 4.06 | 10 |
| Norflurazon | 98.95 | 79.67 | 127.52 | 117.07 | 68.71 | 125.56 | 103.14 | 96.69 | 5.56 | 9.43 | 2.12 | 3.08 | 10.19 | 1.81 | 7.91 | 8.44 | 10 |
| Propyzamide | 98.87 | 119.15 | 127.33 | 113.55 | 99.07 | 124.81 | 96.22 | 71.15 | 4.55 | 5.75 | 1.84 | 8.58 | 3.42 | 3.32 | 16.02 | 19.06 | 50 |
| Atrazine | 88.80 | 95.51 | 129.21 | 116.81 | 102.43 | 128.46 | 89.12 | 77.37 | 8.02 | 15.36 | 1.41 | 2.65 | 1.89 | 1.53 | 3.01 | 8.89 | 5 |
| Simazine | 54.74 | 61.79 | 122.97 | 120.34 | 102.81 | 126.71 | 90.33 | 76.06 | 10.80 | 7.79 | 2.61 | 2.99 | 1.36 | 2.08 | 4.15 | 11.37 | 5 |
| Desethyl atrazine | 99.77 | 67.22 | 125.26 | 127.57 | 121.25 | 120.78 | 101.41 | 87.52 | 2.82 | 6.60 | 1.96 | 2.45 | 6.06 | 2.55 | 3.06 | 2.68 | 10 |
| Azoxystrobin | 103.10 | 83.63 | 129.88 | 118.44 | 112.11 | 123.62 | 91.49 | 90.10 | 2.01 | 16.42 | 1.34 | 3.59 | 9.16 | 8.92 | 5.70 | 8.55 | 5 |
| Benoxacor | 105.02 | 94.24 | 111.70 | 105.70 | 98.43 | 122.59 | 92.47 | 46.93 | 5.01 | 5.43 | 8.08 | 11.16 | 12.85 | 7.86 | 9.40 | 19.82 | 50 |
| Scopolamine | 99.57 | 76.10 | 120.33 | 90.26 | 70.95 | 76.19 | 66.52 | 69.30 | 1.78 | 7.82 | 5.62 | 1.60 | 3.18 | 1.75 | 2.31 | 2.36 | 5 |
| Valnemulin | 101.89 | 83.79 | 116.39 | 122.77 | 96.23 | 119.38 | 83.76 | 92.29 | 2.17 | 6.10 | 2.45 | 4.94 | 3.76 | 4.60 | 9.08 | 14.38 | 5 |
| Atropine | 99.74 | 76.60 | 120.95 | 89.87 | 94.80 | 87.52 | 75.00 | 73.48 | 1.93 | 9.18 | 1.97 | 2.18 | 7.29 | 1.55 | 6.35 | 20.00 | 1 |

9.3 Matrix Effect

The matrix effect of the sample was evaluated by comparing the standard solution and the spiked sample liquid before the nitrogen blowing in the chromatographic peak area. The results showed that the substances in the matrix may show matrix inhibition or matrix enhancement, where a matrix effect value between 0.85 and 1.15 indicated that there was no significant matrix effect; a matrix effect value greater than 1.15 indicated matrix enhancement; and a matrix effect value less than 0.85 indicated matrix inhibition. The matrix effect values measured herein ranged from 0.11 to 8.03, therefore, it was required to plot a matrix-matched standard curve for the quantification.

Described above are merely preferred embodiments of the invention. It should be noted that any modification and change made by those skilled in the art without departing from the spirit of the invention should fall within the scope of the invention.

What is claimed is:

1. A screening and confirmation method for veterinary drugs and additives in animal-derived food, comprising:
(1) dissolving 10 mg of each reference standard of the veterinary drugs and additives followed by diluting to 10 mL in a volumetric flask to prepare a 1 mg/mL stock solution of each reference standard;
wherein the veterinary drugs and additives are selected from:
(a) sulfonamides: sulfamethazine, sulformethoxine, sulfisoxazole, sulfaguanidine, sulfamerazine, sulfamethylthiadizaole, sulfamethoxazole, sulfamethoxydiazine, sulfamethoxypyridazine, sulfamonomethoxine, sulfamethazole, sulfapyridinee, sulfaquinoxaline, sulfathiazole, sulfisomidine, trimethoprim, diaveridine, phthalylsulfathiazole, sulfabenzamide, sulfacetamide, sulfachlorpyridazine, sulfachloropyrazine, sulfadiazine and sulfadimethoxine;
(b) fluoroquinolones: lomefloxacin, marbofloxacin, nadifloxacin, nalidixic acid, norfloxacin, ofloxacin, orbifloxacin, oxolinic acid, pipemidic acid, pefloxacin, cinoxacin, sarafloxacin, sparfloxacin, ciprofloxacin, danofloxacin, difloxacin, enoxacin, enrofloxacin, fleroxacin, flumequine and gatifloxacin;
(c) benzimidazoles: mebendazole, mebendazole-amine, 2-aminofluorobenzimidazole, oxfendazole, oxibendazole, parbendazole, 5-hydroxythiabendazole, tiabendazole, albendazole, albendazole sulfone, albendazole sulfoxide, amino albendazole sulfone, benzimidazole, febantel and lobendazole;
(d) stimulants: cyproheptadine, fenoterol, labetalol, penbutolol, phenylethanolamine A, ractopamine, salbutamol, salmeterol, terbutaline, mabuterol, tulobuterol, bambuterol, brombuterol, cimaterol, cimbuterol, clenbuterol, clenproperol and clonidine;
(e) hormones: chlormadinone acetate, 17a-methyltestosterone, clobetasol propionate, clobetasone butyrate, corticosterone, hydrocortisone, cortisone acetate, cortisone, deflazacort, dexamethasone, diflorasone diacetate, epitestosterone, estradiol benzoate, fludrocortisone acetate, flumetasone pivalate, flumethasone, fluocinolone acetonide, fluorometholone, flurandrenolide, fluticasone propionate, nandrolone propionate, halcinonide, dihydrodiethylstilbestrol, medroxyprogesterone acetate, megestrol acetate, melengestrol acetate, methandienone, methylprednisolone, norethindrone, nandrolone propionate, levonorgestrel, prednicarbate, prednisolone, prednisone, progesterone, dehydrotestosterone, testosterone, trenbolone, triamcinolone acetonide, beclomethasone, betamethasone dipropionate, betamethasone valerate and betamethasone;
(f) nitroimidazoles: dimetridazole, ipronidazole, metronidazole, ornidazole, hydroxydimetridazole, ronidazole, secnidazole and tinidazole;
(g) antiviral drugs: amantadine and rimantadine;
(h) lincosamide: clindamycin and lincomycin;
(i) amphenicols: chloramphenicol, florfenicol and thiamphenicol;
(j) penicillins: ceftiofur, flucloxacillin, cloxacillin, nafcillin, piperacillin and penicillin G;
(k) quinoxalines: quinocetone;
(l) antipyretics and analgesics: phenacetin, salicylic acid, amidopyrine, antipyrine, dapsone, flunixin meglumine and acetaminophen;
(m) psychoactive drugs: haloperidol, imipramine, nitrazepam, oxazepam, pemoline, perphenazine, promethazine, propionylpromazine hydrochloride, sulpiride, xylazine, acepromazin, anisopirol, azaperone, carbamazepine, chloropromazine, diazepam, diphenhydramine, droperidol, estazolam, clarithromycin, tiamulin, erythromycin, kitasamycin, tilmicosin and tylosin;
(n) anthelmintics: toltrazuril sulfoxide, clopidol, dinitolmide, halofuginone and levamisole;
(o) pesticides: buprofezin, carboxin, 3-hydroxycarbofuran, clothianidin, cyromazine, diuron, ethopabate, fipronil, fluridone, acephate, hexazinone, imazalil, imidacloprid, linuron, metribuzin, myclobutanil, acetamiprid, norflurazon, propyzamide, atrazine, simazine, desethyl atrazine, azoxystrobin and benoxacor;
(p) other substances: scopolamine, valnemulin and atropine;
the norfloxacin is dissolved with water and diluted with methanol to 10 mL; each of the pesticides is dissolved with acetone and then diluted with methanol to 10 mL; each of the diaveridine, albendazole, albendazole sulfone and febantel is dissolved with formic acid and diluted with methanol to 10 mL; and the rest substances are dissolved with a mixed solvent of methanol and ammonia water or a mixed solvent of methanol and dimethyl sulfoxide, and then diluted with methanol to 10 mL;
(2) pipetting 100 μL of the stock solution of each reference standard in the same category in Table 1 to a volumetric flask followed by diluting with methanol to 10 mL to accordingly prepare 16 first mixed standard solutions, wherein a concentration of each reference standard in the corresponding first mixed standard solution thereof is 10 μg/mL; pipetting 100 μL of each first mixed standard solution to another volumetric flask followed by diluting with methanol to 10 mL to prepare a second mixed standard solution, wherein each reference standard in the second mixed standard solution has a concentration of 100 ng/mL; and pipetting 100 μL, 500 μL, 1 mL and 5 mL, respectively, to a volumetric flask followed by diluting with a 20% aqueous methanol solution to 10 mL to prepare a series of mixed standard working solutions, wherein each reference standard in the series of mixed standard working solutions has a concentration of 1 ng/mL, 5 ng/mL, 10 ng/mL and 50 ng/mL, respectively;
(3) adding 5 g of a sample to a 50 mL centrifuge tube; adding an extracting solution followed by vortex mixing; shaking and centrifuging the centrifuge tube; transferring a first supernatant to another 50 mL centrifuge tube;
if the sample is muscle or poultry egg, treating a precipitate with the extracting solution again to collect an extract; combining the first supernatant with the extract to produce a mixture; adding 6 g of anhydrous sodium sulfate and 1.5 g of sodium chloride to the mixture followed by vortex mixing for 30 s and standing; and centrifuging the another 50 mL centrifuge tube to produce a second supernatant;
if the sample is cow's milk or goat's milk, adding 4 g of anhydrous sodium sulfate and 1 g of sodium chloride to the first supernatant followed by vortex mixing for 30 s and standing; and centrifuging the another 50 mL centrifuge tube to produce a second supernatant;
(4) activating and equilibrating a solid phase extraction column sequentially with 4 mL of methanol and 4 mL of water; rinsing the solid phase extraction column with 2 mL of the second supernatant and discarding a first effluent; loading 5 mL of the second supernatant to the solid phase extraction column and collecting a second effluent with a centrifuge tube; drying 4 mL of the second effluent in nitrogen flow at 40° C.; adding 200 μL of methanol to the dried product followed by vortex mixing for 10 s; adding 800 μL of pure water followed by vortex mixing for 30 s to produce a sample solution; and filtering the sample solution with 0.22 m nylon filter membrane and collecting a filtrate for use;
(5) diluting each of the first mixed standard solutions such that each reference standard in the corresponding diluted first mixed standard solution thereof has a concentration of 1 μg/mL; subjecting each of the diluted first mixed standard solutions to full scan and secondary scan in positive and negative modes using a ultra high performance liquid chromatography-quadrupole/electrostatic field orbitrap high resolution mass spectrometer; analyzing the scan results to obtain information of each reference standard through Trace Finder software of Q-Exactive plus; and establishing a mass spectrometry database based on the information of each reference standard; wherein the information of each reference standard comprises retention time, precise molecular weight of parent ion, addition mode, fragment ions, isotope abundance ratio and a fragment ion mass spectrometry generated through the superposition of fragment ion mass spectrometry obtained at collision energies of 20 eV, 40 eV and 60 eV; the isomeride are further identified through the analysis of single standard reference; and the mass spectrometry database is shown in Table 1;
TABLE 1
Mass spectrometry library information

| | | | | | | | Retention |
|---|---|---|---|---|---|---|---|
| | | Molecular | Addition | Parent | | | time |
| Category | Compound | formula | mode | ion | Fragment 1 | Fragment 2 | (min) |
| Sulfonamides | Sulfamethazine | C₁₂H₁₄N₄O₂S | M + H | 279.09102 | 156.01138 | 124.08694 | 8.44 |
| | Sulformethoxine | C₁₂H₁₄N₄O₄S | M + H | 311.08085 | 156.01138 | 140.04545 | 9.55 |
| | Sulfisoxazole | C₁₁H₁₃N₃O₃S | M + H | 268.07504 | 156.0111 | 108.04462 | 9.83 |
| | Sulfaguanidine | C₇H₁₀N₄O₂S | M + H | 215.05972 | 156.01138 | 108.04439 | 3.43 |
| | Sulfamerazine | C₁₁H₁₂N₄O₂S | M + H | 265.07537 | 190.02809 | 156.01138 | 7.89 |
| | Sulfamethylthiadizaole | C₉H₁₀N₄O₂S₂ | M + H | 271.03179 | 156.01138 | 108.04439 | 8.31 |
| | Sulfamethoxazole | C₁₀H₁₁N₃O₃S | M + H | 254.05939 | 156.01138 | 108.04439 | 9.57 |
| | Sulfamethoxydiazine | C₁₁H₁₂N₄O₃S | M + H | 281.07029 | 108.04465 | 156.01115 | 8.53 |
| | Sulfamethoxypyridazine | C₁₁H₁₂N₄O₃S | M + H | 281.07029 | 215.09274 | 156.01138 | 8.41 |
| | Sulfamonomethoxine | C₁₁H₁₂N₄O₃S | M + H | 281.07029 | 215.09274 | 156.01138 | 8.9 |
| | Sulfamethazole | C₁₁H₁₃N₃O₃S | M + H | 268.07504 | 156.0111 | 108.04462 | 8.03 |
| | Sulfapyridinee | C₁₁H₁₁N₃O₂S | M + H | 250.06447 | 184.08692 | 156.01138 | 7.55 |
| | Sulfaquinoxaline | C₁₄H₁₂N₄O₂S | M + H | 301.07537 | 156.01138 | 108.04439 | 10.39 |
| | Sulfathiazole | C₉H₉N₃O₂S₂ | M + H | 256.02089 | 156.01138 | 108.04439 | 7.28 |
| | Sulfisomidine | C₁₂H₁₄N₄O₂S | M + H | 279.09102 | 124.08692 | 108.04439 | 6.57 |
| | Trimethoprim | C₁₄H₁SN₄O₃ | M + H | 291.14517 | 275.11387 | 261.09822 | 7.28 |
| | Diaveridine | C₁₃H₁₆N₄O₂ | M + H | 261.1346 | 245.1033 | 123.06652 | 7 |
| | Phthalylsulfathiazole | C₁₇H₁₃N₃O₅S₂ | M + H | 404.03694 | 149.02313 | 256.02032 | 9.07 |
| | Sulfabenzamide | C₁₃H₁₂N₂O₃S | M + H | 277.06414 | 156.01138 | 108.04439 | 10.32 |
| | Sulfacetamide | C₈H₁₀N₂O₃S | M + H | 215.04849 | 173.03792 | 156.01138 | 6.73 |
| | Sulfachlorpyridazine | C₁₀H₉ClN₄O₂S | M + H | 285.02075 | 156.01138 | 108.04439 | 9.25 |
| | Sulfachloropyrazine | C₁₀H₉ClN₄O₂S | M + H | 285.02075 | 130.0166 | 156.01111 | 10.35 |
| | Sulfadiazine | C₁₀H₁₀N₄O₂S | M + H | 251.05972 | 156.01138 | 108.04439 | 7.13 |
| | Sulfadimethoxine | C₁₂H₁₄N₄O₄S | M + H | 311.08085 | 156.07675 | 108.04439 | 10.43 |
| Fluoroquinolones | Lomefloxacin | C₁₇H₁₉F₂N₃O₃ | M + H | 352.14672 | 308.1569 | 265.1147 | 7.58 |
| | Marbofloxacin | C₁₇H₁₉FN₄O₄ | M + H | 363.14631 | 320.10411 | 72.08078 | 7.17 |
| | Nadifloxacin | C₁₉H₂₁FN₂O₄ | M + H | 361.15581 | 343.14389 | 283.08725 | 10.76 |
| | Nalidixic acid | C₁₂H₁₂N₂O₃ | M + H | 233.09207 | 205.06077 | 187.0502 | 11.43 |
| | Norfloxacin | C₁₆H₁₈FN₃O₃ | M + H | 320.1405 | 276.15067 | 233.10847 | 7.24 |
| | Ofloxacin | C₁₈H₂₀FN₃O₄ | M + H | 362.15106 | 318.16123 | 261.10338 | 7.31 |
| | Orbifloxacin | C₁₉H₂₀F₃N₃O₃ | M + H | 396.15295 | 352.16312 | 295.10527 | 7.89 |
| | Oxolinic acid | C₁₃H₁₁NO₅ | M + H | 262.071 | 244.06043 | 234.0397 | 9.95 |
| | Pipemidic acid | C₁₄H₁₇N₅O₃ | M + H | 304.14042 | 290.16632 | 233.10847 | 6.75 |
| | Pefloxacin | C₁₇H₂₀FN₃O₃ | M + H | 334.15615 | 217.10839 | 189.0779 | 7.35 |
| | Cinoxacin | C₁₂H₁₀N₂O₅ | M + H | 263.06625 | 235.07133 | 217.06077 | 9.47 |
| | Sarafloxacin | C₂₀H₁₇F₂N₃O₃ | M + H | 386.13107 | 342.14125 | 299.09905 | 8.16 |
| | Sparfloxacin | C₁₉H₂₂F₂N₄O₃ | M + H | 393.17327 | 349.18344 | 292.12559 | 8.22 |
| | Ciprofloxacin | C₁₇H₁₈FN₃O₃ | M + H | 332.1405 | 288.15067 | 245.10847 | 7.37 |
| | Danofloxacin | C₁₉H₂₀FN₃O₃ | M + H | 358.15615 | 314.16632 | 283.12412 | 7.54 |
| | Difloxacin | C₂₁H₁₉F₂N₃O₃ | M + H | 400.14672 | 356.15689 | 299.09905 | 8.27 |
| | Enoxacin | C₁₅H₁₇FN₄O₃ | M + H | 321.13575 | 277.14592 | 257.13969 | 7.13 |
| | Enrofloxacin | C₁₉H₂₂FN₃O₃ | M + H | 360.1718 | 316.18197 | 245.10847 | 7.73 |
| | Fleroxacin | C₁₇H₁₈F₃N₃O₃ | M + H | 370.1373 | 326.14747 | 269.08962 | 7.36 |
| | Flumequine | C₁₄H₁₂FNO₃ | M + H | 262.0874 | 244.07683 | 220.04045 | 11.59 |
| | Gatifloxacin | C₁₉H₂₂FN₃O₄ | M + H | 376.16671 | 332.17688 | 289.13468 | 7.97 |
| Stimulants | Cyproheptadine | C₂₁H₂₁N | M + H | 288.17468 | 191.08553 | 96.08078 | 10.67 |
| | Fenoterol | C₁₇H₂₁NO₄ | M + H | 304.15433 | 286.14377 | 135.08044 | 6.57 |
| | Labetalol | C₁₉H₂₄N₂O₃ | M + H | 329.18597 | 311.1754 | 294.14886 | 8.97 |
| | Penbutolol | C₁₈H₂₉NO₂ | M + H | 292.22711 | 236.16451 | 201.12739 | 11.08 |
| | Phenylethanolamine A | C₁₉H₂₄N₂O₄ | M + H | 345.18088 | 327.17032 | 150.09134 | 10.03 |
| | Ractopamine | C₁₈H₂₃NO₃ | M + H | 302.17507 | 284.16451 | 164.10699 | 7.56 |
| | Salbutamol | C₁₃H₂₁NO₃ | M + H | 240.15942 | 148.07569 | 166.08625 | 5.75 |
| | Salmeterol | C₂₅H₃₇NO₄ | M + H | 416.27954 | 398.26897 | 380.2584 | 11.03 |
| | Terbutaline | C₁₂H₁₉NO₃ | M + H | 226.14377 | 170.08117 | 152.07061 | 5.71 |
| | Mabuterol | C₁₃H₁₈ClF₃N₂O | M + H | 311.11325 | 237.03951 | 217.03343 | 9 |
| | Tulobuterol | C₁₂H₁₈ClNO | M + H | 228.11497 | 172.05237 | 154.0418 | 8.25 |
| | Bambuterol | C₁₈H₂₉N₃O₅ | M + H | 368.218 | 312.1554 | 294.14483 | 8.65 |
| | Brombuterol | C₁₂H₁₈Br₂N₂O | M + H | 364.98586 | 290.9127 | 211.99436 | 8.71 |
| | Cimaterol | C₁₂H₁₇N₃O | M + H | 220.14444 | 202.13387 | 160.08692 | 5.93 |
| | Cimbuterol | C₁₃H₁₉N₃O | M + H | 234.16009 | 160.08656 | 143.06007 | 6.61 |
| | Clenbuterol | C₁₂H₁₈Cl₂N₂O | M + H | 277.0869 | 259.07633 | 203.01373 | 8.31 |
| | Clenproperol | C₁₁H₁₆Cl₂N₂O | M + H | 263.07125 | 245.06068 | 203.01373 | 7.85 |
| | Clonidine | C₉H₉Cl₂N₃ | M + H | 230.02463 | 212.99808 | / | 6.77 |
| Hormones | Chlormadinone acetate | C₂₃H₂₉ClO₄ | M + H | 405.18271 | 309.1849 | 267.1743 | 15.67 |
| | 17a-methyltestosterone | C₂₀H₃₀O₂ | M + H | 303.23186 | 285.22129 | 267.21073 | 13.63 |
| | Clobetasol propionate | C₂₅H₃₂ClFO₅ | M + H | 467.19951 | 263.143 | 147.0804 | 15.08 |
| | Clobetasone butyrate | C₂₆H₃₂ClFO₅ | M + H | 479.19951 | 279.138 | 343.1459 | 16.13 |
| | Corticosterone | C₂₁H₃₀O₄ | M + H | 347.22169 | 329.21112 | 311.20056 | 11.61 |
| | Hydrocortisone | C₂₁H₃₀O₅ | M + H | 363.2166 | 327.19547 | 309.18491 | 10.34 |
| | Cortisone acetate | C₂₃H₃₀O₆ | M + H | 403.21152 | 343.19038 | 163.11174 | 12.53 |
| | Cortisone | C₂₁H₂₈O₅ | M + H | 361.20095 | 163.1117 | 121.0648 | 10.45 |
| | Deflazacort | C₂₅H₃₁NO₆ | M + H | 442.22241 | 142.0499 | 312.1958 | 12.39 |
| | Dexamethasone | C₂₂H₂₉FO₅ | M + H | 393.20718 | 355.19039 | 337.17982 | 11.21 |
| | Diflorasone diacetate | C₂₆H₃₂F₂O₇ | M + H | 495.21889 | 121.0648 | 317.1536 | 13.91 |
| | Epitestosterone | C₁₉H₂₈O₂ | M + H | 289.21621 | 97.0648 | 109.0648 | 13.93 |
| | Estradiol benzoate | C₂₅H₂₈O₃ | M + H | 377.21112 | 279.1743 | 321.1849 | 12.22 |
| | Fludrocortisone acetate | C₂₃H₃₁FO₆ | M + H | 423.21774 | 181.1023 | 239.143 | 12.22 |
| | Flumetasone pivalate | C₂₇H₃₆F₂O₆ | M + H | 495.25527 | 57.0699 | 121.0648 | 15.25 |
| | Flumethasone | C₂₂H₂₈F₂O₅ | M + H | 411.19776 | 235.1117 | 277.1587 | 11.2 |
| | Fluocinolone acetonide | C₂₄H₃₀F₂O₆ | M + H | 453.20832 | 121.0648 | 337.1434 | 11.86 |
| | Fluorometholone | C₂₂H₂₉FO₄ | M + H | 377.21226 | 279.1743 | 135.0804 | 12.22 |
| | Flurandrenolide | C₂₄H₃₃FO₆ | M + H | 437.23339 | 361.1798 | 105.0699 | 12.01 |
| | Fluticasone propionate | C₂₅H₃₁F₃O₅S | M + H | 501.19171 | 121.0648 | 275.143 | 15.08 |
| | Nandrolone propionate | C₂₁H₃₀O₃ | M + H | 331.22677 | 257.18999 | 239.17943 | 17.38 |
| | Halcinonide | C₂₄H₃₂ClFO₅ | M + H | 455.19951 | 121.0648 | 377.1514 | 14.98 |
| | Dihydrodiethylstilbestrol | C₁₈H₂₂O₂ | M + H | 271.16926 | 109.1117 | 253.1587 | 11.92 |
| | Medroxyprogesterone | C₂₄H₃₄O₄ | M + H | 387.25299 | 123.0804 | 327.2319 | 15.81 |
| | acetate | | | | | | |
| | Megestrol acetate | C₂₄H₃₂O₄ | M + H | 385.23734 | 267.1743 | 224.156 | 15.53 |
| | Melengestrol acetate | C₂₅H₃₂O₄ | M + H | 397.23734 | 279.1743 | 337.2162 | 15.82 |
| | Methandienone | C₂₀H₂₈O₂ | M + H | 301.21621 | 283.20564 | 149.13248 | 12.73 |
| | Methylprednisolone | C₂₂H₃₀O₅ | M + H | 375.2166 | 161.0961 | 135.0804 | 10.99 |
| | Norethindrone | C₂₀H₂₆O₂ | M + H | 299.20056 | 109.0648 | 281.19 | 13.12 |
| | Nandrolone propionate | C₁₈H₂₆O₂ | M + H | 275.20056 | 109.06499 | 239.17885 | 12.47 |
| | Levonorgestrel | C₂₁H₂₈O₂ | M + H | 313.21621 | 109.0648 | 245.19 | 14.23 |
| | Prednicarbate | C₂₇H₃₆O₈ | M + H | 489.24829 | 147.0804 | 289.1587 | 14.91 |
| | Prednisolone | C₂₁H₂₈O₅ | M + H | 361.20095 | 147.0804 | 171.0804 | 10.2 |
| | Prednisone | C₂₁H₂₆O₅ | M + H | 359.1853 | 341.17474 | 237.12739 | 10.27 |
| | Progesterone | C₂₁H₃₀O₂ | M + H | 315.23186 | 109.06479 | 97.06479 | 15.76 |
| | Dehydrotestosterone | C₁₉H₂₆O₂ | M + H | 287.20056 | 121.0647 | 135.1167 | 12.22 |
| | Testosterone | C₁₉H₂₈O₂ | M + H | 289.21621 | 109.06479 | 97.06479 | 13.03 |
| | Trenbolone | C₁₈H₂₂O₂ | M + H | 271.16926 | 253.15869 | 199.11174 | 11.92 |
| | Triamcinolone | C₂₄H₃₁FO₆ | M + H | 435.21774 | 397.201 | 339.1591 | 11.69 |
| | acetonide | | | | | | |
| | Beclomethasone | C₂₂H₂₉ClO₅ | M + H | 409.17763 | 391.1671 | 279.1743 | 11.45 |
| | Betamethasone | C₂₈H₃₇FO₇ | M + H | 505.25961 | 279.1741 | 319.1693 | 15.45 |
| | dipropionate | | | | | | |
| | Betamethasone | C₂₇H₃₇FO₆ | M + H | 477.26469 | 279.1743 | 147.0804 | 14.55 |
| | valerate | | | | | | |
| | Betamethasone | C₂₂H₂₉FO₅ | M + H | 393.20718 | 355.19039 | 279.17434 | 11.14 |
| Nitroimidazoles | Dimetridazole | C₅H₇N₃O₂ | M + H | 142.0611 | 112.06311 | 95.06037 | 7.17 |
| | Ipronidazole | C₇H₁₁N₃O₂ | M + H | 170.0924 | 140.09441 | 123.09167 | 10.33 |
| | Metronidazole | C₆H₉N₃O₃ | M + H | 172.07167 | 128.04545 | 82.05255 | 6.39 |
| | Ornidazole | C₇H₁₀ClN₃O₃ | M + H | 220.04835 | 128.04547 | 203.14304 | 8.77 |
| | Hydroxydimetridazole | C₅H₇N₃O₃ | M + H | 158.05602 | 140.0455 | 110.0475 | 6.42 |
| | Ronidazole | C₆H₈N₄O₄ | M + H | 201.06183 | 140.04545 | 110.04746 | 7.03 |
| | Secnidazole | C₇H₁₁N₃O₃ | M + H | 186.08732 | 128.0455 | 111.0427 | 7.56 |
| | Tinidazole | C₈H₁₃N₃O₄S | M + H | 248.06995 | 154.0611 | 110.08384 | 8.12 |
| Antiviral | Amantadine | C₁₀H₁₇N | M + H | 152.14338 | 135.11683 | 107.08553 | 7.48 |
| drugs | Rimantadine; | C₁₂H₂₁N | M + H | 180.17468 | 163.14778 | 81.0704 | 9 |
| Lincosamides | Clindamycin | C₁₈H₃₃ClN₂O₅S | M + H | 425.18715 | 126.12773 | / | 8.69 |
| | Lincomycin | C₁₈H₃₄N₂O₆S | M + H | 407.221 | 126.12773 | 359.21766 | 6.76 |
| Amphenicols | Chloramphenicol | C₁₁H₁₂C₁₂N₂O₅ | M − H | 321.00505 | 257.03346 | 194.04588 | 10.11 |
| | Florfenicol | C₁₂H₁₄Cl₂FNO₄S | M − H | 355.99319 | 335.98696 | 185.02779 | 9.96 |
| | Thiamphenicol | C₁₂H₁₅Cl₂NO₅S | M − H | 353.99752 | 290.02593 | 185.02779 | 8.27 |
| β-Lactams | Ceftiofur | C₁₉H₁₇N₅O₇S₃ | M + H | 524.03629 | 241.03899 | 210.02065 | 9.63 |
| | Flucloxacillin | C₁₉H₁₇ClFN₃O₅S | M + H | 454.06342 | 160.04242 | 114.03742 | 12.5 |
| | Cloxacillin | C₁₉H₁₈ClN₃O₅S | M + H | 436.07285 | 277.0374 | 206.0367 | 12.26 |
| | Nafcillin | C₂₁H₂₂N₂O₅S | M + H | 415.13222 | 199.0754 | 171.0441 | 12.54 |
| | Piperacillin | C₂₃H₂₇N₅O₇S | M + H | 518.1704 | 143.0815 | 115.0502 | 10.16 |
| | Penicillin G | C₁₆H₁₈N₂O₄S | M + H | 335.106 | 217.06414 | 176.07061 | 10.77 |
| Quinoxalines | Quinocetone | C₁₈H₁₄N₂O₃ | M + H | 307.10772 | 143.06018 | 273.10178 | 11.99 |
| Antipyretics | Phenacetin | C₁₀H₁₃NO₂ | M + H | 180.10191 | 110.0602 | 138.09109 | 10.11 |
| and analgesics | Salicylic acid | C₇H₆O₃ | M − H | 137.02442 | 93.0346 | 65.0397 | 3.74 |
| | Amidopyrine | C₁₃H₁₇N₃O | M + H | 232.14444 | 113.10738 | 98.08418 | 6.46 |
| | Antipyrine | C₁₁H₁₂N₂O | M + H | 189.10224 | 147.09135 | 161.10696 | 8.39 |
| | Dapsone | C₁₂H₁₂N₂O₂S | M + H | 249.06922 | 156.01122 | 108.04431 | 9.03 |
| | Flunixin meglumine | C₁₄H₁₁F₃N₂O₂ | M + H | 297.08454 | 279.07397 | 264.0505 | 13.66 |
| | Acetaminophen | C₈H₉NO₂ | M + H | 152.07061 | 110.06004 | / | 6.25 |
| Psychoactive | Haloperidol | C₂₁H₂₃ClFNO₂ | M + H | 376.14741 | 358.13685 | 165.07102 | 10.3 |
| drugs | Imipramine | C₁₉H₂₄N₂ | M + H | 281.20123 | 86.0963 | 236.1434 | 10.71 |
| | Nitrazepam | C₁₅H₁₁N₃O₃ | M + H | 282.08732 | 268.08383 | 207.09126 | 11.53 |
| | Oxazepam | C₁₅H₁₁ClN₂O₂ | M + H | 287.05818 | 241.05203 | 269.04703 | 11.49 |
| | Pemoline | C₉H₈N₂O₂ | M + H | 177.06585 | 106.0651 | 79.0542 | 7.74 |
| | Perphenazine | C₂₁H₂₆ClN₃OS | M + H | 404.15579 | 143.11763 | 171.14877 | 10.83 |
| | Promethazine | C₁₇H₂₀N₂S | M + H | 285.142 | 198.0372 | 86.09643 | 10.34 |
| | Propionylpromazine | C₂₀H₂₄N₂OS | M + H | 341.16821 | 86.0964 | 58.0651 | 10.95 |
| | hydrochloride | | | | | | |
| | Sulpiride | C₁₅H₂₃N₃O₄S | M + H | 342.1482 | 214.0169 | 112.1121 | 6.21 |
| | Xylazine | C₁₂H₁₆N₂S | M + H | 221.1107 | 90.0372 | 164.0528 | 8.27 |
| | hydrochloride | | | | | | |
| | Acepromazin | C₁₉H₂₂N₂OS | M + H | 327.15256 | 254.06341 | 86.09643 | 10.2 |
| | Anisopirol | C₁₉H₂₄FN₃O | M + H | 330.19762 | 312.1865 | 121.076 | 7.44 |
| | Azaperone | C₁₉H₂₂FN₃O | M + H | 328.18197 | 165.07069 | 121.07603 | 8.04 |
| | Carbamazepine | C₁₅H₁₂N₂O | M + H | 237.10224 | 194.0964 | 220.0757 | 11.15 |
| | Chloropromazine | C₁₇H₁₉ClN₂S | M + H | 319.10302 | 86.09643 | 246.01379 | 11.25 |
| | Diazepam | C₁₆H₁₃ClN₂O | M + H | 285.07892 | 154.0415 | 193.0883 | 13.58 |
| | Diphenhydramine | C₁₇H₂₁NO | M + H | 256.16959 | 167.0855 | 152.0621 | 10.03 |
| | hydrochloride | | | | | | |
| | Droperidol | C₂₂H₂₂FN₃O₂ | M + H | 380.17688 | 165.07072 | 194.09726 | 9.35 |
| | Estazolam | C₁₆H₁₁ClN₄ | M + H | 295.0745 | 267.05524 | 205.0755 | 11.44 |
| Macrolides | Clarithromycin | C₃₈H₆₉NO₁₃ | M + H | 748.48417 | 158.11756 | 116.07061 | 11.04 |
| | Tiamulin | C₂₈H₄₇NO₄S | M + H | 494.32986 | 192.10528 | 119.01613 | 10.8 |
| | Erythromycin | C₃₇H₆₇NO₁₃ | M + H | 734.46852 | 158.11756 | 116.07061 | 9.92 |
| | Kitasamycin | C₄₀H₆₇NO₁₄ | M + H | 786.46343 | 558.32727 | 174.11247 | 11.25 |
| | Tilmicosin | C₄₆H₈₀N₂O₁₃ | M + H | 869.57332 | 174.11247 | 88.07569 | 8.93 |
| | Tylosin | C₄₆H₇₇NO₁₇ | M + H | 916.52643 | 772.44778 | 174.11247 | 10.17 |
| anthelmintics | Mebendazole | C₁₆H₁₃N₃O₃ | M + H | 296.10297 | 264.07675 | 105.03349 | 10.85 |
| | Mebendazole-amine | C₁₄H₁₁N₃O | M + H | 238.09749 | 105.03369 | / | 8.45 |
| | 2-aminofluorobenzimidazole | C₁₄H₁₀FN₃O | M + H | 256.08807 | 123.0241 | 113.0397 | 8.74 |
| | Oxfendazole | C₁₅H₁₃N₃O₃S | M + H | 316.07504 | 284.04882 | 223.0576 | 9.26 |
| | Oxibendazole | C₁₂H₁₅N₃O₃ | M + H | 250.11862 | 218.0924 | 176.04545 | 9.33 |
| | Parbendazole | C₁₃H₁₇N₃O₂ | M + H | 248.13935 | 216.1131 | 160.0505 | 10.65 |
| | 5-hydroxythiabendazole | C₁₀H₇N₃OS | M + H | 218.03826 | 191.02736 | / | 6.44 |
| | Tiabendazole | C₁₀H₇N₃S | M + H | 202.04334 | 175.03245 | 131.06037 | 7.12 |
| | Albendazole | C₁₂H₁₅N₃O₂S | M + H | 266.09577 | 234.06956 | 191.01478 | 10.99 |
| | Albendazole sulfone | C₁₂H₁₅N₃O₄S | M + H | 298.0856 | 266.05939 | 224.01244 | 9.4 |
| | Albendazole sulfoxide | C₁₂H₁₅N₃O₃S | M + H | 282.09069 | 240.04347 | 208.01752 | 8.08 |
| | Amino albendazole | C₁₀H₁₃N₃O₂S | M + H | 240.08012 | 198.03317 | 165.05328 | 6.94 |
| | sulfone | | | | | | |
| | Benzimidazole | C₇H₆N₂ | M + H | 119.06037 | 92.0495 | 65.0386 | 4.8 |
| | Febantel | C₂₀H₂₂N₄O₆S | M + H | 447.13328 | 383.08085 | 280.05391 | 14.78 |
| | Lobendazole | C₁₀H₁₁N₃O₂ | M + H | 206.0924 | 160.0505 | 178.0609 | 7.59 |
| | toltrazuril sulfoxide | C₁₈H₁₄F₃N₃O₅S | M − H | 440.05335 | 371.05813 | / | 8.97 |
| | Clopidol | C₇H₇Cl₂NO | M + H | 191.99775 | 86.9996 | 101.01525 | 6.83 |
| | Dinitolmide | C₈H₇N₃O₅ | M − H | 224.03129 | 181.02438 | 77.03858 | 9.91 |
| | Halofuginone | C₁₆H₁₇BrClN₃O₃ | M + H | 414.02146 | 100.07598 | 120.08076 | 8.89 |
| | Levamisole | C₁₁H₁₂N₂S | M + H | 205.0794 | 178.0685 | / | 6.99 |
| Pesticides | Buprofezin | C₁₆H₂₃N₃OS | M + H | 306.16346 | 201.10561 | 116.05285 | 17.73 |
| | Carboxin | C₁₂H₁₃NO₂S | M + H | 236.07398 | 143.01613 | 93.0573 | 12.61 |
| | 3-hydroxycarbofuran | C₁₂H₁₅NO₄ | M + H | 238.10738 | 163.07536 | 135.08044 | 9.09 |
| | Clothianidin | C₆H₈ClN₅O₂S | M + H | 250.016 | 131.96692 | 113.0168 | 8.96 |
| | Cyromazine | C₆H₁₀N₆ | M + H | 167.10397 | 85.05087 | 125.08217 | 3.63 |
| | Diuron | C₉H₁₀Cl₂N₂O | M + H | 233.02429 | 72.04439 | 159.97153 | 12.65 |
| | Ethopabate | C₁₂H₁₅NO₄ | M + H | 238.10738 | 206.08117 | 164.07061 | 10.29 |
| | Fipronil | C₁₂H₄Cl₂F₆N₄OS | M − H | 434.93143 | 329.95845 | 249.95853 | 15.66 |
| | Fluridone | C₁₉H₁₄F₃NO | M + H | 330.11003 | 310.1038 | 290.09757 | 13.62 |
| | Acephate | C₄H₁₀NO₃PS | M + H | 184.01918 | 142.99264 | 112.99968 | 5.61 |
| | Hexazinone | C₁₂H₂₀N₄O₂ | M + H | 253.1659 | 171.08765 | 71.06037 | 10.59 |
| | Imazalil | C₁₄H₁₄C₁₂N₂O | M + H | 297.0556 | 158.97628 | 255.00864 | 10.77 |
| | Imidacloprid | C₉H₁₀ClN₅O₂ | M + H | 256.05958 | 209.05885 | 175.09793 | 9.3 |
| | Linuron | C₉H₁₀Cl₂N₂O₂ | M + H | 249.01921 | 159.97153 | 182.02414 | 14.09 |
| | Metribuzin | CSH₁₄N₄OS | M + H | 215.09611 | 187.10119 | 131.03859 | 11.73 |
| | Myclobutanil | C₁₅H₁₇ClN₄ | M + H | 289.12145 | 70.03997 | 125.01525 | 14.21 |
| | Acetamiprid | C₁₀H₁₁ClN₄ | M + H | 223.0745 | 126.0105 | 98.9997 | 9.58 |
| | Norflurazon | C₁₂H₉ClF₃N₃O | M + H | 304.0459 | 284.03967 | 160.03686 | 12.81 |
| | Propyzamide | C₁₂H₁₁Cl₂NO | M + H | 256.02905 | 189.9821 | 172.95555 | 14.68 |
| | Atrazine | C₈H₁₄ClN₅ | M + H | 216.10105 | 174.0541 | 96.05562 | 12.63 |
| | Simazine | C₇H₁₂ClN₅ | M + H | 202.0854 | 132.0323 | 104.001 | 11.35 |
| | Desethyl atrazine | C₆H₁₀ClN₅ | M + H | 188.06975 | 146.0228 | 104.001 | 9.37 |
| | Azoxystrobin | C₂₂H₁₇N₃O₅ | M + H | 404.1241 | 344.10397 | 329.07949 | 14.2 |
| | Benoxacor | C₁₁H₁₁Cl₂NO₂ | M + H | 260.02396 | 149.08352 | 120.04439 | 14.31 |
| Other | Scopolamine | C₁₇H₂₁NO₄ | M + H | 304.15433 | 156.10191 | 138.09134 | 7 |
| substances | Valnemulin | C₃₁H₅₂N₂O₅S | M + H | 565.36697 | 263.1417 | 72.08078 | 11.15 |
| | Atropine | C₁₇H₂₃NO₃ | M + H | 290.17507 | 124.11208 | 93.06988 | 7.61 |

(6) analyzing the filtrate by the ultra high performance liquid chromatography-quadrupole/electrostatic field orbitrap high resolution mass spectrometer to obtain original data of the sample;
(7) subjecting the original data of the sample to automatic extraction using Trace Finder software within a mass window of 5 ppm; wherein when a compound meets the following conditions at the same time, it indicates that the compound is a suspected positive compound in the sample:
(i) a signal-noise ratio (S/N) of the compound is greater than 3;
(ii) a difference between the measured retention time of the compound and the retention time of the compound in the mass spectrometry database is equal to or less than 0.2 min or within ±2.5% (not to exceed 0.5 min);
(iii) the measured parent ion and one fragment ion of the compound simultaneously match the information in the mass spectrometry database, and a mass accuracy of the parent ion is less than 5 ppm and a mass accuracy of the fragment ion is less than 10 ppm; and
(8) comparing a fragment ion mass spectrometry of the suspected positive compound in the sample with a fragmentation mass spectrometry of standard for further identification; wherein if the mass accuracy between main fragment ions in the two fragment ion mass spectrometries is less than 10 ppm and the main fragment ions in the two fragment ion mass spectrometries have identical relative abundance, it confirms that the suspected positive compound is present in the sample.

2. The screening and confirmation method of claim 1, wherein in step (3), when the sample is muscle or poultry egg, the extracting solution is a mixed solution of 3.0 mL of Mcllvaine-Na₂EDTA buffered solution and 10 mL of acetonitrile, where a concentration of EDTA in the Mcllvaine-Na₂EDTA buffered solution is 0.1 mol/L; when the sample is cow's milk or goat's milk, the extracting solution is 20 mL of acetonitrile.

3. The screening and confirmation method of claim 1, wherein in step (4), the solid phase extraction column is a HyperSep Retain PEP column.

4. The screening and confirmation method of claim 1, wherein in steps (5) and (6), parameters of the ultra high performance liquid chromatography are set as follows:
chromatographic column: C18 (150 mm×3.0 mm, particle size 1.8 μm); column temperature: 30° C.; injection volume: 10 μL; flow rate: 0.3 mL/min;
mobile phase A: 0.1% aqueous formic acid solution for positive ion mode and 0.03% aqueous ammonia solution for negative ion mode; mobile phase B: a solution of 0.1% formic acid in acetonitrile for positive ion mode and a solution of 0.03% ammonia in acetonitrile for negative ion mode; a gradient elution program in positive ion mode is shown in Table 2, and a gradient elution program in negative ion mode is shown in Table 3.
TABLE 2
Gradient elution program in positive ion mode

| Time (min) | A(%) | B(%) |
|---|---|---|
| 0 | 95 | 5 |
| 1.0 | 95 | 5 |
| 15 | 5 | 95 |
| 17 | 5 | 95 |
| 17.1 | 95 | 5 |
| 22.0 | 95 | 5 |

TABLE 3
Gradient elution program in negative ion mode

| Time (min) | A(%) | B(%) |
|---|---|---|
| 0 | 95 | 5 |
| 8 | 5 | 95 |
| 10 | 5 | 95 |
| 10.1 | 95 | 5 |
| 13 | 95 | 5 |

5. The screening and confirmation method of claim 1, wherein in steps (5) and (6), conditions of the mass spectrometry are:
ion source: electron spray ion source (ESI+ and ESI−);
scan mode: full scan and automatically triggered secondary scan mode;
capillary voltage: 3.2 kv (ESI+) and 2.8 kv (ESI−);
ion transmission capillary temperature: 325° C.;
atomizing gas temperature: 350° C.;
sheath gas pressure: 40 arb;
auxiliary gas pressure: 40 arb;
dwell time of the primary mass spectrometry: 100 ms;
dwell time of the secondary mass spectrometry: 60 ms;
scanning range (m/z): 50-1000; and
collision energy: 20, 40 and 60 eV.

6. The screening and confirmation method of claim 1, wherein in step (3), the shaking is performed at 6,000 rpm for 10 min; and the centrifugation is performed at 4,000-7,000 rpm for 5 min.
